(19) United States
(12) Patent Application Publication
(10) Pub. No.: US 2022/0000854 A1
(43) Pub. Date: Jan. 6, 2022

(54) DOMPERIDONE ANTINEURODEGENERATIVE COMBINATIONS AND USE

(71) Applicant: CHASE THERAPEUTICS CORPORATION, Washington, DC (US)

(72) Inventors: Thomas N. CHASE, Washington, DC (US); Kathleen E CLARENCE-SMITH, Washington, DC (US)

(73) Assignee: CHASE THERAPEUTICS CORPORATION, Washington, DC (US)

(21) Appl. No.: 17/291,497

(22) PCT Filed: Dec. 3, 2019
(86) PCT No.: PCT/US2019/064112

§ 371 Date: May 5, 2021

Related U.S. Application Data
(60) Provisional application No. 62/785,602, filed on Dec. 27, 2018.
(60) Provisional application No. 62/817,162, filed on Mar. 12, 2019.
(60) Provisional application No. 62/817,274, filed on Mar. 12, 2019.
(60) Provisional application No. 62/844,347, filed on May 7, 2019.
(60) Provisional application No. 62/845,521, filed on May 9, 2019.

(51) Int. Cl. A61K 31/454; A61K 31/426; A61K 31/138; A61K 31/423; A61K 9/00; A61P 25/16

(52) U.S. Cl. CPC A61K 31/454; A61K 31/426; A61P 25/16; A61K 31/423; A61K 9/0053; A61K 31/138

(57) ABSTRACT

A pharmaceutical combination comprising domperidone, a 6-propylamino-4,5,6,7-tetrahydro-1,3-benzothiazole-2-amine and at least one of fluoxetine, zonisamide or a statin, for the treatment of protein misfolding neurodegenerative diseases.

### RELATED APPLICATIONS

This application claims the benefit of U.S. Provisional Patent Application Ser. No. 62/785,602, filed Dec. 27, 2018, and U.S. Provisional Patent Application Ser. No. 62/817,162, filed Mar. 12, 2019, and U.S. Provisional Patent Application Ser. No. 62/817,274, filed Mar. 12, 2019, and U.S. Provisional Patent Application Ser. No. 62/844,347, filed May 7, 2019, and U.S. Provisional Patent Application Ser. No. 62/845,521, filed May 9, 2019, the disclosures of which are incorporated herein in their entirety by reference.

### FIELD OF THE INVENTION

The present invention pertains to the field of the treatment of neurodegenerative diseases, and in particular, of the treatment of neurotoxic processes due to protein misfolding in neurodegenerative diseases.

### OBJECT OF THE INVENTION

The present invention concerns a pharmaceutical combination comprising domperidone, 6-propylamino-4,5,6,7-tetrahydro-1,3-benzothiazole-2-amine, and at least one synergistic agent such as fluoxetine, zonisamide or a statin, and its use for the treatment of protein misfolding neurodegenerative diseases (or disorders) in humans. A preferred embodiment of the present invention includes the use of domperidone in combination with a selective serotonin reuptake inhibitor (such as fluoxetine), an anticonvulsant (such as zonisamide) or an HMG-CoA reductase inhibitor (such as a statin), for augmenting the synucleinopathy-modifying potential of pramipexole in humans, thus allowing at least a slowing of disease progression at doses that are both safe and tolerable.

### DEFINTIONS

“CNS”: Central Nervous System.
“IR”: Immediate Release of the active ingredient from a composition.
“ER”: Extended Release of the active ingredient from a composition.
“GI”: Gastro-Intestinal.
“AE(s)”: Adverse Effect(s).
“SNCA gene”: Synuclein-alpha or alpha-synuclein gene.
“α-Syn”: α-synuclein or alpha-synuclein.
“A-β”. Amyloid-β.
“TauP”: Tau protein.
“PMND” (Protein Misfolding Neurodegenerative Disease): a disease (or disorder) caused by misfolding, oligomerization and aggregation of brain proteins.
“AD”: Alzheimer's Disease.
“PD”: Parkinson's Disease.
“LBD”: Lewy Body Dementia.
“DLB”: Dementia with Lewy Body.
“HD”: Huntington's Disease.
“CBD”: Corticobasal degeneration.
“FTD-PD17”: frontotemporal dementia with parkinsonism-linked to chromosome 17.
“FTLD”: Frontotemporal Lobe Dementia.
“PSP”: Progressive Supranuclear Palsy.
“PickD”: Pick's Disease.
“GBA”: Mutations in the glucocerebrosidase gene.
“MSA”: Multiple System Atrophy.
“MT”: Multiple Taupathies.
“ALS”: Amyotrophic Lateral Sclerosis.
“SEP”: Spongiform encephalopathies.
“FAP”: Familial amyloidotic polyneuropathy.
“TDDS”: Transdermal Drug Delivery System.
“mers”: this term, preceded by a number-range, is a neologism indicating the number of protein monomers in an oligomer formed in the oligomerization of said protein, as described in the Sengupta et al. 2016 reference, which is incorporated herein by reference in its entirety.
“Dyslipidemia”: a disorder of lipoprotein metabolism, including lipoprotein overproduction or deficiency, as may be manifested by elevation of the total cholesterol, the low-density lipoprotein (LDL) cholesterol and the triglyceride concentrations, and a decrease in the high-density lipoprotein (HDL) cholesterol concentration in the blood, and other blood disorders the statins are indicated for.
“Effective domperidone dose per unit form”: This expression, as used herein, refers to a dose of domperidone or pharmaceutically acceptable salt or solvate thereof that is equivalent to from 2 mg to 120 mg of domperidone base, per unit form.
“Effective daily dose of domperidone”: This expression, as used herein, refers to a dose of domperidone or pharmaceutically acceptable salt or solvate thereof that is equivalent to from 4 mg to 120 mg of domperidone base daily.
“6-propylamino-4,5,6,7-tetrahydro-1,3-benzothiazole-2-amine”: A chiral chemical compound that is available as racemate, chemically (R,S)-6-propylamino-4,5,6,7-tetrahydro-1,3-benzothiazole-2-amine, as (R)-stereoisomer, chemically (R)-6-propylamino-4,5,6,7-tetrahydro-1,3-benzothiazole-2-amine (“dexpramipexole”, INN), and as (S)-stereoisomer, chemically (S)-6-propylamino-4,5,6,7-tetrahydro-1,3-benzothiazole-2-amine (“pramipexole”, INN). These three chemical entities are basic substances that may be isolated each as an acid addition salt and solvate thereof. Pramipexole dihydrochloride monohydrate is also known with its USAN “pramipexole hydrochloride”. As used herein, “6-propylamino-4,5,6,7-tetrahydro-1,3-benzothiazole-2-amine” is a general term that, unless otherwise specified, designates a member selected from the group consisting of pramipexole, the racemate, and a pramipexole/dexpramipexole mixture.
“(R)/(S)-mixture”: This term designates a dexpramipexole/pramipexole physical mixture used as an active ingredient according to the present invention.
“(S)-enantiomer”: This term, as used herein with reference to 6-propylamino-4,5,6,7-tetrahydro-1,3-benzothiazole-2-amine doses (daily or per unit form) designates the (S)-stereoisomer, included in said doses that, in said 6-propylamino-4,5,6,7-tetrahydro-1,3-benzothiazole-2-amine, are primarily responsible for its dopaminergic action, with such dopaminergic action counteracted by domperidone as described herein. More specifically, S-enantiomer is herein used to designate the S-stereoisomer that is present in the racemate or pharmaceutically acceptable salt thereof, and similarly, to designate the pramipexole or pharmaceutically acceptable salt thereof that is present, as (S)-constituent, in a (R)/(S)-mixture, in order to distinguish it from pramipexole used alone.
The terms “6-propylamino-4,5,6,7-tetrahydro-1,3-benzothiazole-2-amine”, “(R)-6-propylamino-4,5,6,7-tetrahydro-1,3-benzothiazole-2-amine”, “dexpramipexole”, “pramipexole”, “(S)-6-propylamino-4,5,6,7-tetrahydro-1,3-benzothiazole-2-amine”, “(S)-enantiomer”, “racemate” and “(R)/(S)-mixture” include the free bases and pharmaceutically acceptable salts and solvates thereof (unless otherwise specified); and the relative doses (daily or per unit form) are given in equivalents of pramipexole dihydrochloride monohydrate.
“Effective daily dose of pramipexole” or “effective daily dose of (S)-enantiomer”: An effective pediatric or adult daily (S)-6-propylamino-4,5,6,7-tetrahydro-1,3-benzothiazole-2-amine or pharmaceutically acceptable salts and solvates thereof dose equivalent to at least the pramipexole dihydrochloride monohydrate approved daily dose for the treatment of PD.
“Effective pramipexole amount (or dose) per unit form” or “effective amount (or dose) per unit form of (S)-enantiomer”: An amount per unit form of pramipexole or pharmaceutically acceptable salt or solvate thereof that is equivalent to at least a pramipexole dihydrochloride monohydrate amount per unit form approved for the treatment of PD. More specifically said amount per unit form is equivalent to from 0.125 mg to 45 mg of pramipexole dihydrochloride monohydrate. As noted above and as used herein, “pramipexole” and “(S)-enantiomer” refer to the same chemical entity, but the term “(S)-enantiomer” is generally used when citing the racemate or (R)/(S)-mixtures.
“Fluoxetine”: 1-methylamino-3-phenyl-3-[4-(trifluoromethyl)phenoxy]propane. Unless otherwise specified, the term fluoxetine (INN) designates the fluoxetine free base and salts and solvates thereof, in particular its hydrochloride salt.
“Zonisamide”: benzo[d]isoxazol-3-ylmethanesulfonamide. Unless otherwise specified, the term zonisamide (INN) designates zonisamide acidic form and alkaline metal salts thereof, in particular its sodium salt.
“Statin”: a class of chemical compounds with a 3,5-dihydroxyheptane or 3,5-dihydroxyhept-6-ene carboxylic acid structure linked, via its 7-position, to a carbocyclic or heterocyclic structure, in some cases in form of 5-lactone thereof, used as medicaments for treating dyslipidemia.
“Effective statin dose per unit form (or dose per unit form)” and “Effective statin daily dose”: a statin dose per unit form or daily dose of from 0.5 mg to 80 mg. According to the structure of each statin, said dose-range refers to an equivalent of the free acid, to an equivalent of a specific salt, or, in case of a lactone, to the lactone itself.
Unless otherwise specified, the terms designating the active principles “domperidone”, “6-propylamino-4,5,6,7-tetrahydro-1,3-benzothiazole-2-amine”, “pramipexole” and “fluoxetine” include the free base and acid addition salts and solvates thereof.
The terms “comprise,” “comprises,” “comprising” “include,” “includes,” and “including” are interchangeable and not intended to be limiting.
   It is to be further understood that where descriptions of various embodiments use the term “comprising,” those skilled in the art would understand that the present disclosure also contemplates such embodiments alternatively described using the language “consisting essentially of” or “consisting of”.
The transitional phrase “consisting essentially of” encompasses the specified materials or steps and those that do not materially affect the basic and novel characteristic(s) of the present invention. For instance, combinations and/or compositions consisting essentially of domperidone and 6-propylamino-4,5,6,7-tetrahydro-1,3-benzothiazole-2-amine, or combinations and/or compositions consisting essentially of domperidone and 6-propylamino-4,5,6,7-tetrahydro-1,3-benzothiazole-2-amine, with at least one of fluoxetine, zonisamide or a statin. Such combinations or compositions of the present invention provide a synergistic effect by augmenting the synucleinopathy-modifying potential of pramipexole or increasing the therapeutic efficacy of pramipexole to allow for at least a slowing of disease progression at doses that are both safe and tolerable for treatment of protein misfolding neurodegenerative diseases (or disorders) in humans.
The transitional phrase “consisting of” excludes element(s), step(s), or material(s) not specified in the claim. For instance, the present invention provides combinations consisting of domperidone and 6-propylamino-4,5,6,7-tetrahydro-1,3-benzothiazole-2-amine, as well as compositions consisting of domperidone, 6-propylamino-4,5,6,7-tetrahydro-1,3-benzothiazole-2-amine, and at least one pharmaceutically acceptable excipient or carrier. The present invention also provides combinations consisting of domperidone, 6-propylamino-4,5,6,7-tetrahydro-1,3-benzothiazole-2-amine, and at least one of fluoxetine, zonisamide or a statin, as well as compositions consisting of domperidone, 6-propylamino-4,5,6,7-tetrahydro-1,3-benzothiazole-2-amine, with at least one of fluoxetine, zonisamide or a statin, and at least one pharmaceutically acceptable excipient or carrier.

### BACKGROUND OF THE INVENTION

Neurodegenerative disorders cause a progressive injury and death of nerve cells in the central nervous system. They include Parkinson's disease, Alzheimer's disease, Huntington's disease, amyotrophic lateral sclerosis and others. All are incurable, resulting in an inexorable loss of neurologic and psychiatric function. Although the clinical features of these disorders differ, they all share one similar characteristic—pathogenesis involving the prion-like misprocessing of normal brain proteins into aggregated forms that are capable of replication, propagation and neurotoxicity. In PD for example the protein is alpha-synuclein.

As research progresses, many similarities appear that relate these diseases to one another on a cellular and sub-cellular level. Discovering these similarities offers hope for therapeutic advances that could ameliorate many diseases simultaneously. There are many known similarities between different neurodegenerative disorders including atypical protein assemblies as well as induced cell death. Neurodegeneration can be found in many different levels of neuronal circuitry ranging from the molecular to the systemic.

The synthesis and misfolding of oligomeric/aggregated species of such proteins as amyloid-β, tau and alpha-synuclein now appear to be the predominant pathology underlying most, if not all, neurodegenerative disorders, including Alzheimer's and Parkinson's disease. Whilst end stage insoluble products of aggregation have been well characterized in human and animal models of disease, increasing evidence from in vitro and in vivo studies indicates that soluble intermediates of aggregation, i.e. oligomers, especially those in the 4 to 20 mers range, may be the key species that mediate toxicity and underlie seeding and spreading in disease (Choi and Gandhi 2018).

A-β is a 38 to 43 amino acid peptide generated by the sequential proteolytic cleavage of amyloid precursor protein (“APP”) by β- and γ-secretases (Chow et al. 2010). It is thought that the over-production of A-β generated from APP plays a role in AD development. Soluble A-β oligomers, on the other hand, have been shown to produce cognitive deficits in the absence of plaques (Gandy et al. 2010). The larger aggregates are not essential to cognitive impairment (Petersen et al. 2013) nor responsible for neurodegeneration and the smaller soluble oligomers are presumed to be the toxic species of A-β. The toxic soluble oligomers are spherical in shape ranging from about 3 to 10 nm. These spheroidal structures come together forming strings of beads, termed protofibrils, which reportedly also possess toxic effects (Glabe, 2006).

Alpha-synuclein, a protein composed of 140 amino acids encoded by the SNCA (Synuclein-Alpha) gene, is abundantly expressed in the human brain and to a lesser extent in various other organs. In brain, alpha-synuclein (hereafter also referred to as simply “synuclein”) is mainly found in neuronal terminals, especially in the cortex, hippocampus, substantia nigra and cerebellum, where it contributes to the regulation of neurotransmitter release, and passes into the peripheral blood stream (Marques and Outeiro, 2012), in part packaged within exosomal vesicles originating from the CNS (Shi et al. 2014).

Many amyloidogenic proteins, such as tau and amyloid-beta (A-β) species, may also be aberrantly converted from their normal monomeric form into soluble intermediates of aggregation, i.e., oligomeric species, that can become neurotoxic. For A-β, toxic oligomers are now thought to range from 8-24 mers while α-synuclein oligomers are 6-18 mers and tau oligomers are 3-15 mers (Sengupta et al 2016), suggesting a universal mechanism of toxicity for amyloid proteins such as tau (Gerson and Kayed 2013, cited in Sengupta et al. 2016), α-synuclein (Sengupta et al. 2016), and TAR DNA-binding protein 43 (TDP-43) (Choksi et al. 2014, Fang et al. 2014, both cited in Sengupta et al. 2016).

Under normal circumstances, these proteins appears to form a stably folded oligomers that resists aggregation. But, in certain pathological conditions, for unknown reasons, they misfold, oligomerize and aggregate (with the eventual formation of fibrils). Somewhere along this aberrant pathway, toxic misfolded protein species are believed to be formed which may also pass into the peripheral (systemic) circulation.

Aberrant protein misfolding, oligomerization and aggregation are now thought to be the cause of PMNDs, notably PD, LBD, DLB, Parkinsonian disorders associated with glucocerebrosidase (GBA) mutations, MSA, AD, HD, multiple tauopathies, and several other disorders, which are collectively referred to as “synucleinopathies”. Alpha-synuclein is a ubiquitous protein that is especially abundant in the brain and has been postulated to play a central role in the pathogenesis of Parkinson's disease (PD), some cases of Alzheimer's disease, and other neurodegenerative disorders (Kim et al. 2004, Sweeney et al. 2017).

Synucleinopathies are generally defined as a group of neurodegenerative disorders characterized in part by the intracellular accumulation of abnormal synuclein aggregates, some of which are toxic and contribute to the pathogenesis of the aforementioned disorders.

An abnormal ratio of monomeric to oligomeric synuclein species, or more specifically an abnormal pattern of changes in the relative amounts of monomeric and oligomeric species of alpha-synuclein contained within brain-derived exosomes in peripheral blood of a patient, is postulated to be a diagnostic hallmark of a synucleinopathy and thus, for example, of one of the aforementioned neurodegenerative disorders of the human CNS.

PD is a common neurodegenerative disorder of the human CNS (Poewe et al. 2017) typically presenting with three major clinical signs: resting tremor, bradykinesia, and muscular rigidity. In addition, postural instability and various neurobehavioral disabilities may occur. In the US alone it is now estimated that well over one million individuals are afflicted by this inexorably progressive disorder. Along with the aging of the American population, prevalence rates and societal costs are expected to rise exponentially. Parkinsonian signs largely reflect a loss of dopamine-containing neurons in the basal ganglia. Drugs now used to relieve symptoms generally act by restoring brain dopaminergic function (Connolly and Lang. 2014). None are known to alter the basic parkinsonian disease process. Indeed, notwithstanding prodigious investigative efforts over the past half-century, the cause and cure of these fatal disorders remain elusive.

LBD is one of the most common types of progressive dementia. The central features of LBD include progressive cognitive decline, visual hallucinations, and parkinsonian motor symptoms, such as slowness of movement, difficulty walking, and muscular rigidity. Some may also suffer from depression. The symptoms of LBD are caused by the selective loss of nerve cells, presumably a result of synuclein misprocessing and associated with the build-up of Lewy bodies, spherical synuclein accumulations inside many of the degenerating neurons. Researchers do not know precisely why alpha-synuclein accumulates into Lewy bodies or how synuclein species can cause the symptoms of LBD. The formation of Lewy bodies has been considered to be a marker for PD; however, Lewy bodies have also been observed in approximately 60% of both sporadic and familial cases of Alzheimer's disease (AD) (Al-Mansoori et al. 2013). Accordingly, the aggregation of α-synuclein has been strongly implicated as a critical step in the development of neurodegenerative diseases (Al-Mansoori et al. 2013).

Sporadic PD or brainstem-predominant type LBD, and dementia with Lewy bodies (DLB) are the two most frequent α-synucleinopathies, and are progressive multisystem neurodegenerative disorders with widespread occurrence of α-synuclein deposits in the central, peripheral, and autonomic nervous system (Jellinger 2008a). Reportedly, there is considerable clinical and pathologic overlap between PD (with or without dementia) and DLB, corresponding to Braak LB stages 5 and 6 (Braak et al. 2003), both frequently associated with variable Alzheimer-type pathology (Jellinger 2008a). Dementia often does not correlate with progressed stages of LB pathology, but may also be related to concomitant Alzheimer lesions or mixed pathologies (Jellinger 2008a).

Alzheimer disease (AD) is characterized by deposition of β-amyloid peptides, phosphorylated tau protein (3- and 4-repeat tau) and frequent α-synuclein (aSyn, as abbreviated by the authors) deposits (Jellinger 2008b). Lewy body diseases, such as sporadic Parkinson disease (PD) and dementia with Lewy bodies (DLB), show a-Synuclein-positive deposits in neurons, neurites, glia, and presynaptic terminals, while frontotemporal dementias present tau-positive and tau-negative, ubiquitin- and TDP-43-positive neuronal and glial inclusions (Jellinger 2008b). Molecular interactions between major proteins, which may occur within the same brain in various distribution patterns, cause variable phenotypes and mixed pathologies, e.g. AD with α-Synuclein pathology in the brainstem and amygdala, PD and DLB with AD lesions, and frontotemporal dementia with a mixture of various deposits, while others are featured by one principal pathology without other lesions (e.g. tangle-predominant type of dementia, pure PD, brainstem-predominant LBD) (Jellinger 2008b). In Alzheimer's disease, amyloid-β and tau proteins become oligomerized and accumulate in brain tissue where they appear to cause neuronal injury and loss; indeed, some aver that such soluble intermediates of aggregation, or oligomers, are the key species that mediate toxicity and underlie seeding and spreading in disease (Cline et al. 2018, Choi and Gandhi 2018).

The aforementioned Transactive Response DNA-binding protein 43 (TDP-43) is a 43 kDa protein encoded in humans by the TAR DBP gene. In Frontotemporal lobe dementia, a mutation of the TAR DBP gene forms highly phosphorylated toxic amyloid TDP-43 oligomers that accumulate in the frontal brain regions of patients suffering from this disease (Fang at al. 2014).

In ALS, fibrils and oligomers have also been regarded as the aggregated protein agents of neuronal dysfunction. More specifically, a corkscrew-like structure in oligomerized superoxide dismutase 1 (SOD1) reportedly can serve as the cytotoxic segment in certain individuals with ALS. Mutations that prevent formation of this structure eliminate cytotoxicity of the segment in isolation as well as cytotoxicity of the ALS-linked mutants of SOD1 in primary motor neurons and in a Danio rerio (zebrafish) model of ALS (Sangwan et al. 2017).

α-Synuclein and tau aggregates can co-exist in several neurodegenerative disorders (PMND), including Parkinson's disease, Alzheimer's disease, and progressive supranuclear palsy (PSP) and indeed there is evidence that α-synuclein enhances the harmful effects of tau, thus contributing to disease progression (Castillo-Carranza et al. 2018; Erro Aguirre et al. 2015). Tau oligomers in biological fluids, in particular in CSF, can be measured by ELISA and Western blot analysis using anti-tau oligomer antibodies (Sengupta et al. 2017).

MSA with orthostatic hypotension is the current term for a neurological disorder that was once called Shy-Drager syndrome. A progressive disorder of the central and autonomic nervous systems, it is characterized by orthostatic hypotension (an excessive drop in blood pressure when standing up), which causes dizziness and fainting. Multiple system atrophy can occur without orthostatic hypotension, but instead have urinary tract involvement (urgency/incontinence). Neurologists classify the disorder into 3 types: the Parkinsonian-type includes symptoms of Parkinson's disease such as slow movement, stiff muscles, and tremor; the cerebellar-type, which causes problems with coordination and speech; and the combined-type, which includes symptoms of both parkinsonism and cerebellar dysfunction. Problems with urinary incontinence, constipation, and sexual impotence may happen early in the course of the disease. Other symptoms include generalized weakness, double vision or other vision disturbances, difficulty breathing and swallowing, sleep disturbances, and decreased sweating. Because the disease resembles others, a correct diagnosis may take years.

Mutations in the glucocerebrosidase gene (GBA) can result in the autosomal recessive disorder Gaucher disease, Different lines of evidence suggest that mutant GBA can be a risk factor for some cases of Parkinson's disease. Indeed, GBA mutations are now thought to be the single largest risk factor for development of idiopathic PD. Clinically, on imaging and pharmacologically, GBA PD is nearly identical to idiopathic PD (O'Regan et al. 2017). The molecular mechanisms which lead to this increased PD risk in GBA mutation carriers are not fully elucidated, but have been shown to be associated with the accumulation of synuclein (Soria et al. 2017).

Corticobasal Degeneration (“CBD”), pathologically classified as tauopathy, presents with various phenotypes some of which include parkinsonian features, especially rigidity and akinesia (Reich and Grill 2009). Only in a few do these manifestations benefit from standard dose dopaminergic therapy, usually only to a moderate degree and with short-lived duration, notwithstanding evidence of neuronal loss in the substantia nigra and a reduction in presynaptic dopamine transporter binding in the striatum.

In Huntington's disease (HD), cleavage of the full-length mutant huntingtin (mhtt) protein into smaller, soluble aggregation-prone mhtt fragments appears to be a key process in the neuropathophysiology of this disorder. Indeed, aggregation and cytotoxicity of mutant proteins containing an expanded number of polyglutamine (polyQ) repeats is a hallmark of several diseases, in addition to HD. Within cells, mutant Huntingtin (mHtt) and other polyglutamine expansion mutant proteins exist as monomers, soluble oligomers, and insoluble inclusion bodies (Mitchell Sontag et al. 2012).

Several other PMNDs have also, albeit less frequently, been considered synucleinopathies. These include Hallevorden-Spatz syndrome, neuronal axonal dystrophy, and some cases of traumatic brain injury. In the case of Hallevorden-Spatz, symptoms include parkinsonism, dystonia, dysphagia/dysarthria, rigidity or stiffness of the limbs, dementia and spasticity.

Many now believe that processes leading to protein oligomerization and aggregation may be central to the cellular injury and destruction occurring in these disorders.

The mechanism of alpha-synuclein aggregation in these PMNDs remains poorly understood. Current evidence suggests the conversion of a soluble alpha helical structure into a beta pleated conformation with subsequent oligomer formation leads to the aggregation, fibrillization and ultimately deposition of synuclein. Certain of the oligomeric forms of synuclein appear highly neurotoxic and could contribute to the neurodegenerative process characterizing PD and related disorders. These characteristics are similar to the aberrant processing of prion proteins that also can become highly neurotoxic. In addition, phosphorylation of alpha-synuclein at the serine-129 residue has been implicated as a contributory factor (Chen et al. 2016). According to Prusiner et al. 2015, a prion form of alpha-synuclein could be a causal agent, especially for multiple system atrophy. Prions are small proteins that also can misfold, oligomerize, aggregate and propagate to other cells. The result in brain is a profound and spreading neurodegenerative process.

Accordingly, inhibiting the initial misfolding, oligomerization and aggregation of certain brain proteins may be beneficial in slowing or even arresting the progression of PMNDs.

Current evidence further suggests that this aberrant misfolding, oligomerization, fribrillization process also involves other brain proteins such as beta-amyloid, tau, and Huntingtin proteins and that certain of these abnormal species thus formed may play a role in the pathogenesis of disorders such as AD, various tauopathies including PSP and HD (Choi and Ghandi, 2018; Nilson et al, 2017; Hoffner and Djian, 2014). Accordingly, drugs that block the formation and/or neurotoxic action of these aberrant species may confer therapeutic benefit to patients suffering from these disorders (Choi and Ghandi 2018; Nilson et al. 2017; Sengupta et al, 2017).

As mentioned above, alpha-synuclein, as well as others of the aforementioned oligomerized species, readily pass into extracellular spaces and have been identified in cerebrospinal fluid, blood, urine, and saliva (Marques and Outeiro 2012). The mechanisms of alpha-synuclein excretion are not fully understood, but studies have demonstrated that at least a fraction of alpha-synuclein is excreted within exosomes, the 40 nm to 100 nm vesicles of endocytic origin (reviewed in Shi et al. 2014). The ratio of monomeric to oligomeric species within exosomes in peripheral blood originating from the CNS could thus reflect disease transient intensity and/or cumulative severity (Shi et al. 2014), thus suggesting that peripheral blood exosomal alpha-synuclein and related species can help monitor the clinical state of a neurodegenerative disease and response to therapy. Similarly, alpha-synuclein levels in brain-derived exosomes have been reported to correlate with severity of impairment in cross-sectional samples from patients with LBD (Stuendl et al. 2016).

Based on the above, drugs that normalize the ratio of monomeric to oligomeric alpha-synuclein species in peripheral blood exosomes deriving from brain should slow or even arrest the neurodegenerative process associated with the synucleinopathies.

Various compositions for the treatment of PD and related disorders that target the aggregation of brain proteins such as synuclein pathway have been proposed. The discovery process primarily involves cellular and animal models of prion- and synuclein-induced neurodegeneration (Prusiner et al. 2015; Visanji et al. 2016). Unfortunately, none of these models has been validated and all are currently regarded uncertain predictors of effects in humans. Nevertheless, these models continue to be widely used in the absence of better discovery techniques.

Pharmaceutical agents currently proposed for consideration in the present invention include, for instance, such small molecules as (S)-6-propylamino-4,5,6,7-tetrahydro-1,3-benzothiazole-2-amine (pramipexole) and fluoxetine, (S)-6-propylamino-4,5,6,7-tetrahydro-1,3-benzothiazole-2-amine (pramipexole) and zonisamide, or (S)-6-propylamino-4,5,6,7-tetrahydro-1,3-benzothiazole-2-amine (pramipexole) and a statin.

Pramipexole and its analogs, alone or in combination with various drugs have also been considered for the treatment of PD and related disorders.

Pramipexole is a synthetic aminothiazole derivative described in U.S. Pat. No. 4,886,812, the contents of which are incorporated herein in their entirety by reference. It is a dopamine agonist of the non-ergoline class (Schneider and Mierau 1987) that has been approved since the late 1990s for the symptomatic treatment of Parkinson's disease (PD) in doses ranging from 0.375 mg/day to 4.5 mg/day, given in 3 equally divided doses (Mirapex® Prescribing Information, July 2016). Pramipexole is supplied in tablets for immediate release containing 0.125 mg, 0.25 mg, 0.5 mg, 1 mg and 1.5 mg of pramipexole dihydrochloride monohydrate; and in tablets for extended release containing 4.5 mg of pramipexole dihydrochloride monohydrate.

Although pramipexole is widely used for the relief of Parkinsonian symptoms, its potential as a disease modifying agent has made it the object of considerable investigative attention.

Pramipexole reportedly diminishes synuclein oligomer formation in vitro (Ono et al. 2013). Related studies suggest that pramipexole inhibits the toxic effects of rotenone on dopaminergic neurons in a mouse PD model while reducing immunoreactivity for alpha-synuclein; additionally, pramipexole decreases the in vitro oligomerization of human wild-type alpha-synuclein by H₂O₂ plus cytochrome c (Inden et al. 2009). Pramipexole has also been observed to inhibit the aggregation of alpha-synuclein in human neuroblastoma SH-SY5Y cells (Kakimura et al. 2001). Importantly, the relative expression of α-synuclein in peripheral blood exosomes has been reported to decline during pramipexole treatment of PD-type patients, especially those manifesting acute symptomatic benefit (Luo et al. 2016); an observation of considerable interest since animal model studies have indicated that changes in peripheral blood exosomal synuclein species correlate with changes in the CNS (Shi et al. 2014).

In addition, it began to be reported that pramipexole can exert neuroprotective effects in various in vitro cellular and in vivo animal models of PD. Mechanisms by which these protective effects may occur remain uncertain. Unfortunately, the protective effects of pramipexole in animal models are generally small and require higher doses than are considered safe and tolerable for human administration. It is thus hardly surprising that pramipexole, in doses approved for the treatment of motor symptoms of PD failed to evidence neuroprotective (i.e., disease modifying) activity in a randomized, controlled, clinical trial involving 535 PD patients (Schapira et al. 2013).

(R)/(S)-mixtures, consisting of pharmaceutical compositions comprising a therapeutically effective amount of dexpramipexole or pharmaceutically acceptable salts and solvates thereof and a therapeutically effective amount of pramipexole or pharmaceutically acceptable salts and solvates thereof, useful for the treatment of PD, are disclosed in US 2008/0014259, the contents of which are incorporated herein in their entirety by reference.

According to US 2008/0014259, both enantiomers are able to confer neuroprotective effects by their ability to accumulate in brain cells, the spinal cord and mitochondria where they exert a positive effect on neurological function that is independent of the dopamine agonist activity of pramipexole. In particular, said document proposes said composition as a neuroprotective agent and a therapeutically effective amount of from about 0.0625 mg to about 6 mg of pramipexole in combination with up to 5000 mg of dexpramipexole. However, this document emphasizes the pramipexole adverse effects due to its dopaminergic action and tends to privilege pramipexole low doses, as also confirmed by the same applicant in the almost concurrent WO 2008/113003 document, the contents of which are incorporated herein in their entirety by reference.

According to US 2013/0116292, the contents of which are incorporated herein in their entirety by reference, dexpramipexole, or pharmaceutically acceptable salts and solvates thereof, acts by slowing the progression of neuronal degeneration and/or by preventing neuronal cell death. However, no further evidence of this possible noteworthy action of dexpramipexole appeared in the literature.

A synthesis of dexpramipexole and of pharmaceutically acceptable salts thereof, in particular dexpramipexole dihydrochloride monohydrate, is described in US 2012/0253047, the contents of which are incorporated herein in their entirety by reference.

Unfortunately, limitations associated with the administration of pramipexole to for example parkinsonian patients limit its use at the potentially higher neuroprotective doses predicted by some animal models. First, mechanisms to explain its putatively beneficial effects on synuclein-related neurotoxicity continue to elude full understanding. Second, effect sizes in animal model studies tend to be small and occur only at relatively high drug doses. Both situations were also observed in the above mentioned report of pramipexole-induced changes in exosomal synuclein in PD patients, which were associated with the administration of the highest—4.5 mg/day—recommended/approved dose of pramipexole (Mirapex® Package Insert; Revised July 2016).

In the aforementioned report of the Luo et al. 2016 reference, although treatment of Parkinson patients with pramipexole at approved therapeutic doses significantly lowered the relative expression of serum alpha-synuclein (compared with pre-treatment values) in peripheral blood, the magnitude of the effect was small. Higher doses of pramipexole could have been more efficacious, but side effects such as vomiting and severe nausea preclude the use of higher doses. For example, in Corrigan et al. 2000, the authors report that doses of 5 mg/day of pramipexole, hardly higher than the maximum recommended dose of 4.5 mg/day (Pramipexole FDA-approved package Insert) caused nausea in 76% of patients and vomiting in 39% of patients. Furthermore, 36% of patients were not able to complete the study, presumably because of intolerable GI adverse events.

In addition, there is no clinical demonstration of a neuroprotective effect of pramipexole, or of any disease modifying action by pramipexole, at the recommended doses, in patients suffering from a PMND such as PD.

The US 2003/0032661 patent document, the content of which is incorporated herein in its entirety by reference, discloses the use of pramipexole therapeutically effective doses for the prevention and/or treatment of generalized seizures (absences, also atypical absences, myoclonic, clonic, tonic, and tonic-clonic seizures), focal (simple and complex focal) and secondary generalized seizures; and affirms that pramipexole can be used in therapeutically effective doses as an anticonvulsant for treating said cerebral seizures. According to this document, pramipexole may be used in doses of about 0.05 mg to 7.5 mg, preferably 0.1 mg to 5 mg per day, at maximum doses of about 5 mg to 7.5 mg of pramipexole per day. In addition, this document affirms that pramipexole may be used to treat the abovementioned conditions in conjunction, for example, with one or more, preferably one of the following substances: carbamazepine, oxcarbamazepine, valproic acid, diphenylhydantoin, ethosuximide, mesuximide, phenobarbital, primidone, benzodiazepines (preferably diazepam, clonazepam or clobazam), corticotrophin, corticoids, bromides (such as potassium bromide), sultiam, acetazolamide, felbamate, gabapentin, lamotrigine, topiramate, vigabatrin, levetiracetam, and zonisamide. This document, however, does not give any information about how said pramipexole and, a fortiori, any combination thereof with other drugs would have been used for treating said seizures, and limits the description of the pramipexole unit forms to a maximum 1 mg strength.

In a case report (Kataoka and Ueno 2014), pramipexole (4.5 mg/day), in combination with levodopa (250-500 mg/day), entcapone (200 mg/day), selegiline (5 mg/day) and zonisamide (25-50 mg/day), was reported to induce hallucinations in one moderate Parkinsonian patient, after immediate-release pramipexole was switched to extended-release pramipexole.

### Fluoxetine

Fluoxetine, 1-methylamino-3-phenyl-3-[4-(trifluoromethyl)phenoxy]propane, is a selective serotonin reuptake inhibitor (SSRI) antidepressant, available in preparations comprising fluoxetine hydrochloride, in an amount per IR-unit form equivalent to 10 mg, 20 mg or 40 mg of fluoxetine base, to be administered once or twice per day (typically as Prozac®). Fluoxetine hydrochloride is also available in a specific preparation (Prozac® Weekly™), in capsules comprising fluoxetine hydrochloride, in an amount per ER-unit form equivalent to 90 mg of fluoxetine base, to be administered once a week (herein below referred to as “90 mg ER-weekly preparation”). Fluoxetine is currently used in the treatment of major depressive disorder, obsessive-compulsive disorder (OCD), bulimia nervosa, panic disorder, and premenstrual dysphoric disorder. When taken by mouth at recommended maintenance IR-doses (20 mg to 80 mg daily in 1 to 2 divided doses), or in the specific 90 mg ER-weekly preparation by patients with these disorders, fluoxetine typically evinces a high degree of efficacy.

The mechanism by which fluoxetine benefits patients with psycho-affective disorders is generally considered to be linked to the drug's ability to augment CNS serotonin-mediated transmission. In addition, however, large fluoxetine doses in rodents have been shown to induce a significant increase in extracellular concentrations of norepinephrine and dopamine after acute systemic administration (Bymaster et al. 2002).

Fluoxetine augments levels of neurotrophic factors such as glial-derived neurotrophic factor (GDNF) and brain-derived neurotrophic factor (BDNF) and, in addition, the effects of fluoxetine in in vivo transgenic models of alpha-synucleinopathy have received careful investigative attention.

It has also been extensively reported to exhibit neuroprotective activity in various cellular and animal models of neurodegenerative disease (Ubhi et al. 2012).

For example, a laboratory study examined the effect of fluoxetine in the MBP1-hα-syntg mice, a model of MSA (Shults et al. 2005).

Fluoxetine can protect against 6-OHDA (6-hydroxydopamine) (Suzuki et al. 2010) and MPTP (1-methyl-4-phenyl-1,2,3,6-tetrahydropyridine) (Chung et al. 2011) induced damage in toxin-induced models of PD.

Fluoxetine was also disclosed to significantly delay amyloid-β-induced paralysis in the Caenorhabditis elegans model of amyloid-β toxicity by reducing amyloid-β oligomers and to increase thermal stress resistance and extend life span, thus suggesting that fluoxetine may have benefit for the treatment of AD by the reduction of proteotoxicity (Keowkase et al. 2010).

Interestingly, fluoxetine has been reported to enhance the activity of pramipexole in a rodent forced swimming test model of depression by a mechanism yet to be precisely elucidated (Rogóz and Skuza 2006).

The document U.S. Pat. No. 6,667,329 (see also WO00/06162), the contents of which are incorporated herewith in their entirety by reference, discloses a combination of pramipexole with another antidepressant for the treatment of depression. Said other antidepressant may be alprazolam, fluoxetine, opipramol, amitriptyline, fluvoxamine, paroxetine, amitriptyline oxide, imipramine, sertraline, chlordiazepoxide, lofepramine, sulpiride, citalopram, maprotiline, tranylcypromine, clomipramine, mianserin, trazodone, quinpirole, mirtazapine, trimipramine, dibenzepin, moclobemide, tryptophan, doxepin, nefazodone, venlafaxine, nortriptyline or viloxazine. According to U.S. Pat. No. 6,667,329, pramipexole combined with another antidepressant has a significantly greater antidepressant activity than either of the two individual components taken alone, the improvement in the effect of pramipexole by the simultaneous administration of another antidepressant having been discovered in tests on rats using the forced swimming test. Said combination may be a fixed-dose combination.

Nevertheless, no evidence has been reported showing that fluoxetine exerts disease modifying effects in humans with a neurodegenerative disease such as PD, or even positively influences the misfolding of neuronal proteins causing a PMND.

In conclusion, notwithstanding the extensive studies on the effects of fluoxetine and pramipexole, separately, on alpha-synuclein processing over the past ten years, the massive existing literature, and the disclosures of US 2008/0014259 and U.S. Pat. No. 6,667,329, no-one has succeeded in safely increasing pramipexole efficacy, because pramipexole currently provides only marginal activity in the treatment of or prevention of progression of Parkinson's disease or related disorders.

The problem of providing effective treatment to patients suffering from a PMND remains unresolved. In fact, during the last seventeen years no-one has disclosed or suggested a combination of domperidone with both pramipexole and fluoxetine, which could be used to provide an effective means for treating PMNDs.

### Zonisamide

Zonisamide (1,2-benzisoxazole-3-methanesulfonamide) is a sulfonamide anticonvulsant approved for use in the adjunctive therapy of adults with partial-onset seizures (“antiseizure indication”); including infantile spasm, mixed seizure types of Lennox-Gastaut syndrome, myoclonic, and generalized tonic clonic seizure.

This drug is commercially available (Zonegran®) and is supplied for oral administration as capsules containing 25 mg or 100 mg zonisamide. In the treatment of epilepsy, oral zonisamide is generally used in daily doses of 200 mg to 600 mg per day, divided in 2 daily doses, and adjusted to maintain serum levels of 15 to 40 μg/ml.

The drug, unrelated to other anticonvulsants, is believed to act, at least in part, by blocking voltage dependent sodium and T-type calcium channels. It is also a weak carbonic anhydrase inhibitor and a modulator of brain GABAergic and glutamatergic neurotransmission.

Zonisamide has also been reported to exhibit protective activity in various neurotoxin-based cellular and animal models of PD.

U.S. Pat. No. 6,342,515 (Masuda and Ochi, see also WO 99/33465), the contents of which are incorporated herein in their entirety by reference, claims the use of zonisamide for treating neurodegenerative diseases such as primary or secondary Parkinson's disease, Huntington's disease, choreic syndrome and dystonic syndrome in mammals (including human). This claim is supported by pharmacological experiments carried out in mice intraperitoneally treated with 1-methyl-4-phenyl-1,2,3,6-tetrahydropyridine (MPTP) 30 mg/kg, once a day for 8 days repetitively.

In MPTP-treated mice, zonisamide administration (20 mg/kg) also reduced the loss of nigral TH-positive neurons while attenuating the associated striatal dopamine depletion (Yokoyama H et al. 2010). In mice that received 6-hydroxydopamine (6-OHDA) to induce hemiparkinsonism, the injection of zonisamide (30 mg/kg) prevented the loss of nigral dopamine neurons.

In an in vitro neurotoxic model obtained by acutely exposing rat corticostriatal slices to rotenone, a selective inhibitor of mitochondrial complex, low concentrations of zonisamide (0.3, 1, 3 and 10 μM) significantly reduced the rotenone-induced toxicity protecting striatal slices from the irreversible loss of corticostriatal field potential amplitude via a GABA-mediated mechanism (Costa et al. 2010).

In more direct relation to neurodegeneration due to the misfolding of proteins such as α-Synuclein, zonisamide inhibits the in vitro oligomerization and aggregation of alpha-synuclein, a key event in the pathogenesis of PD and related disorders (Ono et al. 2013) and exerts protective effects against A53T α-synuclein-induced neurodegeneration in a manner that may be independent of synuclein aggregation in an in vivo rat model (Arawaka et al. 2014). In the latter study, orally administered zonisamide (40 mg/kg/day) significantly delayed the pace of degeneration four weeks after injection of the viral vector for the A53T α-synuclein gene as compared with the control group. This effect lasted at least eight weeks after transgene injection, but appeared to have no impact on the survival of nigrostriatal dopamine neurons. The chronic administration of zonisamide to Engrailed mutant mice, another genetic model of PD, improved the survival of nigrostriatal dopaminergic neurons as well as their striatal dopaminergic terminals and motor function as compared with saline treatment (Sano et al 2015). The mechanism of these protective effects remains uncertain, although brain-derived neurotrophic factor content reportedly increased in the striatum and ventral midbrain of the zonisamide-treated mice compared to saline-treated controls.

Zonisamide, given at relatively low doses (25 mg-50 mg) either alone or with levodopa, has been observed to improve motor symptoms in patients with PD (Grover et al. 2013). However, there are no clinical reports documenting that zonisamide exerts disease modifying effects in humans with a neurodegenerative disease such as PD, or modifies synuclein species in blood exosomes from patients with PD type disorders.

Thus, notwithstanding the disclosures of the aforementioned U.S. Pat. No. 6,342,515 (see also WO 99/33465) and US 2003/0032661 documents, zonisamide has not been used, nor suggested for use, in combination with pramipexole and/or with domperidone for treating PD or any other PMND.

### Statins

Statins constitute a class of widely marketed drugs approved since the late 1980s for the treatment of hyperlipidemia. All act to inhibit the 3-hydroxy-3-methylglutaryl coenzyme A reductase enzyme (HMG-CoA reductase), which plays a critical role in the synthesis of cholesterol. Evidence from large randomized trials shows that statin therapy reduces the risk of major vascular events such as myocardial infarction, strokes, and coronary revascularization procedures (Collins et al. 2016).

For nearly three decades, statins have been regarded as safe and effective in the primary and secondary prevention of cardiovascular disease, especially for reducing the risk of heart attacks, stroke, and certain arterial revascularization procedures.

Studies in cultured human cells as well as in animal models have shown that drugs of this class are copiously yet selectively taken up by the liver, the target organ for cholesterol lowering drugs. Within the liver, it is currently believed that the lipid-modifying effects of statins such as rosuvastatin occur as a result of increasing the number of hepatic LDL receptors on cell-surfaces to enhance the uptake and catabolism of LDL as well as by inhibiting the hepatic synthesis of very low-density lipoproteins (VLDL).

Statins act selectively as competitive inhibitors of HMG-CoA reductase, the rate-limiting enzyme that converts 3-hydroxy3methylglutaryl coenzyme A to mevalonate, a precursor in the synthesis of cholesterol.

Some statins are also reported to have a favorable effect on other disorders including dementia, lung and prostate cancer, and hypertension.

Evidence accumulated over the past two decades also suggests that statins' class drug may show benefits in a wide range of neurologic conditions (Orr 2008) and may be useful in the treatment of CNS diseases (Willey and Elkind 2010). In vitro studies, suggest that drugs of this class, for example simvastatin, could also be used in the treatment of PD and related disorders because they have been reported in animals to protect against the deleterious consequences of dopaminergic neurotoxin MPTP (Roy and Pahan 2011), even though this beneficial action is still controversial (Carrrol J A et al. 2017).

Statins such as atorvastatin (Kumar et al 2012), lovastatin (Lin et al. 2015, Yan et al. 2015), and simvastatin (Kumar et al 2012, Xu et al. 2013) have been reported to protect against the deleterious consequences of the dopaminergic neurotoxin 6-hydroxydopamine (6-OHDA) in brain cell systems. Similarly, in a 6-OHDA-lesioned cell model, simvastatin provides robust neuroprotection against dopaminergic neurodegeneration, possibly in part via antiinflammatory mechanisms and the PI3K/Akt/caspase 3 pathway (Xu et al. 2013).

Statin class drugs also appear to protect against synuclein related neurotoxicity in PD model using rotenone (Kang et al. 2017).

Statin treatment has also been reported to modify the concentration of alpha-synuclein species contained within exosomes collected from the peripheral blood of PD patients (Bar-On et al. 2008).

In a phase 2 clinical trial on 140 patients aged 18-65 years with secondary progressive multiple sclerosis, high-dose simvastatin reduced the annualized rate of whole-brain atrophy compared with placebo, and was well tolerated and safe (Chataway et al 2014).

These results suggested that simvastatin could also possess disease modifying activity in PD and prompted a clinical investigation on 198 patients as part of a clinical trial (PD-STAT), begun in 2015 in the UK as a 24-months Phase-2 that, if successful, would be followed by a Phase-3 clinical trial (Carrol G B and Wyse R K H 2017). To date, no other information about this study seems to be available to the public.

However, practical limitations associated with the safety and tolerability of administering pramipexole to synucleinopathic patients at the high, neuroprotective doses generally predicted by animal models pose a significant challenge. The effects of pramipexole in animal model studies tend to be small and occur only at relatively high doses. As with most pharmaceuticals, higher doses of pramipexole generally produce more frequent and severe adverse effects along with improved therapeutic efficacy. Side effects of the approved doses, often dose-limiting, include nausea, vomiting, somnolence, confusion, postural hypotension, and hallucinations as well as gastrointestinal disturbances (Mirapex Package Insert, Revised July 2016).

In conclusion, the state of the art shows (a) that the efficacy of pramipexole in the treatment of PD is insufficient, (b) the fact that pramipexole possesses a disease modifying ability in patients suffering from a PMND has not been clinically proven; and (c) that, as set forth above, said efficacy is limited by the adverse effects of this drug.

Thus, more than twenty years after the approval of Mirapex®, nineteen years after the disclosure of Masuda-Ochio WO 99/33465, and fifteen years after the publication of US 2003/0032661, as well as other literature, in particular Corrigan et al. 2000, and the above disclosures of US 2008/0014259 and US 2011/0071135, no-one except the present inventors have succeeded in safely increasing pramipexole efficacy.

### SUMMARY OF THE INVENTION

The present invention increases the therapeutic window for pramipexole, thus safely enabling its full neuroprotective efficacy to a degree that delays onset and/or slows symptom progression to a clinically significant extent in patients suffering from a PMND, such as those with PD-like disorders.

According to the present invention, the combined action of domperidone and at least one synergistic agent selected from the group consisting of fluoxetine, zonisamide ord and a statin (herein also referred to as “fluoxetine, zonisamide or a statin”) allows for the safe treatment of a patient suffering from a PMND with pramipexole.

In fact, it has been found that domperidone, by reducing or even abrogating the GI side effects of high doses of pramipexole (nausea, vomiting, acid reflux, and, especially, constipation) enables the MPND-modifying potential of pramipexole. The present inventors have discovered that the effects of (S)-6-propylamino-4,5,6,7-tetrahydro-1,3-benzothiazole-2-amine (pramipexole) on the exosomal biomarker in the peripheral blood of patients suffering from a neurodegenerative disease caused by an aberrant protein misfolding, oligomerization and aggregation in the CNS, such as synucleinopathic disorders caused by an aberrant processing of synuclein, is substantially and unexpectedly improved by the co-administration of domperidone.

It has also been found that the combination of a 6-propylamino-4,5,6,7-tetrahydro-1,3-benzothiazole-2-amine with domperidone acts by normalizing the otherwise abnormal pattern of changes in the relative amounts of monomeric and oligomeric synuclein species in blood exosomes originating from the CNS of patients suffering from a PMND.

It has further been found that, by using domperidone in constant combination with a 6-propylamino-4,5,6,7-tetrahydro-1,3-benzothiazole-2-amine, it is possible to treat a patient suffering from a PMND by maintaining a therapeutically effective 6-propylamino-4,5,6,7-tetrahydro-1,3-benzothiazole-2-amine or pharmaceutically acceptable salt or solvate thereof daily dose with minimal adverse effects.

In addition, it has been found that the effects of (S)-6-propylamino-4,5,6,7-tetrahydro-1,3-benzothiazole-2-amine (pramipexole) on the exosomal biomarker in the peripheral blood of patients with synucleinopathic disorders like PD of the aberrant processing of synuclein is substantially and unexpectedly improved by the co-administration of domperidone.

Furthermore, it has been found that the combination of domperidone with at least one synergistic agent selected from the group consisting of fluoxetine, zonisamide, and statins acts synergistically to at least safely slow the basic degenerative disease process in patients suffering from a PMND and treated with pramipexole.

The present inventors discovered that the effects of (S)-6-propylamino-4,5,6,7-tetrahydro-1,3-benzothiazole-2-amine (pramipexole) on the exosomal biomarker in the peripheral blood of patients with synucleinopathic disorders like PD of the aberrant processing of synuclein were substantially and unexpectedly improved by the co-administration of domperidone and at least one synergistic agent selected from fluoxetine, zonisamide, and a statin. The domperidone/synergistic agent combined action, not only significantly increases the pramipexole clinical effect, but the dose requirement for both pramipexole and fluoxetine, zonisamide, or a statin safely interdicts the basic degenerative disease process in such patients to a clinically meaningful degree.

Furthermore, it has been discovered that a combination with domperidone increases the efficacy of pramipexole and, allows the administration of pramipexole daily doses as high as up to 45 mg, in particular from more than 20 mg to 45 mg, more particularly from 20.25 mg to 45 mg (in pramipexole dihydrochloride monohydrate).

Moreover, it has been found that the combination (including fixed-dose combinations) of domperidone and and at least one of fluoxetine, zonisamide or a statin allows for the safe administration of a pramipexole dose that may be higher, and even much higher, than the pramipexole maximum daily dose recommended for the symptomatic relief of Parkinson's disease. Consequently, an improvement of the conditions of a patient suffering from a PMND is attained.

For example, the combination, including fixed-dose combinations, of domperidone with at least one of fluoxetine, zonisamide or a statin allows for the administration of (S)-6-propylamino-4,5,6,7-tetrahydro-1,3-benzothiazole-2-amine (pramipexole) daily doses that may be equivalent to from 1.1 times to 10 times, from 1.5 to 10 times, from 2.5 to 10 times, from 3 to 10 times, from more than 3.2 times to 10 times, normally from more than 3.2 to 8 times, from more than 3.2 to 6 times, or from more than 3.2 to 5 times higher than the pramipexole dihydrochloride monohydrate maximum recommended dose for the treatment of the symptoms of Parkinson's disease (such as motor symptoms).

The combination of domperidone or of a pharmaceutically acceptable salt or solvate thereof and at least one of fluoxetine, zonisamide, or statin with a 6-propylamino-4,5,6,7-tetrahydro-1,3-benzothiazole-2-amine, in particular pramipexole, or a pharmaceutically acceptable salt or solvate thereof tends to normalize levels of misfolded, oligomerized and aggregate protein, for example synuclein species within the brain-derived exosomes in peripheral blood of patients suffering from a PMND, in particular a synucleinopathy, for example by diminishing the concentration of abnormal synuclein species (congeners) in the patient's exosomal vesicles found therein, to a significant degree at (S)-enantiomer doses that are safe and tolerable thus evidencing that said patients will enjoy neuroprotective benefit.

In particular,
statins potentiate (augment) the ability of pramipexole to alter misfolded protein species in ways indicating the activation of a central neuroprotective mechanism, i.e. reducing oligomerization of said proteins;
these changes occur at safe and tolerable doses of both drugs;
these changes are indicative of CNS changes that will confer disease clinical improvement in a way and to a degree that will provide practical and significant disease modifying benefit to sufferers; and
the addition of domperidone to the statin unexpectedly allows a better response at relatively low pramipexole doses and also the safe administration of high pramipexole doses, with a consequent possibility of safely improving the conditions of patients suffering from a PMND.

The combination of domperidone with at least one of fluoxetine, zonisamide or a statin allows the administration of, for example, pramipexole at doses (per unit form and daily) much higher than the maximum recommended pramipexole doses. Thus, for example, in combination with a domperidone and a statin the pramipexole doses per unit form or daily doses, including pediatric daily doses and doses used in the titration period, may be in the range equivalent to from 0.375 mg to 45 mg of pramipexole dihydrochloride monohydrate. In an adult patient, a pramipexole dihydrochloride monohydrate daily dose may range from more than 4.5 mg to 45 mg, from 6 mg to 45 and from more than 10 mg to 45 mg. Normally, said pramipexole dihydrochloride monohydrate daily dose in an adult patient is from 14.5 mg to 45 mg, from 15 mg to 35 mg, from 15 mg to 30 mg or from 15 mg to 25 mg.

Finally, unlike other antiemetic agents, domperidone acts to accelerate transit, thus being able to counteract the 6-propylamino-4,5,6,7-tetrahydro-1,3-benzothiazole-2-amine (in particular pramipexole) intestinal adverse effects and for example alleviating constipation and bowel pain that, besides nausea and vomiting, are dose-limiting pramipexole adverse effects.

The invention provides a combination (including fixed-dose combinations) of domperidone with at least one synergistic agent selected from the group consisting of fluoxetine, zonisamide and statins, useful for safely increasing the therapeutic doses of 6-propylamino-4,5,6,7-tetrahydro-1,3-benzothiazole-2-amine, in particular of pramipexole, to consequently enable a better neuroprotective response in a patient suffering from a PMND such as PD and related disorders.

According to a preferred embodiment, in said combination, said statin is lovastatin.

The findings of the present invention provide safe treatment for disabling diseases such as PD, LBD, mutations in the glucocerebrosidase (GBA) gene, AD, the Lewy body variant of AD and PD, neurodegeneration with brain iron accumulation, MSA, HD, MT, ALS, SEP, FAP, and other PMNDs.

Domperidone may be used in combination (including a fixed-dose combination) with a dose of 6-propylamino-4,5,6,7-tetrahydro-1,3-benzothiazole-2-amine that is generally currently used for treating neurodegenerative diseases, or with a dose higher than the dose currently used for treating neurodegenerative diseases. The chronic use of this combination, in further combination (including fixed-dose combination) with at least one synergistic agent selected from the group consisting of fluoxetine, zonisamide and statins, improves the symptoms of a PMND, by concurrently mitigating or even eliminating the adverse effects induced by the S-enantiomer that is present in said 6-propylamino-4,5,6,7-tetrahydro-1,3-benzothiazole-2-amine.

In particular, the invention provides
a fixed-dose combination of domperidone and at least one synergistic agent selected from the group consisting of fluoxetine, zonisamide and statins, useful or for use for the treatment of a PMND in combination with 6-propylamino-4,5,6,7-tetrahydro-1,3-benzothiazole-2-amine, in particular pramipexole;
a fixed-dose combination of domperidone and 6-propylamino-4,5,6,7-tetrahydro-1,3-benzothiazole-2-amine, useful or for use for the treatment of a PMND in combination with at least one synergistic agent selected from the group consisting of fluoxetine, zonisamide and statins;
a fixed-dose combination of 6-propylamino-4,5,6,7-tetrahydro-1,3-benzothiazole-2-amine and at least one synergistic agent selected from the group consisting of fluoxetine, zonisamide and statins, useful or for use for the treatment of a PMND in combination with domperidone; and
a fixed-dose combination of domperidone, 6-propylamino-4,5,6,7-tetrahydro-1,3-benzothiazole-2-amine, and at least one synergistic agent selected from the group consisting of fluoxetine, zonisamide and statins useful or for use for the treatment of a PMND.

As stated in the definitions, 6-propylamino-4,5,6,7-tetrahydro-1,3-benzothiazole-2-amine stands for the active principle per se, independently of the salt or solvate of said active principle. The expressions “salt or solvate thereof”, “salts or solvates thereof” and “salts and solvates thereof”, in reference to domperidone or to 6-propylamino-4,5,6,7-tetrahydro-1,3-benzothiazole-2-amine, indicates that the salt of domperidone or of said 6-propylamino-4,5,6,7-tetrahydro-1,3-benzothiazole-2-amine may be solvated with a solvent, normally water.

Thus, the present invention provides a method for treating a patient suffering from a PMND, which comprises administering to a patient in need of said treatment domperidone in combination with 6-propylamino-4,5,6,7-tetrahydro-1,3-benzothiazole-2-amine and with at least one synergistic agent selected from the group consisting of fluoxetine, zonisamide and statins.

The invention also provides
the use of domperidone for the preparation of a medicament for the treatment of a PMND in a patient, in combination with 6-propylamino-4,5,6,7-tetrahydro-1,3-benzothiazole-2-amine, in particular pramipexole or a pharmaceutically acceptable salt thereof, and with at least one synergistic agent selected from the group consisting of fluoxetine, zonisamide and statins; or
domperidone, for use for the treatment of a PMND in a patient, in combination with 6-propylamino-4,5,6,7-tetrahydro-1,3-benzothiazole-2-amine, in particular pramipexole or a pharmaceutically acceptable salt thereof, and with at least one synergistic agent selected from the group consisting of fluoxetine, zonisamide and statins.

Herein below, these two expressions are merged and referred to as “the use of domperidone for the preparation of a medicament (or domperidone for use) for the treatment of a MPND in combination with a 6-propylamino-4,5,6,7-tetrahydro-1,3-benzothiazole-2-amine and at least one of fluoxetine, zonisamide or a statin”.

Pharmaceutically acceptable acid addition salts and solvates of domperidone and of the 6-propylamino-4,5,6,7-tetrahydro-1,3-benzothiazole-2-amine, as well as pharmaceutically acceptable salts, in particular acid addition salts of fluoxetine, alkaline metal (in particular sodium) salts of zonisamide and alkaline and alkaline-earth salts, of the statins are also included in the method (or use) of the present invention.

In particular, the method (or use) for treating a patient suffering from a PMND according to the present invention comprises treating said patient with an effective daily dose of domperidone in combination with an effective daily dose of 6-propylamino-4,5,6,7-tetrahydro-1,3-benzothiazole-2-amine and an effective daily dose of at least one of fluoxetine, zonisamide or a statin. Normally, said effective domperidone daily dose is equivalent to from 4 mg to 120 mg of domperidone base.

Preferably, in said method (or use), domperidone is administered at the above daily dose, in combination with an effective daily dose of 6-propylamino-4,5,6,7-tetrahydro-1,3-benzothiazole-2-amine and with an effective daily dose of at least one of fluoxetine, zonisamide or statin.

In this combination, the above domperidone daily dose may vary according to the dose of 6-propylamino-4,5,6,7-tetrahydro-1,3-benzothiazole-2-amine, in particular pramipexole, administered therewith, as described in “The 6-propylamino-4,5,6,7-tetrahydro-1,3-benzothiazole-2-amine Component (b)” section below, and also, even though to a lesser extent, with the daily dose of the synergistic agent, as described in “The synergistic agent Component (c)” section below. By consequence, in this combination, within the above domperidone daily dose range, said daily dose (including pediatric doses and doses used in the titration period) may be equivalent to to from 4 mg to 120 mg, from 4 mg to 100 mg, from 4 mg to 80 mg, from 4 mg to 60 mg, from 4 mg to 40 mg, from 4 mg to 30 mg or from 4 mg to 20 mg, normally from 10 mg to 120 mg, from 10 mg to 100 mg, from 10 mg to 80 mg, from 10 mg to 60 mg, from 10 mg to 40 mg, from 10 mg to 30 mg or from 10 mg to 20 mg of domperidone base.

According to the invention, said effective daily dose of 6-propylamino-4,5,6,7-tetrahydro-1,3-benzothiazole-2-amine, including pediatric doses and low doses used in the titration period, is equivalent to from 0.375 mg to 3000 mg of pramipexole dihydrochloride monohydrate, including a (S)-enantiomer daily dose equivalent to from 0.375 mg to 45 mg of pramipexole dihydrochloride monohydrate.

For this treatment, according to the above method (or use) the domperidone, the 6-propylamino-4,5,6,7-tetrahydro-1,3-benzothiazole-2-amine and the fluoxetine, or the zonisamide or the statin are each formulated in a pharmaceutical composition in admixture with a pharmaceutical carrier or vehicle and concurrently or sequentially administered to a patient suffering from a PMND.

According to an advantageous alternative, domperidone and at least one of fluoxetine or zonisamide or a statin may be mixed together (fixed-dose combinations), and formulated in a pharmaceutical composition in admixture with a pharmaceutical carrier or vehicle, to be administered to a patient suffering from a PMND, in combination with 6-propylamino-4,5,6,7-tetrahydro-1,3-benzothiazole-2-amine. This composition is concurrently or sequentially administered to a patient suffering from a PMND, in combination with 6-propylamino-4,5,6,7-tetrahydro-1,3-benzothiazole-2-amine also formulated in a pharmaceutical composition in admixture with a pharmaceutical carrier or vehicle. For this concurrent or sequential administration in combination with 6-propylamino-4,5,6,7-tetrahydro-1,3-benzothiazole-2-amine, a particularly advantageous pharmaceutical composition in dosage unit form comprises a pharmaceutical carrier or vehicle and, as active ingredients, domperidone and lovastatin in a fixed-dose combination.

This domperidone/fluoxetine, zonisamide or statin fixed-dose combination has the advantage of allowing a flexibility in the dosage and in the mode of administration of the 6-propylamino-4,5,6,7-tetrahydro-1,3-benzothiazole-2-amine, in particular of pramipexole, in the treatment of a patient suffering from a PMND with the domperidone/6-propylamino-4,5,6,7-tetrahydro-1,3-benzothiazole-2-amine/fluoxetine, zonisamide or statin combination of the present invention.

Furthermore, the domperidone and the 6-propylamino-4,5,6,7-tetrahydro-1,3-benzothiazole-2-amine may also be mixed together (fixed-dose combination) and formulated in a pharmaceutical composition in admixture with a pharmaceutical carrier or vehicle. This composition is destined to be concurrently or sequentially administered to a patient suffering from a PMND, in combination with fluoxetine, zonisamide or a statin, also each formulated in a pharmaceutical composition in admixture with a pharmaceutical carrier or vehicle. This domperidone/6-propylamino-4,5,6,7-tetrahydro-1,3-benzothiazole-2-amine fixed-dose combination may be administered to a patient suffering from a PMND in combination with the specific fluoxetine 90 mg ER-weekly preparation.

Moreover, domperidone may be formulated in a pharmaceutical composition, in admixture with a pharmaceutical carrier or vehicle, to be administered to a patient suffering from a PMND, in combination with a pharmaceutical composition comprising a pharmaceutical carrier or vehicle and a fixed-dose combination of 6-propylamino-4,5,6,7-tetrahydro-1,3-benzothiazole-2-amine and at least one of fluoxetine or zonisamide or statin, this composition being normally formulated in a dosage unit form.

Fixed-dose combinations, comprising pramipexole and fluoxetine that can be used in further combination with domperidone according to the present invention are disclosed in WO 2018/191408. Fixed-dose combinations, comprising pramipexole and zonisamide that can be used in further combination with domperidone according to the present invention, are disclosed in WO 2018/217845. However, according to the present invention, daily doses of 6-propylamino-4,5,6,7-tetrahydro-1,3-benzothiazole-2-amine, in particular pramipexole, that are higher, and even much higher than that disclosed in said documents may be safely administered to a patient suffering from a PMND thanks to the combined action of domperidone and at least one of fluoxetine, zonisamide or a statin, as set forth above.

Finally, the domperidone, the 6-propylamino-4,5,6,7-tetrahydro-1,3-benzothiazole-2-amine, preferably pramipexole, and fluoxetine, zonisamide or a statin are mixed together (fixed-dose combination) and formulated in a pharmaceutical composition to be administered to a patient suffering from a PMND. Consequently, the invention also provides the use of domperidone for the preparation of a medicament (or a domperidone for use) for the treatment of a PMND in a pharmaceutical composition comprising a pharmaceutical carrier or vehicle and a fixed-dose combination of domperidone, a 6-propylamino-4,5,6,7-tetrahydro-1,3-benzothiazole-2-amine, preferably pramipexole, and at least one synergistic agent selected from the group consisting of fluoxetine, zonisamide and statins.

According to an advantageously embodiment, the invention provides
a pharmaceutical composition comprising a pharmaceutically acceptable carrier or vehicle and a fixed dose combination of domperidone, a 6-propylamino-4,5,6,7-tetrahydro-1,3-benzothiazole-2-amine and at least one of fluoxetine, zonisamide or a statin;
a pharmaceutical composition for use in treatment of a PMND in a patient, comprising a pharmaceutically acceptable carrier or vehicle and a fixed dose combination of domperidone, a 6-propylamino-4,5,6,7-tetrahydro-1,3-benzothiazole-2-amine and at least one of fluoxetine, zonisamide or a statin; and
the use of domperidone for the preparation of a medicament (or domperidone for use) for the treatment of a PMND in a patient, in a fixed-dose cobination of said domperidone, a 6-propylamino-4,5,6,7-tetrahydro-1,3-benzothiazole-2-amine and at least one of fluoxetine, zonisamide or a statin.

Preferably, said domperidone is selected from the group consisting of domperidone free base, domperidone hydrochloride, domperidone maleate and domperidone succinate (1:1); said 6-propylamino-4,5,6,7-tetrahydro-1,3-benzothiazole-2-amine is selected from the group consisting of pramipexole and pharmaceutically acceptable salts and solvates thereof; and said at least one synergistic agent is selected from the group consisting of fluoxetine base, fluoxetine hydrochloride, zonisamide free acid and lovastatin.

In said compositions, said domperidone is present in an amount per unit form equivalent to from 2 mg to 120 mg of domperidone base, said 6-propylamino-4,5,6,7-tetrahydro-1,3-benzothiazole-2-amine is present in an amount equivalent to from 0.125 mg to 3000 mg, including a (S)-enantiomer amount per unit form equivalent to form 0.125 mg to 45 mg of pramipexole dihydrochloride monohydrate, and said at least one synergistic agent is selected from the group consisting of
said fluoxetine is present in an amount equivalent to from 2 mg to 90 mg of fluoxetine base,
said zonisamide is present in an amount equivalent to from 25 mg to 600 mg of zonisamide free acid, and
said statin is present in an amount per unit form of from 0.5 mg to 80 mg.

Normally, said compositions are in dosage unit form and said amounts of the active ingredient are per unit form.

Preferably, in said compositions,
said domperidone is selected from the group consisting of domperidone base, domperidone maleate and domperidone succinate (1:1), in an amount per unit form equivalent to from 2 mg to 120 mg of domperidone base;
said 6-propylamino-4,5,6,7-tetrahydro-1,3-benzothiazole-2-amine is pramipexole or a pharmaceutically acceptable salt or solvate thereof, in an amount per unit form equivalent to from 0.125 mg to 45 mg of pramipexole dihydrochloride monohydrate; and
said statin is lovastatin, in an amount per unit form of from 2.5 mg to 80 mg or from 5 mg to 80 mg, normally from 10 mg to 60 mg.

The above amounts per unit form of domperidone, 6-propylamino-4,5,6,7-tetrahydro-1,3-benzothiazole-2-amine (pramipexole), and fluoxetine, zonisamide or statin include low amounts used in pediatric patients or during the titration period.

According to each of the above alternatives, in the method (or use) for the treatment of a PMND,

said domperidone in said pharmaceutical composition is administered to said patient at a daily dose equivalent to from 4 mg to 120 mg of domperidone base;

said 6-propylamino-4,5,6,7-tetrahydro-1,3-benzothiazole-2-amine in said composition is administered to said patient in need of said treatment at a daily dose that is equivalent to from 0.375 mg to 3000 mg, normally from 1.5 mg to 3000 mg of pramipexole dihydrochloride monohydrate, said daily dose including an (S)-enantiomer daily dose equivalent to from 0.375 mg to 45 mg, normally from 1.5 mg to 45 mg of pramipexole dihydrochloride monohydrate; and said statin in said pharmaceutical composition is administered to a patient in need of
said treatment at a daily dose of from 0.5 mg to 80 mg, normally from 2.5 mg to 80 mg.

Said fluoxetine in said compositions may be administered once or twice a day at a daily dose (in fluoxetine base) of from 4 mg to 90 mg or, in the specific 90 mg-weekly preparation, once a week.

Said zonisamide in said compositions may be administered at a daily dose (in zonisamide free acid) of from 25 mg to 600 mg.

Said statin in said compositions may be administered at a daily dose of from 0.5 mg to 80 mg.

In certain embodiments of the method (or use) described above, said 6-propylamino-4,5,6,7-tetrahydro-1,3-benzothiazole-2-amine with said fluoxetine, said zonisamide or said statin may be administered to said patient in a fixed-dose combination wherein said 6-propylamino-4,5,6,7-tetrahydro-1,3-benzothiazole-2-amine and at least one of said fluoxetine or said zonisamide or said statin are mixed together, and with a pharmaceutical carrier or vehicle in a pharmaceutical composition. This fixed-dose combination is then combined with domperidone, in a pharmaceutical composition in admixture with a pharmaceutical carrier or vehicle. Due to the protective action of the domperidone/synergistic agent combination, the above fixed-dose combination may comprise a high amount of 6-propylamino-4,5,6,7-tetrahydro-1,3-benzothiazole-2-amine per unit form, in particular, a high amount of pramipexole per unit form.

Preferably, said 6-propylamino-4,5,6,7-tetrahydro-1,3-benzothiazole-2-amine is pramipexole base or pramipexole dihydrochloride monohydrate and said statin is lovastatin.

Preferably, according to each of the above alternatives, in the method (or use) for treating a patient suffering from a PMND said domperidone is as free base or as a salt or solvate thereof such as domperidone hydrochloride, domperidone maleate or domperidone succinate (1:1), said 6-propylamino-4,5,6,7-tetrahydro-1,3-benzothiazole-2-amine is pramipexole as free base or as a salt or solvate thereof such as pramipexole dihydrochloride monohydrate, and said statin is lovastatin.

In a preferred embodiment of the method (or use) described above, said domperidone and at least one of said fluoxetine, said zonisamide or said statin combination may be administered to said patient in a fixed-dose combination, wherein said domperidone and at least one of said fluoxetine, said zonisamide or said statin are mixed together and with a pharmaceutical carrier or vehicle in a pharmaceutical composition and administered to a patient suffering from a MPND in combination with 6-propylamino-4,5,6,7-tetrahydro-1,3-benzothiazole-2-amine, also in a pharmaceutical composition in admixture with a pharmaceutical carrier or vehicle.

Preferably and more particularly, according to each of the above alternatives, in the method (or use) for the treatment of a PMND,
said domperidone is selected from the group consisting of domperidone base, domperidone hydrochloride, domperidone maleate and domperidone succinate (1:1), administered at a daily dose equivalent to from from 4 mg to 120 mg of domperidone base;
said 6-propylamino-4,5,6,7-tetrahydro-1,3-benzothiazole-2-amine in said composition is pramipexole or a pharmaceutically acceptable salt or solvate thereof, administered at a daily dose, including pediatric doses and doses used in the titration period, equivalent to from 0.375 mg to 45 mg of pramipexole dihydrochloride monohydrate; and
said fluoxetine in said pharmaceutical composition is administered to a patient in need of said treatment at a daily dose that is equivalent to from 4 mg to 90 mg, normally from 4 mg to 80 mg of fluoxetine base, and/or
said zonisamide in said pharmaceutical composition is administered to a patient in need of said treatment at a daily dose that is equivalent to from 25 mg to 600 mg, normally from 200 mg to 600 mg of zonisamide free acid, and/or
said statin in said pharmaceutical composition is administered to a patient in need of said treatment at a daily dose of from 0.5 mg to 80 mg, normally from 2.5 mg to 80 mg.

Said daily dose of said statin may be lower than the maximum daily dose approved for the treatment of dyslipidemia. Preferably, said statin is selected from the group consisting of lovastatin, at a daily dose of from 5 mg to 80 mg, normally from 5 mg to 60 mg or from 10 mg to 60 mg, and rosuvastatin calcium, at a daily dose of from 2.5 mg to 40 mg, normally from 2.5 mg to 30 mg.

The above daily doses of domperidone, 6-propylamino-4,5,6,7-tetrahydro-1,3-benzothiazole-2-amine (pramipexole), and at least one of fluoxetine, or zonisamide, or statin include low daily doses used in pediatric patients or during the titration period.

The present invention further provides a kit or package comprising a pharmaceutical combination or pharmaceutical compositions as described herein, and instructions for use of the same for treatment of a PMND, for example a synucleinopathy, in a patient in need thereof.

### DETAILED DESCRIPTION

The present invention is based on the discovery that the combination of domperidone and at least one synergistic agent selected from the group consisting of fluoxetine, zonisamide, a statin, synergistically and substantially improves the ability to provide safe and tolerable pramipexole doses to reduce the presence of toxic oligomers of neuronal proteins of patients suffering from a PMND, and thus benefit patients with such fatal disorders to a previously unrealized degree. The invention is also based on the discovery that the combination of domperidone with at least one of fluoxetine, or zonisamide or a statin acts by potentiating the neuroprotective effect of pramipexole and also by allowing the safe administration of high, and even very high pramipexole daily doses.

Thus the invention provides a pharmaceutical combination comprising a domperidone Component (a); a 6-propylamino-4,5,6,7-tetrahydro-1,3-benzothiazole-2-amine, in particular pramipexole, Component (b); and at least one synergistic agent selected from the group consisting of fluoxetine, zonisamide, and statins Component (c), for its use in the treatment of a PMND in a patient.

In this combination, the three components may be
each in a pharmaceutical composition in dosage unit form comprising, respectively, said Component (a), (b) and (c), each in admixture with a pharmaceutical carrier or vehicle, herein below referred to as “combination (a/b/c)”; or
a mixture of Component (a), Component (b), and a pharmaceutical carrier or vehicle, in a pharmaceutical composition in dosage unit form, herein below referred to as “Component (ab)”; and Component (c), in a pharmaceutical composition in dosage unit form in admixture with a pharmaceutical carrier or vehicle, the whole being herein below referred to as “combination (ab/c)”; or
a mixture of Component (a), Component (c), and a pharmaceutical carrier or vehicle, in a pharmaceutical composition in dosage unit form, herein below referred to as “Component (ac)”; and Component (b), in a pharmaceutical composition in dosage unit form in admixture with a pharmaceutical carrier or vehicle, the whole being herein below referred to as “combination (ac/b)”; or
a mixture of Component (b), Component (c), and a pharmaceutical carrier or vehicle, in a pharmaceutical composition in dosage unit form, herein below referred to as “Component (bc)”; and Component (a), in a pharmaceutical composition in dosage unit form in admixture with a pharmaceutical carrier or vehicle, the whole being herein below referred to as “combination (bc/a)”; or
a mixture of Component (a), Component (b), Component (c), and a pharmaceutical carrier or vehicle, in a pharmaceutical composition in dosage unit form herein below referred to as fixed-dose “combination (abc)” or “fixed-dose combination (abc)” or “composition (abc)”.

In particular, the invention provides a combination (including fixed-dose combinations) of domperidone with at least one of fluoxetine, zonisamide or statin, for safely increasing pramipexole doses in the treatment of a PMND.

Specifically, the present invention provides
a method for the treatment of a PMND in a patient in need of said treatment, which comprises administering to said patient domperidone in combination with an effective daily dose of a 6-propylamino-4,5,6,7-tetrahydro-1,3-benzothiazole-2-amine, in particular including pramipexole, and with an effective daily dose of at least one synergistic agent selected from the group consisting of fluoxetine, zonisamide and statins; and
the use of domperidone for the preparation of a medicament (or domperidone for use) for the treatment of patients suffering from a PMND, in combination with an effective daily dose of 6-propylamino-4,5,6,7-tetrahydro-1,3-benzothiazole-2-amine, in particular including pramipexole, and with an effective daily dose of at least one synergistic agent selected from the group consisting of fluoxetine, zonisamide and statins.

In the context of said method (or use), the present invention also provides Component (ab) for the treatment of a PMND in combination with Component (c), Component (ac) for the treatment of a PMND in combination with Component (b), and Component (a) for the treatment of a PMND in combination with Component (bc).

In the context of said method (or use), the present invention also provides a pharmaceutical composition (abc) for use for the treatment of a PMND, comprising a pharmaceutical carrier and a fixed-dose combination of a domperidone Component (a), a 6-propylamino-4,5,6,7-tetrahydro-1,3-benzothiazole-2-amine Component (b) and at least one synergistic agent selected from the group consisting of fluoxetine, zonisamide and statins Component (c).

According to the present invention, due to the protective action of domperidone and the synergistic action of both domperidone and at least one of fluoxetine, zonisamide or a statin, said method (or use) provides, at least,
a safe and high increase of the 6-propylamino-4,5,6,7-tetrahydro-1,3-benzothiazole-2-amine (in particular of pramipexole) daily doses;
a better response to the therapy at the lower 6-propylamino-4,5,6,7-tetrahydro-1,3-benzothiazole-2-amine (in particular of pramipexole) daily doses,
   with the consequent ability of arresting or at least slowing the progression of said PMND in said patient.

The further benefit of early discovery of the disease condition derives from ongoing studies directed at the determination of, for example, misfolded exosomal α-synuclein in the human peripheral blood.

### The Domperidone Component (a)

Domperidone, 5-chloro-1-(1-[3-(2-oxo-2,3-dihydro-1H-benzo[d]imidazol-1-yl)propyl]piperidin-4-yl)-1H-benzo[d]imidazol-2(3H)-one

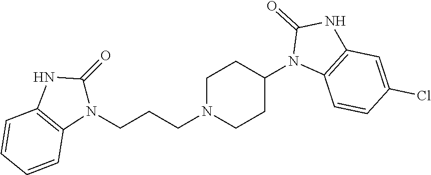 [SMILES: O=c1[nH]c2ccccc2n1CCCN1CCC(n2c(=O)[nH]c3cc(Cl)ccc32)CC1]

is a dopamine-2 receptor antagonist. It acts as an antiemetic and a prokinetic agent through its effects on the chemoreceptor trigger zone and motor function of the stomach and small intestine (Reddymasu et al. 2007), and is reputed not to enter the CNS (Brogden et al. 1982).

A study (Samuels et al. 2007) to investigate the effects of the D₂-receptor agonist pramipexole (at a dose of 0.5 mg/day) with and without the co-administration of the peripherally acting D₂-receptor antagonist domperidone (at a daily dose of 40 mg), on measures of alertness, autonomic and endocrine function, reports that a high enough concentration of the drug crosses the blood-brain barrier to partially antagonize some of the autonomic actions of pramipexole. In particular, this report provides a cautionary note to the use of domperidone alongside pramipexole where the results of interest are those from pramipexole alone.

As set forth above, by combining domperidone with at least one synergistic agent selected from the group consisting of fluoxetine, zonisamide and statins and with pramipexole according to the present invention, not only does the pramipexole effect size become clinically significant by using pramipexole and statin doses falling into the range considered safe and tolerable for human subjects by concurrently and safely interdicting the basic degenerative disease process in patients, but it is also possible to increase the pramipexole dose to a high degree.

The domperidone Component (a) is selected from the group consisting of domperidone base and pharmaceutically acceptable salt and solvates thereof.

Illustrative examples of pharmaceutically acceptable salts of domperidone include acid addition salts with, for example, hydrochloric acid, hydrobromic acid, sulfuric acid, nitric acid, phosphoric acid, acetic acid, propanoic acid, hydroxyacetic acid, 2-hydroxypropanoic acid, 2-oxopropanoic acid, propanedioic acid, butanedioic acid, (Z)-2-butenedioic acid (fumaric acid), (E)-2-butenedioic acid (maleic acid), 2-hydroxybutanedioic acid, 2,3-dihydroxybutanedioic acid, 2-hydroxy-1,2,3-propanetricarboxylic acid, benzoic acid, 3-phenyl-2-propenoic acid, .alpha.-hydroxybenzeneacetic acid, methanesulfonic acid, ethanesulfonic acid, benzenesulfonic acid, 4-methylbenzensulfonic acid, cyclohexanesulfamic acid, 2-hydroxybenzoic acid, 4-amino-2-hydroxybenzoic acid. These salts are disclosed in U.S. Pat. No. 4,066,772, the contents of which are incorporated herewith in their entirety for reference. The solvation solvent is normally water.

A butanedioic acid addition salt, domperidone succinate 1:1, is particularly interesting because it could improve domperidone bioavailability, particularly in the fed state, and may reduce inter-patient variability, thus being advantageous for a pharmaceutical use (Bruni et al. 2013). Also, domperidone hydrochloride and domperidone maleate are particularly advantageous. Domperidone hydrochloride (Latha et al. 2012) and domperidone maleate (Shirisha et al. 2017) have been proposed in TDDS formulations.

In the method, use and combination, including fixed-dose combinations of the present invention, said domperidone Component (a) is present in an amount per unit form equivalent to from 2 mg to 120 mg of domperidone base; and is administered at a daily dose (in domperidone base) of from 4 mg to 120 mg.

For its administration to a patient suffering from a PMND, including pediatric patients, in combination with pramipexole Component (b) and at least one of fluoxetine, zonisamide or statin Component (c), domperidone is formulated in a pharmaceutical composition in dosage unit form comprising, as an active ingredient, said domperidone, in admixture with a pharmaceutical carrier or vehicle.

An advantageous domperidone Components (a) is selected from the group consisting of domperidone base, domperidone hydrochloride, domperidone maleate and domperidone succinate (1:1).

According to the present invention, domperidone is used in a pharmaceutical composition comprising, as an active ingredient, said domperidone in an amount per unit form equivalent to from 2 mg to 120 mg of domperidone base, in admixture with a pharmaceutical carrier or vehicle, and is administered at a daily dose equivalent to from 4 mg to 120 mg of domperidone base, in combination with 6-propylamino-4,5,6,7-tetrahydro-1,3-benzothiazole-2-amine at a daily dose equivalent to from to from 0.375 mg to 3000 mg or from 3 mg to 3000 mg of pramipexole dihydrochloride monohydrate, including a (S)-enantiomer daily dose equivalent to from 0.375 mg to 45 mg or from 3 mg to 45 mg of pramipexole dihydrochloride monohydrate; and with fluoxetine at a daily dose equivalent to from 4 mg to 90 mg, and/or zonisamide at a daily dose of from 25 mg to 600 mg, and/or a statin at a daily dose of from 0.5 mg to 80 mg.

As set forth above, the aforementioned domperidone daily dose may vary according to the daily dose of 6-propylamino-4,5,6,7-tetrahydro-1,3-benzothiazole-2-amine, in particular pramipexole, administered therewith, as described in “The 6-propylamino-4,5,6,7-tetrahydro-1,3-benzothiazole-2-amine Component (b)” section below, and also, even though to a lesser extent, with the daily dose of fluoxetine, zonisamide or statin, as described in “The synergistic agent Component (c)” section below.

In particular, the domperidone Component (a), in the above composition, is administered at a daily dose equivalent to a range selected from the group consisting of from 4 mg to 120 mg, from 4 mg to 100 mg, from 4 mg to 80 mg, from 4 mg to 60 mg, from 4 mg to 40 mg, from 4 mg to 30 mg or from 4 mg to 20 mg, normally from 10 mg to 120 mg, from 10 mg to 100 mg, from 10 mg to 80 mg, from 10 mg to 60 mg, from 10 mg to 40 mg, from 10 mg to 30 mg or from 10 mg to 20 mg of domperidone base. Preferably, said domperidone is selected from the group consisting of domperidone base, domperidone hydrochloride, domperidone maleate and domperidone succinate (1:1).

A composition comprising domperidone as illustrated above, as Component (a), is administered to a patient suffering from a PMND in combination with

6-propylamino-4,5,6,7-tetrahydro-1,3-benzothiazole-2-amine, also in a pharmaceutical composition in dosage unit form, as Component (b), comprising said 6-propylamino-4,5,6,7-tetrahydro-1,3-benzothiazole-2-amine in an amount per unit form equivalent to from 0.125 mg to 3000 mg of pramipexole dihydrochloride monohydrate, inclusive of a (S)-enantiomer amount per unit form equivalent to from 0.125 mg to 45 mg of pramipexole dihydrochloride monohydrate, in particular
as a (R)/(S)-mixture, in an amount per unit form equivalent to from 50 mg to 3000 mg of pramipexole dihydrochloride monohydrate, said amount per unit form including a (S)-enantiomer amount equivalent to from 0.125 to 45 mg of pramipexole dihydrochloride monohydrate;
as a racemate, in an amount equivalent to from 0.25 mg to 90 mg of pramipexole dihydrochloride monohydrate; or
as pramipexole or a pharmaceutically acceptable salt thereof, in an amount equivalent to from 0.125 mg to 45 mg of pramipexole dihydrochloride monohydrate; and with

At least one synergistic agent Component (c) selected from the group consisting of
fluoxetine also in a pharmaceutical composition comprising said fluoxetine in an amount per unit form (in fluoxetine base) of from 2 mg to 90 mg,
zonisamide also in a pharmaceutical composition comprising said zonisamide in an amount per unit form (in zonisamide free acid) of from 25 mg to 600 mg, and
a statin also in a pharmaceutical composition comprising said statin in an amount per unit form of from 0.5 mg to 80 mg.

In said combination with 6-propylamino-4,5,6,7-tetrahydro-1,3-benzothiazole-2-amine Component (b) and at least one of fluoxetine, zonisamide or statin Component (c), domperidone Component (a) may also be in a fixed-dose combination (ab), (ac) or (abc), wherein the amount of domperidone Component (a) is equivalent to from 2 mg to 120 mg of domperidone base or from 2 mg to 40 mg; the amount of 6-propylamino-4,5,6,7-tetrahydro-1,3-benzothiazole-2-amine Component (b), is equivalent to from 0.125 mg to 3000 mg of pramipexole dihydrochloride monohydrate, including a (S)-enantiomer amount equivalent to from 0.125 mg to 45 mg of pramipexole dihydrochloride monohydrate; and the amount of the at least one synergistic agent Component (c) is selected from the group consisting of from 2 mg to 90 (in fluoxetine base) of fluoxetine or pharmaceutically acceptable salt or solvate thereof; from 25 mg to 600 (in zonisamide free acid) of zonisamide or pharmaceutically acceptable salt or solvate thereof; and from 0.5 mg to 80 mg of a statin.

A fixed-dose combination (ab) normally is in a pharmaceutical composition in dosage unit form comprising, as an active ingredient, said domperidone Component (a), in the above-illustrated amount per unit form; and, as a second active ingredient, 6-propylamino-4,5,6,7-tetrahydro-1,3-benzothiazole-2-amine Component (b), in an effective amount per unit form as illustrated in “The 6-propylamino-4,5,6,7-tetrahydro-1,3-benzothiazole-2-amine Component (b)” section below, in admixture with a pharmaceutical carrier or vehicle. Said fixed-dose combination is administered to a patient suffering from a PMND in combination with at least one synergistic agent Component (c) selected from the group consisting of fluoxetine, zonisamide and statins, also in a pharmaceutical composition in dosage unit form comprising at least one of said fluoxetine, zonisamide or statin in an effective amount per unit form as illustrated in “The synegistic agent Component (c)” section below, in admixture with a pharmaceutical carrier or vehicle.

A fixed-dose combination (ac) normally is in a pharmaceutical composition in dosage unit form comprising, as an active ingredient, said domperidone Component (a), in the above-illustrated amount per unit form; and, as a second active ingredient, at least one synergistic agent Component (c) selected from the group consisting of fluoxetine, zonisamide and statins, in an effective amount per unit form as illustrated in “The synegistic agent Component (c)” section below, in admixture with a pharmaceutical carrier or vehicle. Said fixed-dose combination is administered to a patient suffering from a PMND in combination with 6-propylamino-4,5,6,7-tetrahydro-1,3-benzothiazole-2-amine, also in a pharmaceutical composition in dosage unit form comprising said 6-propylamino-4,5,6,7-tetrahydro-1,3-benzothiazole-2-amine Component (b), in an effective amount per unit form as illustrated in “The 6-propylamino-4,5,6,7-tetrahydro-1,3-benzothiazole-2-amine Component (b)” section below, in admixture with a pharmaceutical carrier or vehicle. This fixed-dose combination (ac) has the advantage of allowing a flexibility in the dosage and in the mode of administration of the 6-propylamino-4,5,6,7-tetrahydro-1,3-benzothiazole-2-amine, in particular of pramipexole, in the treatment of a patient suffering from a PMND with the (ac/b) combination of the present invention.

In particular, a fixed-dose combination (ac) is in a pharmaceutical composition in dosage unit form comprising, as an active ingredient, said domperidone Component (a), in the above-illustrated amount per unit form; and, as a second active ingredient, statin Component (c), in an effective amount per unit form as illustrated in “The synergistic agent Component (c)” section below, in admixture with a pharmaceutical carrier or vehicle.

A fixed-dose combination (abc) normally is in a pharmaceutical composition in dosage unit form comprising, as an active ingredient, said domperidone Component (a), in the above-illustrated amount per unit form; as a second active ingredient, 6-propylamino-4,5,6,7-tetrahydro-1,3-benzothiazole-2-amine Component (b), in an effective amount per unit form as illustrated in “The 6-propylamino-4,5,6,7-tetrahydro-1,3-benzothiazole-2-amine Component (b)” section below; and, as a third active ingredient, at least one of fluoxetine, zonisamide or a statin Component (c), in an effective amount per unit form as illustrated in “The synegistic agent Component (c)” section below, in admixture with a pharmaceutical carrier or vehicle. This fixed-dose combination in this composition is useful in the treatment of a PMND in a patient in need of said treatment.

Finally, domperidone Component (a), in a pharmaceutical composition in dosage unit form comprising said domperidone, in the above-illustrated amount per unit form, is for use for the treatment of a PMND in a patient, in combination with a pharmaceutical composition Component (bc) comprising a pharmaceutical carrier or vehicle and a fixed-dose combination of 6-propylamino-4,5,6,7-tetrahydro-1,3-benzothiazole-2-amine in an effective amount per unit form as illustrated in “The 6-propylamino-4,5,6,7-tetrahydro-1,3-benzothiazole-2-amine Component (b)” section below; and at least one synergistic agent Component (c) selected from the group consisting of fluoxetine, zonisamide and statins, in an effective amount per unit form as illustrated in “The synegistic agent Component (c)” section below.

According to the present invention, domperidone may be used in the above pharmaceutical compositions in an amount per unit form within the above range, in particular in amount per unit form equivalent to from 2 mg to 120 mg, from 2 mg to 100 mg, from 2 mg to 80 mg, from 2 mg to 60 mg, from 2 mg to 40 mg, from 2 mg to 30 mg, or from 2 mg to 20 mg, normally from 10 mg to 100 mg, from 10 mg to 80 mg, from 10 mg to 60 mg or from 10 mg to 40 mg, from 10 mg to 30 mg, or from 10 mg to 20 mg of domperidone base. Using this compositions, domperidone is administered to a patient suffering from a PMND at a daily dose equivalent to from 4 mg to 120 mg, in particular equivalent to from 4 mg to 100 mg, from 4 mg to 80 mg, from 4 mg to 60 mg or from 4 mg to 40 mg, from 4 mg to 30 mg, or from 4 mg to 20 mg, normally from 10 mg to 100 mg, from 10 mg to 80 mg, from 10 mg to 60 mg, from 10 mg to 40 mg, from 10 mg to 30 mg, or from 10 mg to 20 mg of domperidone base.

The daily doses of the above Component (a) in said fixed dose combinations (ab), (ac), and (abc) are described above in this section.

The daily doses of the above Component (b) in said fixed dose combinations (ab), and (abc) are described below in “The 6-propylamino-4,5,6,7-tetrahydro-1,3-benzothiazole-2-amine Component (b)” section.

The daily doses of the above Component (c) in said fixed dose combinations (ac), and (abc) are described below in the “The synergistic agent Component (c)” section.

### The 6-propylamino-4,5,6,7-tetrahydro-1,3-benzothiazole-2-amine Component (b)

As set forth in the above Definitions, the 6-propylamino-4,5,6,7-tetrahydro-1,3-benzothiazole-2-amine Component (b) is selected from the group consisting of
pramipexole, i.e. (S)-6-propylamino-4,5,6,7-tetrahydro-1,3-benzothiazole-2-amine, and pharmaceutically acceptable salts and solvates thereof;
the racemate, i.e. (R,S)-6-propylamino-4,5,6,7-tetrahydro-1,3-benzothiazole-2-amine, and pharmaceutically acceptable salts and solvates thereof; and
a (S)/(R)-mixture, i.e. a mixture of (R)-6-propylamino-4,5,6,7-tetrahydro-1,3-benzothiazole-2-amine and (S)-6-propylamino-4,5,6,7-tetrahydro-1,3-benzothiazole-2-amine, normally in a pharmaceutical composition containing an effective amount of (S)-enantiomer, in admixture with a pharmaceutical carrier or vehicle.

The above Definitions also specify that the term “pramipexole” generally stands for (S)-6-propylamino-4,5,6,7-tetrahydro-1,3-benzothiazole-2-amine, as free base (pramipexole) or as pharmaceutically acceptable salts and solvates thereof, including pramipexole dihydrochloride monohydrate, their doses per unit form and their daily doses being expressed as equivalents of pramipexole dihydrochloride monohydrate, unless otherwise specified.

Pharmaceutically acceptable salts or solvates of pramipexole are also included in the term “pramipexole”.

Illustrative examples of pharmaceutically acceptable salts or solvates of 6-propylamino-4,5,6,7-tetrahydro-1,3-benzothiazole-2-amine are derived from inorganic or organic acids such as hydrochloric acid, hydrobromic acid, sulfuric acid, sulfamic acid, phosphoric acid, nitric acid, acetic acid, propionic acid, stearic acid, glycolic acid, oxalic acid, succinic acid, lactic acid, maleic acid, hydroxymaleic acid, fumaric acid, malic acid, tartaric acid, citric acid, ascorbic acid, phenylacetic acid, glutamic acid, benzoic acid, salicylic acid, 2-acetoxybenzoic acid, methanesulfonic acid, ethanesulfonic acid, 2-hydroxyethanesulfonic (isethionic) acid, p-toluenesulfonic acid, 2-naphthalenesulfonic acid, 4-amino-benzenesulfonic (sulfanilic) acid, 2,6-naphthalenedisulfonic acid, 1,5-naphthalenedisulfonic acid, and pamoic (embonic) acid. The solvation solvent is normally water.

In the case of pramipexole or pharmaceutically acceptable salt or solvate thereof, pramipexole dihydrochloride monohydrate, commercially available, is the preferred 6-propylamino-4,5,6,7-tetrahydro-1,3-benzothiazole-2-amine Component (b). Pramipexole base may be preferably used in some circumstances, for example in transdermal drug delivery systems. For example, stable pharmaceutical compositions comprising pramipexole dihydrochloride monohydrate that are disclosed in WO 2012/0140604 and in WO 2008/122638, the contents of each of which are incorporated herein by reference in their entirety, and sustained release compositions comprising pramipexole dihydrochloride monohydrate that are disclosed in U.S. Pat. No. 8,399,016, the contents of which are incorporated herein by reference in its entirety may be useful for use in combination with domperidone and with statin for the treatment of a PMND.

The racemate and pramipexole, described in U.S. Pat. No. 4,886,812, the contents of which are incorporated herein in their entirety by reference, are each a useful 6-propylamino-4,5,6,7-tetrahydro-1,3-benzothiazole-2-amine for the treatment of a PMND in combination with domperidone and with at least one of fluoxetine, zonisamide or a statin.

A (S)/(R)-mixture, i.e., a pharmaceutical composition comprising a therapeutically effective amount of (R)-6-propylamino-4,5,6,7-tetrahydro-1,3-benzothiazole-2-amine and a therapeutically effective amount of (S)-6-propylamino-4,5,6,7-tetrahydro-1,3-benzothiazole-2-amine, as disclosed in US 2008/0014259, the contents of which is incorporated herein in its entirety by reference, is also a useful Component (b) for the treatment of a PMND in combination with a domperidone Component (a) and with at least one of fluoxetine, zonisamide or a statin Component (c).

For its administration to a patient suffering from a PMND in combination with domperidone, as illustrated above in “The domperidone Component (a)” section, and with at least one of fluoxetine, zonisamide or a statin, as described in “The synegistic agent Component (c)” section below, the 6-propylamino-4,5,6,7-tetrahydro-1,3-benzothiazole-2-amine Component (b) is formulated in a pharmaceutical composition in dosage unit form comprising, as an active ingredient, said 6-propylamino-4,5,6,7-tetrahydro-1,3-benzothiazole-2-amine, in admixture with a pharmaceutical carrier or vehicle.

For this administration the 6-propylamino-4,5,6,7-tetrahydro-1,3-benzothiazole-2-amine Component (b) is formulated in a pharmaceutical composition in dosage unit form comprising said 6-propylamino-4,5,6,7-tetrahydro-1,3-benzothiazole-2-amine in an amount equivalent to from 0.125 mg to 3000 mg of pramipexole dihydrochloride monohydrate, including a (S)-enantiomer amount equivalent to from 0.125 mg to 45 mg, normally from 15 mg to 25 mg or from more than 20 mg to 25 mg of pramipexole dihydrochloride monohydrate, in admixture with a pharmaceutical carrier or vehicle. Said composition is administered to a patient in need of said treatment at a daily dose equivalent to from 0.375 mg to 3000 mg of pramipexole dihydrochloride monohydrate including a (S)-enantiomer daily dose equivalent to from 0.375 mg to 45 mg, normally from 7.5 mg to 25 mg, from 15 mg to 25 mg or from more than 20 mg to 25 mg of pramipexole dihydrochloride monohydrate, in combination with domperidone Component (a), at a daily dose of from 4 mg to 120 mg, and with at least one synergistic agent Component (c) selected from the group consisting of fluoxetine, at a daily dose (in fluoxetine base) of from 4 mg to 90 mg; zonisamide, at a daily dose of from 25 mg to 600 mg, normally from 200 mg to 600 mg; and a statin, at a daily dose of from 0.5 mg to 80 mg.

The effective 6-propylamino-4,5,6,7-tetrahydro-1,3-benzothiazole-2-amine Component (b) daily dose administered for treating a PMND in combination with domperidone Component (a) and with the Component (c) or in combination with the (ac) fixed-dose combination, as described in “The domperidone Component (a)” and in “The synergistic agent Component (c)” sections above, is equivalent to from 50 mg to 3000 mg of pramipexole dihydrochloride monohydrate, inclusive of a (S)-enantiomer daily dose equivalent to from 0.375 mg to 45 mg of pramipexole dihydrochloride monohydrate. This daily dose comprises pramipexole or (S)-enantiomer daily pediatric doses and doses used during the titration period.

The effective daily dose of pramipexole or (S)-enantiomer is a dose equivalent to at least the pramipexole dihydrochloride monohydrate approved daily dose for the treatment of PD. Said daily approved dose is from 0.375 mg to 4.5 mg. However, it is hereby specified that, according to the present invention, the combination of domperidone Component (a) and of at least one of fluoxetine, zonisamide or a statin with 6-propylamino-4,5,6,7-tetrahydro-1,3-benzothiazole-2-amine, including the (ac/b) combinations, allows the administration of (S)-6-propylamino-4,5,6,7-tetrahydro-1,3-benzothiazole-2-amine, as pramipexole or as (S)-enantiomer component present in the racemate or in a (S/R)-mixture, at daily doses as high as the pramipexole doses approved for the treatment of Parkinson's disease without any adverse effect, but also at daily doses that are higher and also much higher than said approved doses.

For example, the daily dose of pramipexole or a pharmaceutically acceptable salt thereof may be equivalent to from 1.1 times to 10 times, from 1.5 to 10 times, from 2.5 to 10 times, from 3 to 10 times, from more than 3.2 times to 10 times, normally from more than 3.2 to 8 times, from more than 3.2 to 6 times, or from more than 3.2 to 5 times higher than the pramipexole dihydrochloride monohydrate maximum recommended dose for the treatment of the symptoms of Parkinson's disease (such as motor symptoms).

For the treatment of a PMND in combination with domperidone, as described in “The domperidone Component (a)” section above, and with at least one of fluoxetine, zonisamide or statin Component (c), as described in “The synegistic agent Component (c)” below, pramipexole is administered to a patient in need of said treatment at a daily dose equivalent to from 0.375 mg to 45 mg of pramipexole dihydrochloride monohydrate. Said daily dose-range includes pediatric doses and low daily doses used during the titration period.

In particular, pramipexole or pharmaceutically acceptable salt or solvate thereof may be administered to a patient suffering from a PMND, including pediatric patients, at a daily dose equivalent to from 0.375 mg to 45 mg, from 1.5 mg to 45 mg, from more than 4.5 mg to 45 mg, from 5 mg to 45 mg, from more than 6 mg to 45 mg, from more than 10 mg to 45 mg, from 14.5 mg to 45 mg, or from 15 mg to 45 mg, normally from 0.375 mg to 20 mg, from more than 4.5 mg to 20 mg, from more than 6 mg to 20 mg, from 10 mg to 20 mg, from 13 mg to 20 mg, or from 15 mg to 20 mg of pramipexole dihydrochloride monohydrate, depending on the tolerability (in combination with domperidone and at least one of fluoxetine, zonisamide or a statin).

For its administration to a patient suffering from PMND, including pediatric patients, in combination with domperidone, as described in “The domperidone Component (a)” section above, and with at least one of fluoxetine, zonisamide, or a statin Component (c) as described in “The synegistic agent Component (c)” section below, the 6-propylamino-4,5,6,7-tetrahydro-1,3-benzothiazole-2-amine Component (b) is formulated in a pharmaceutical composition in dosage unit form comprising, as an active ingredient, said 6-propylamino-4,5,6,7-tetrahydro-1,3-benzothiazole-2-amine, in admixture with a pharmaceutical carrier or vehicle.

For this administration the 6-propylamino-4,5,6,7-tetrahydro-1,3-benzothiazole-2-amine Component (b) is formulated in a pharmaceutical composition in dosage unit form comprising said 6-propylamino-4,5,6,7-tetrahydro-1,3-benzothiazole-2-amine in an amount equivalent to from 0.125 mg to 3000 mg of pramipexole dihydrochloride monohydrate, including a (S)-enantiomer amount equivalent to from 0.125 mg to 45 mg, normally from 7.5 mg to 25 mg, from 15 mg to 25 mg or from more than 20 mg to 25 mg, of pramipexole dihydrochloride monohydrate, in admixture with a pharmaceutical carrier or vehicle. Said composition is administered to a patient in need of said treatment at a daily dose equivalent to from 0.375 mg to 3000 mg of pramipexole dihydrochloride monohydrate, including a (S)-enantiomer daily dose equivalent to from 0.375 mg to 45 mg, normally from 15 mg to 25 mg or from more than 20 mg to 25 mg, of pramipexole dihydrochloride monohydrate, in combination with a domperidone Component (a), at a daily dose equivalent to from 4 mg to 120 mg of domperidone base, and with at least one Component (c) selected from the group consisting of fluoxetine, at a daily dose (in fluozetine base) of from 4 mg to 90 mg, zonisamide, at a daily dose (in zonisamide free acid) of from 25 mg to 600 mg and a statin, at a daily dose of from 0.5 mg to 80 mg.

In particular, the 6-propylamino-4,5,6,7-tetrahydro-1,3-benzothiazole-2-amine is preferably selected from the group consisting of
(S)-6-propylamino-4,5,6,7-tetrahydro-1,3-benzothiazole-2-amine (INN: pramipexole) and pharmaceutically acceptable salts and solvates thereof, in particular its dihydrochloride monohydrate (USAN: pramipexole hydrochloride), in an amount per unit form equivalent to from 0.125 mg to 45 mg, normally from 7.5 mg to 25 mg, from 15 mg to 25 mg or from more than 20 mg to 25 mg of pramipexole dihydrochloride monohydrate;
(R,S)-6-propylamino-4,5,6,7-tetrahydro-1,3-benzothiazole-2-amine (the racemate) and pharmaceutically acceptable salts an solvates thereof, in an amount per unit form equivalent to from 0.25 mg to 90 mg, normally from from 15 mg to 50 mg, from 30 mg to 50 mg or from more than 40 mg to 50 mg of pramipexole dihydrochloride monohydrate (thus, obviously, including an amount per unit form of (S)-6-propylamino-4,5,6,7-tetrahydro-1,3-benzothiazole-2-amine equivalent to from 0.125 mg to 45 mg, normally from 7.5 mg to 25 mg, from 15 mg to 25 mg or from more than 20 mg to 25 mg of pramipexole dihydrochloride monohydrate, and an amount per unit form of (R)-6-propylamino-4,5,6,7-tetrahydro-1,3-benzothiazole-2-amine equivalent to from 0.125 mg to 45 mg, normally from 7.5 mg to 25 mg, from 15 mg to 25 mg or from more than 20 mg to 25 mg of pramipexole dihydrochloride monohydrate); and
a (R)/(S)-mixture, i.e. a pharmaceutical composition in dosage unit form comprising 6-propylamino-4,5,6,7-tetrahydro-1,3-benzothiazole-2-amine, in an amount per unit form equivalent to from 50 mg to 3000 mg, preferably to from 150 mg to 3000 mg, of pramipexole dihydrochloride monohydrate, said amount per unit form including a (S)-enantiomer amount equivalent to from 0.125 mg to 45 mg, normally from 7.5 mg to 25 mg, from 15 mg to 25 mg or from more than 20 mg to 25 mg (thus, obviously, said amount per unit form being constituted by an amount of (S)-enantiomer equivalent to from 0.125 mg to 45 mg, normally from 7.5 mg to 25 mg, from 15 mg to 25 mg or from more than 20 mg to 25 mg of pramipexole dihydrochloride monohydrate and by a (R)-6-propylamino-4,5,6,7-tetrahydro-1,3-benzothiazole-2-amine amount per unit form equivalent to from 50 mg, preferably from 150 mg, to 3000 mg (minus from 0.125 mg to 45 mg, normally minus from 7.5 mg to 25 mg, from 15 mg to 25 mg or from more than 20 mg to 25 mg) of pramipexole dihydrochloride monohydrate.

Normally, said pharmaceutical composition in dosage unit form comprises said 6-propylamino-4,5,6,7-tetrahydro-1,3-benzothiazole-2-amine in an amount equivalent to from 0.125 mg to 3000 mg of pramipexole dihydrochloride monohydrate, including a (S)-enantiomer amount equivalent to from 0.125 mg to 45 mg of pramipexole dihydrochloride monohydrate, in admixture with a pharmaceutical carrier or vehicle.

In the above pharmaceutical composition, said 6-propylamino-4,5,6,7-tetrahydro-1,3-benzothiazole-2-amine is in an amount equivalent to from 0.125 mg to 1500 mg of pramipexole dihydrochloride monohydrate, including a (S)-enantiomer amount of from 0.125 mg to 22.5 mg, normally from 7.5 mg to 22.5 mg or from more than 10 mg to 22.5 mg in an IR-formulation, or in an amount equivalent to from 0.375 mg to 3000 mg of pramipexole dihydrochloride monohydrate, including a (S)-enantiomer amount of from 0.375 mg to 45 mg or from more than 20 mg to 45, normally from 7.5 mg to 25 mg, from 15 mg to 25 mg or from more than 20 mg to 25 mg in an ER-formulation.

Said composition is administered to a patient in need of said treatment at a daily dose equivalent to from 0.375 mg to 3000 mg of pramipexole dihydrochloride monohydrate including a (S)-enantiomer daily dose equivalent to from 0.375 mg to 45 mg of pramipexole dihydrochloride monohydrate, in combination with domperidone Component (a), at a daily dose (in domperidone base) of from 4 mg to 120 mg and with at least one Component (c) selected from the group consisting of fluoxetine, at a daily dose equivalent to from 4 mg to 90 mg of fluoxetine base, zonisamide, at a daily dose of from 25 mg to 600 mg, and a statin, at a daily dose of from 0.5 mg to 80 mg.

In the above pharmaceutical composition, said 6-propylamino-4,5,6,7-tetrahydro-1,3-benzothiazole-2-amine is in an amount equivalent to from 0.125 mg to 1500 mg of pramipexole dihydrochloride monohydrate, including a (5)-enantiomer amount of from 0.125 mg to 22.5 mg, in an IR-formulation, or in an amount equivalent to from 0.375 mg to 3000 mg of pramipexole dihydrochloride monohydrate, including a (S)-enantiomer amount of from 0.375 mg to 45 mg or from more than 20 mg to 45 mg, in an ER-formulation.

According to a specific embodiment, the invention provides a pharmaceutical composition in dosage unit form comprising a 6-propylamino-4,5,6,7-tetrahydro-1,3-benzothiazole-2-amine selected from the group consisting of pramipexole and pharmaceutically acceptable salts and solvates thereof in an amount per unit form equivalent to from 7.25 mg to 45 mg, normally from 7.5 mg to 25 mg, up to from more than 20 mg to 45 mg, normally from 20.25 mg to 25 mg, of pramipexole dihydrochloride monohydrate, in admixture with a pharmaceutical carrier or vehicle.

More specifically, in the above composition, said pramipexole or pharmaceutically acceptable salt or solvate thereof is present either in an amount per unit form equivalent to from 7.25 mg to 22.5 mg, from 7.5 mg to 12.5 mg, or from more than 20 mg to 22.5 mg, normally from 20.25 mg to 22.5 mg of pramipexole dihydrochloride monohydrate in admixture with a pharmaceutical carrier or vehicle in an IR-formulation; or in an amount per unit form equivalent to from 15 mg to 45 mg, normally from 15 mg to 25 mg, or from more than 20 mg to 25 mg, normally from 20.25 mg to 25 mg of pramipexole dihydrochloride monohydrate in admixture with a pharmaceutical carrier or vehicle in an ER-formulation.

Pramipexole may be administered to a patient, including pediatric patients, suffering from a PMND at a daily dose of from 0.375 mg to 45 mg, depending on the tolerability (in combination with domperidone and at least one of fluoxetine, zonisamide or a statin). According to the present invention, the daily dose range of from 0.375 mg to 45 mg includes low doses to be administered to pediatric patient or during a titration period. More particularly, said daily dose range (in pramipexole dihydrochloride monohydrate) may be form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, and from 6.5 mg to 45 mg.

As set forth above, with domperidone, in combination with 6-propylamino-4,5,6,7-tetrahydro-1,3-benzothiazole-2-amine and with fluoxetine, zonisamide, or a statin, it is possible to treat a patient suffering from a PMND by maintaining a therapeutically effective 6-propylamino-4,5,6,7-tetrahydro-1,3-benzothiazole-2-amine daily dose with minimal adverse effect.

In order to provide concurrent administration of said domperidone and of said statin in combination with said 6-propylamino-4,5,6,7-tetrahydro-1,3-benzothiazole-2-amine, the invention provides fixed-dose combinations, in pharmaceutical compositions in dosage unit form comprising, as active ingredients, domperidone Component (a), at least one synergistic agent Component (c) selected from the group consisting of fluoxetine, zonisamide and statins; and, optionally, 6-propylamino-4,5,6,7-tetrahydro-1,3-benzothiazole-2-amine Component (b), in admixture with a pharmaceutical carrier or vehicle.

The domperidone/6-propylamino-4,5,6,7-tetrahydro-1,3-benzothiazole-2-amine/statin fixed-dose combinations are described in “The domperidone Component (a)” section above.

Thus, according to the present invention, 6-propylamino-4,5,6,7-tetrahydro-1,3-benzothiazole-2-amine Component (b), preferably pramipexole, is present in fixed-dose combinations,
in Component (ab) in a pharmaceutical composition comprising domperidone Component (a), in an amount per unit form as illustrated in “The domperidone Component (a)” section, and said 6-propylamino-4,5,6,7-tetrahydro-1,3-benzothiazole-2-amine Component (b), in a pharmaceutical composition comprising said 6-propylamino-4,5,6,7-tetrahydro-1,3-benzothiazole-2-amine Component (b), preferably pramipexole, in an amount per unit form as illustrated in this section, for the treatment of a PMND in combination with at least one synergistic agent Component (c) selected from the group consisting of fluoxetine, zonisamide and a statin, also in a pharmaceutical composition, in an amount per unit form as illustrated in “The synegistic agent Component (c)” section;
in Component (bc) in a pharmaceutical composition comprising said 6-propylamino-4,5,6,7-tetrahydro-1,3-benzothiazole-2-amine, preferably pramipexole, Component (b), in an amount per unit form as illustrated in this section, and at least one synergistic agent Component (c) selected from the group consisting of fluoxetine, zonisamide and statins, in an amount per unit form as illustrated in “The synergistic agent Component (c)” section below, for the treatment of a PMND in combination with domperidone Component (a), also in a pharmaceutical composition, in an amount per unit form as illustrated in “The domperidone Component (a)” section; or
in a fixed dose combination (abc) included in a pharmaceutical composition comprising domperidone Component (a), in an amount per unit form as illustrated in “The domperidone Component (a)” section, a 6-propylamino-4,5,6,7-tetrahydro-1,3-benzothiazole-2-amine, preferably pramipexole, Component (b), in an amount per unit form as illustrated in this section, and at least one synergistic agent selected from the group consisting of fluoxetine, zonisamide and statins Component (c), in an amount per unit form as illustrated in “The synegistic agent Component (c)” section.

In particular, in said combination, including fixed-dose combinations, with domperidone Component (a), and with the at lease one synergistic agent Component (c) selected from the group consisting of fluoxetine, zonisamide and statins, said 6-propylamino-4,5,6,7-tetrahydro-1,3-benzothiazole-2-amine Component (b) is pramipexole or a pharmaceutically acceptable salt thereof.

The amounts per unit form of domperidone in the domperidone/6-propylamino-4,5,6,7-tetrahydro-1,3-benzothiazole-2-amine/fluoxetine, zonisamide and/or statin fixed-dose combinations (ab), (ac) and (abc) are described in “The domperidone Component (a)” section above.

Among pramipexole and pharmaceutically acceptable salt or solvate thereof, pramipexole dihydrochloride monohydrate, commercially available, is preferred, but pramipexole base may be preferably used in some circumstances, for example in transdermal therapeutic systems. Stable pharmaceutical compositions comprising pramipexole dihydrochloride monohydrate are disclosed in WO 2012/0140604 and in WO 2008/122638, the contents of each of which are incorporated herein by reference in their entirety. Sustained release compositions comprising pramipexole dihydrochloride monohydrate, disclosed in U.S. Pat. No. 8,399,016, the contents of which are incorporated herein by reference in their entirety, may be useful for use in combination with domperidone Component (a) and with at least one of fluoxetine, zonisamide or a statin Component (c) for the treatment of a PMND.

For this use, in combination, including fixed dose combinations, with domperidone Component (a) and with at least one synergistic agent Component (c) selected from the group consisting of fluoxetine, zonisamide, or a statin, pramipexole Component (b) is formulated in a pharmaceutical composition in dosage unit form comprising said pramipexole in an amount per unit form equivalent to from 0.125 mg to 45 mg, normally from 15 mg to 25 mg or from more than 20 mg to 25 mg of pramipexole dihydrochloride monohydrate, in admixture with a pharmaceutical carrier or vehicle.

According to the present invention, preferred embodiments of said pharmaceutical composition including Component (b) comprise, as an active ingredient, pramipexole or a pharmaceutically acceptable salt thereof in an amount per unit form either
equivalent to from 0.125 mg to 30 mg, normally from 0.125 mg to 22.5 mg or from 7.25 mg to 22.5 mg of pramipexole dihydrochloride monohydrate, in an IR-formulation,
or in an amount per unit form equivalent to from 0.375 mg to 45 mg, normally from 1.5 to 45 mg, or from 15 mg to 45 mg of pramipexole dihydrochloride monohydrate, in an ER-formulation.

In particular, said pramipexole Component (b) is present in said composition in an amount per unit form equivalent to an amount-range per unit form selected from the group consisting of from 0.125 to 45 mg, from 1.5 mg to 45 mg, from 1.625 mg to 45 mg, from 3 mg to 45 mg, from more than 4.5 mg to 45 mg, from 4.8 mg to 45 mg, from more than 6 mg to 45 mg, and from more than 10 mg to 45 mg, and from 20.25 mg to 45 mg of pramipexole dihydrochloride monohydrate.

Preferably, said pramipexole is present in said composition in an amount per unit form equivalent to a range selected from the group consisting of from 14.5 mg to 45 mg, from 15 mg to 45 mg, from 15 mg to 40 mg, from 15 mg to 35 mg, from 15 mg to 30 mg, from 15 mg to 25 mg and from 20.25 to 25 mg of pramipexole dihydrochloride monohydrate, in admixture with a pharmaceutical carrier or vehicle.

As set forth above in this section, according to the present invention, pramipexole Component (b) may be administered to a patient, including pediatric patients, suffering from a PMND at a daily dose of from 0.375 mg to 45 mg, depending on the tolerability, in combination with domperidone Component (a) and at least one synergistic agent Component (c) selected from the group consisting of fluoxetine, zonisamide and statins. According to the present invention, the daily dose range of from 0.375 mg to 45 mg includes low doses to be administered during a titration period. More particularly, said daily dose range (in pramipexole dihydrochloride monohydrate) may be form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, and from 6.5 mg to 45 mg.

Preferably, said daily dose (in pramipexole dihydrochloride monohydrate) may be equivalent to a range selected from the group consisting of from more than 10 mg to 45 mg, from 14.5 mg to 45 mg, from 15 mg to 45 mg, from 15 mg to 35 mg, from 15 mg to 30 mg, from 15 mg to 25 mg and from more that 20 mg to 25 mg (in combination with domperidone and with at least one of fluoxetine, zonisamide, or a statin).

The daily doses of the above Component (a) in said fixed dose combinations (ab), (ac), and (abc) are described above in “The domperidone Component (a)” section.

The daily doses of the above Component (b) in said fixed dose combinations (ab), (bc), and (abc) are described above in this section.

The daily doses of the above Component (c) in said fixed dose combinations (ac), (bc), and (abc) is described below in “The synergistic agent Component (c)” section.

### The Synegistic Agent Component (c)

As set forth above, according to the present invention a combination (including fixed-dose combinations) of domperidone with at least one synergistic agent selected from the group consisting of fluoxetine, zonisamide and statins, allows for an increase of the therapeutic doses of 6-propylamino-4,5,6,7-tetrahydro-1,3-benzothiazole-2-amine, in particular of pramipexole, to consequently enable a better neuroprotective response in a patient suffering from a PMND such as PD and related disorders.

In particular, the above fixed-dose combinations include
a domperidone/fluoxetine or zonisamide or statin fixed-dose combination (ac),
a 6-propylamino-4,5,6,7-tetrahydro-1,3-benzothiazole-2-amine/fluoxetine, zonisamide and/or statin (in particular pramipexole/fluoxetine, zonisamide and/or statin) fixed-dose combination (bc), and
a domperidone/6-propylamino-4,5,6,7-tetrahydro-1,3-benzothiazole-2-amine/fluoxetine, zonisamide and/or statin (in particular a domperidone/6-propylamino-4,5,6,7-tetrahydro-1,3-benzothiazole-2-amine/fluoxetine , zonisamide and/or statin) fixed-dose combination (abc).

Herein, “fluoxetine” stands for 1-methylamino-3-phenyl-3-[4-(trifluoromethyl)phenoxy]propane as the free base or a salt or solvate thereof. Preferably, fluoxetine may be used as free base or as its hydrochloride salt.

Fluoxetine is a selective serotonin reuptake inhibitor (SSRI) antidepressant. It is currently used in the treatment of major depressive disorder, obsessive-compulsive disorder (OCD), bulimia nervosa, panic disorder, and premenstrual dysphoric disorder. When taken by mouth at recommended maintenance IR-doses (20 mg to 80 mg daily in 1 to 2 divided doses), or ER-dose (90 mg once weekly) by patients with these disorders, fluoxetine typically evinces a high degree of efficacy.

The mechanism by which fluoxetine benefits patients with psycho-affective disorders is generally considered to be linked to the drug's ability to augment CNS serotonin-mediated transmission. In addition, however, large fluoxetine doses in rodents have been shown to induce a significant increase in extracellular concentrations of norepinephrine and dopamine after acute systemic administration (Bymaster et al. 2002).

Fluoxetine augments levels of neurotrophic factors such as glial-derived neurotrophic factor (GDNF) and brain-derived neurotrophic factor (BDNF) and, in addition, the effects of fluoxetine in in vivo transgenic models of alpha-synucleinopathy have received careful investigative attention.

It has also been extensively reported to exhibit neuroprotective activity in various cellular and animal models of neurodegenerative disease (Ubhi K et al. 2012). For example, a laboratory study examined the effect of fluoxetine in the MBP1-hα-syntg mice, a model of MSA (Shults, et al. 2005).

Fluoxetine can protect against 6-OHDA (6-hydroxydopamine) (Suzuki, et al. 2010) and MPTP (1-methyl-4-phenyl-1,2,3,6-tetrahydropyridine) (Chung, et al. 2011)-induced damage in toxin-induced models of PD.

The term “effective daily dose of fluoxetine”, as used herein, refers to a daily dose of fluoxetine hydrochloride equivalent to from 4 mg to 90 mg of fluoxetine base. Said effective dose includes
(a) low daily doses of from 4 mg to less than 20 mg (in fluoxetine base) for use, in combination with a 5HT3-antagonist and/or a NK1-antagonist and 6-propylamino-4,5,6,7-tetrahydro-1,3-benzothiazole-2-amine, in pediatric patients and during the titration period of said 6-propylamino-4,5,6,7-tetrahydro-1,3-benzothiazole-2-amine;
(b) daily doses of from 20 mg to 90 mg, normally from 20 mg to 80 mg (in fluoxetine base), in an IR or ER formulation; and
(c) the fluoxetine daily dose released daily from the specific 90 mg ER-weekly preparation.

As set forth in the definitions, “fluoxetine” generally stands for the active principle per se, independently of the salt or solvate of said active principle, and “6-propylamino-4,5,6,7-tetrahydro-1,3-benzothiazole-2-amine” generally stands for the active principle per se, independently of the steric configuration and of the salt or solvate of said active principle.

In particular, the term “fluoxetine” includes the free base and pharmaceutically acceptable salts and solvates thereof, their doses per unit form or their daily doses being expressed as equivalents of fluoxetine base.

Pharmaceutically acceptable salts or solvates of fluoxetine are also included in the present invention. Illustrative examples of pharmaceutically acceptable salts of fluoxetine include acid addition salts with mineral or organic acids such as hydrochloric acid, hydrobromic acid, hydriodic acid, sulfuric acid, sulfamic acid, phosphoric acid, nitric acid, formic acid, acetic acid, propionic acid, stearic acid, glycolic acid, oxalic acid, malonic acid, succinic acid, lactic acid, maleic acid, hydroxymaleic acid, fumaric acid, malic acid, tartaric acid, citric acid, ascorbic acid, phenylacetic acid, carbonic acid, glutamic acid, benzoic acid, salicylic acid, 2-acetoxybenzoic acid, methanesulfonic acid, ethanesulfonic acid, 2-hydroxyethanesulfonic (isethionic) acid, p-toluenesulfonic acid, 2-naphthalenesulfonic acid, 4-amino-benzenesulfonic (sulfanilic) acid, 2,6-naphthalenedisulfonic acid, 1,5-naphthalenedisulfonic acid, aspartic acid and pamoic (embonic) acid. The solvation agent is generally water.

For its use for the treatment of a PMND in combination with domperidone Component (a) and with 6-propylamino-4,5,6,7-tetrahydro-1,3-benzothiazole-2-amine Component (b), fluoxetine Component (c) is formulated in a pharmaceutical composition in admixture with a pharmaceutical carrier or vehicle.

In the above composition, fluoxetine Component (c) is present in an amount equivalent to from 2 mg to 90 mg of fluoxetine hydrochloride.

In particular, the dose of fluoxetine per IR-unit form will be in an amount that is equivalent to from 2 mg to 45 mg or from 5 mg to 45 mg, normally from 2 mg to 40 mg or from 5 mg to 40 mg of fluoxetine base, depending on safety and tolerability, in combination with domperidone Component (a) and the 6-propylamino-4,5,6,7-tetrahydro-1,3-benzothiazole-2-amine Component (b). Preferably, said fluoxetine pharmaceutically acceptable salt is fluoxetine hydrochloride in the above IR-dose per unit form.

The dose per unit form of fluoxetine, or of a pharmaceutically acceptable salt or solvate thereof, in an ER-formulation, including slow-release compositions and TTDS, such as transdermal patches, will be in an amount (in fluoxetine base) of from 4 mg to 90 mg, normally from 20 mg to 90 mg, depending on safety and tolerability, in combination with domperidone Component (a) and the 6-propylamino-4,5,6,7-tetrahydro-1,3-benzothiazole-2-amine Component (b).

As set forth above, through the use of domperidone, in combination with 6-propylamino-4,5,6,7-tetrahydro-1,3-benzothiazole-2-amine and fluoxetine, it is possible to treat a patient suffering from a PMND by maintaining a therapeutic 6-propylamino-4,5,6,7-tetrahydro-1,3-benzothiazole-2-amine daily dose with minimal adverse effect.

In order to provide concurrent administration of said domperidone and of said fluoxetine, the invention provides fixed-dose combinations, in pharmaceutical compositions in dosage unit form comprising, as active ingredients, domperidone; and fluoxetine; and optionally 6-propylamino-4,5,6,7-tetrahydro-1,3-benzothiazole-2-amine, preferably pramipexole, in admixture with a pharmaceutical carrier or vehicle.

Thus, in said combination with domperidone and 6-propylamino-4,5,6,7-tetrahydro-1,3-benzothiazole, preferably pramipexole, fluoxetine may also be in a fixed-dose combination (ac), (bc) or (abc).

In particular, the above fixed-dose combinations include
a domperidone/fluoxetine fixed-dose combination (ac) comprising domperidone in an amount (in domperidone base) of from 2 mg to 120 mg and fluoxetine, in an amount (in fluoxetine base) of from 2 mg to 90 mg,
a 6-propylamino-4,5,6,7-tetrahydro-1,3-benzothiazole-2-amine/fluoxetine (in particular pramipexole/fluoxetine) fixed-dose combination (bc) comprising 6-propylamino-4,5,6,7-tetrahydro-1,3-benzothiazole-2-amine, in an amount of from 0.125 mg to 3000 mg, including a (S)-enantiomer amount equivalent to from 0.125 mg to 45 mg of pramipexole dihydrochloride monohydrate (in particular pramipexole in an amount equivalent to from 0.125 mg to 45 mg of pramipexole dihydrochloride monohydrate), and fluoxetine, in an amount (in fluoxetine base) of from 2 mg to 90 mg; and
a domperidone/6-propylamino-4,5,6,7-tetrahydro-1,3-benzothiazole-2-amine/fluoxetine (in particular a domperidone/pramipexole/fluoxetine) fixed-dose combination (abc) comprising domperidone in an amount (in domperidone base) of from 2 mg to 120 mg; 6-propylamino-4,5,6,7-tetrahydro-1,3-benzothiazole-2-amine, in an amount of from 0.125 mg to 3000 mg, including a (S)-enantiomer amount equivalent to from 0.125 mg to 45 mg of pramipexole dihydrochloride monohydrate (in particular pramipexole, in an amount equivalent to from 0.125 mg to 45 mg of pramipexole dihydrochloride monohydrate); and fluoxetine, in an amount (in fluoxetine base) of from 2 mg to 90 mg.

As stated in the definitions, zonisamide Component (c), is benzo[d]isoxazol-3-ylmethanesulfonamide

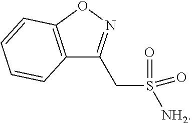 [SMILES: NS(=O)(=O)Cc1noc2ccccc12]

According to the present invention, zonisamide may be used, in the above acidic form or as an alkaline metal salt thereof, in particular as its sodium salt (zonisamide sodium), in combination with domperidone and with 6-propylamino-4,5,6,7-tetrahydro-1,3-benzothiazole-2-amine. Herein, unless otherwise specified, the term “zonisamide” includes its salts, but the doses per unit form and the daily doses refer to the free acid.

The effective daily dose of zonisamide is a dose at least as high as the zonisamide approved daily dose for its antiseizure indication. Said daily approved dose is from 200 mg to 600 mg.

For its use for the treatment of a PMND in combination with domperidone Component (a) and with 6-propylamino-4,5,6,7-tetrahydro-1,3-benzothiazole-2-amine Component (b), zonisamide Component (c) is formulated in a pharmaceutical composition in admixture with a pharmaceutical carrier or vehicle.

In the above composition, zonisamide Component (c) is present in an amount of from 25 mg to 600 mg.

In particular, the dose of zonisamide per IR-unit form will be in an amount of from 25 mg to 200 mg, depending on safety and tolerability, in combination with the domperidone Component (a) and pramipexole Component (b).

The dose of zonisamide per ER-unit form will be in an amount of from 25 mg to 600 mg, including low dose used in the pramipexole titration period, normally from 200 mg to 600 mg, depending on safety and 6-propylamino-4,5,6,7-tetrahydro-1,3-benzothiazole-2-amine, in combination with domperidone Component (a) and 6-propylamino-4,5,6,7-tetrahydro-1,3-benzothiazole-2-amine Component (b).

As set forth above, through the use of domperidone, in combination with 6-propylamino-4,5,6,7-tetrahydro-1,3-benzothiazole, preferably pramipexole, and zonisamide, it is possible to treat a patient suffering from a PMND by maintaining a therapeutically effective 6-propylamino-4,5,6,7-tetrahydro-1,3-benzothiazole-2-amine daily dose with minimal adverse effect.

In order to provide concurrent administration of said domperidone and of said zonisamide, the invention provides fixed-dose combinations, in pharmaceutical compositions in dosage unit form comprising, as active ingredients, domperidone; and zonisamide; and optionally 6-propylamino-4,5,6,7-tetrahydro-1,3-benzothiazole-2-amine, preferably pramipexole, in admixture with a pharmaceutical carrier or vehicle.

Thus, in said combination with domperidone and 6-propylamino-4,5,6,7-tetrahydro-1,3-benzothiazole, preferably pramipexole, zonisamide may also be in a fixed-dose combination (ac), (bc) or (abc).

In particular, the above fixed-dose combinations include
a domperidone/zonisamide fixed-dose combination (ac) comprising domperidone in an amount (in domperidone base) of from 2 mg to 120 mg and zonisamide, in an amount (in zonisamide free acid) of from 25 mg to 600 mg,
a 6-propylamino-4,5,6,7-tetrahydro-1,3-benzothiazole-2-amine/zonisamide (in particular pramipexole/zonisamide) fixed-dose combination (bc) comprising 6-propylamino-4,5,6,7-tetrahydro-1,3-benzothiazole-2-amine, in an amount of from 0.125 mg to 3000 mg, including a (S)-enantiomer amount equivalent to from 0.125 mg to 45 mg of pramipexole dihydrochloride monohydrate (in particular pramipexole in an amount equivalent to from 0.125 mg to 45 mg of pramipexole dihydrochloride monohydrate) and zonisamide, in an amount (in zonisamide free acid) of from 25 mg to 600 mg; and
a domperidone/6-propylamino-4,5,6,7-tetrahydro-1,3-benzothiazole-2-amine/zonisamide (in particular domperidone/pramipexole/zonisamide) fixed-dose combination (abc) comprising domperidone in an amount (in domperidone base) of from 2 mg to 120 mg; 6-propylamino-4,5,6,7-tetrahydro-1,3-benzothiazole-2-amine, in an amount of from 0.125 mg to 3000 mg, including a (S)-enantiomer amount equivalent to from 0.125 mg to 45 mg of pramipexole dihydrochloride monohydrate (in particular pramipexole in an amount equivalent to from 0.125 mg to 45 mg of pramipexole dihydrochloride monohydrate), and zonisamide, in an amount (in zonisamide free acid) of from 25 mg to 600 mg.

Prior to the present invention, the neuroprotective action of statins has not actually been evidenced in patients with a PMND and such action by statins alone would be expected to be minimal.

According to the present invention, a statin Component (c), in combination (including fixed-dose combinations) with domperidone, assures a safe treatment of PMND in patients in need of said treatment, in combination with a 6-propylamino-4,5,6,7-tetrahydro-1,3-benzothiazole-2-amine, in particular with pramipexole, with the intent of slowing or even arresting the progression of the disease.

Said statin may be selected from the group consisting of
(3R,5R)-7-[2-(4-fluorophenyl)-3-phenyl-4-(phenylcarbamoyl)-5-propan-2-ylpyrrol-1-yl]-3,5-dihydroxyheptanoic acid (atorvastatin) and pharmaceutically acceptable salts and solvates thereof, disclosed in U.S. Pat. No. 4,681,893 and, as the calcium salt thereof, in U.S. Pat. No. 5,273,995, the contents of both of which are incorporated herein in their entirety by reference;
(3R,5S,6E)-7-[4-(4-Fluorophenyl)-5-(methoxymethyl)-2,6-bis(propan-2-yl)pyridin-3-yl]-3,5-dihydroxyhept-6-enoic acid (cerivastatin) and pharmaceutically acceptable salts and solvates thereof, described in U.S. Pat. Nos. 5,006,530, 5,177,080 and 5,502,199, the contents of which are incorporated herein in their entirety by reference;
(4S,6R)-6-[(E)-2-[2-(4-fluoro-3-methylphenyl)-4,4,6,6-tetramethylcyclohexen-1-yl]ethenyl]-4-hydroxyoxan-2-one (dalvastatin), described in U.S. Pat. No. 4,863,957 and in EP 738510, the contents of both of which are incorporated herein in their entirety by reference;
[R*,S*-(E)]-(±)-7-[3-(4-fluorophenyl)-1-(1-methylethyl)-1H-indol-2-yl]-3,5-dihydroxy-6-heptenoic acid (fluindostatin) disclosed in European Patent Application Publication No. 363934 and, in a fixed-dose combination with amlodipine, in US 2002/0025981, the contents of both of which are incorporated herein in their entirety by reference.
(3R,5S,6E)-7-[3-(4-Fluorophenyl)-1-(propan-2-yl)-1H-indol-2-yl]-3,5-dihydroxyhept-6-enoic acid (fluvastatin), described in U.S. Pat. No. 4,739,073, the contents of which are incorporated herein in their entirety by reference;
(1S,3R,7S,8S,8aR)-8-{2-[(2R,4R)-4-Hydroxy-6-oxooxan-2-yl]ethyl}-3,7-dimethyl-1,2,3,7,8,8a-hexahydronaphthalen-1-yl (2S)-2-methylbutanoate (lovastatin), described in U.S. Pat. No. 4,231,938, the contents of which are incorporated herein in their entirety by reference;
(3R,5S,6E)-7-[2-Cyclopropyl-4-(4-fluorophenyl)quinolin-3-yl]-3,5-dihydroxyhept-6-enoic acid (pitavastatin) and pharmaceutically acceptable salts and solvates thereof, described in U.S. Pat. No. 5,011,930, the contents of which are incorporated herein in their entirety by reference;
(1S,7S,8S,8aR)-8-{2-[(2R,4R)-4-Hydroxy-6-oxotetrahydro-2H-pyran-2-yl]ethyl}-7-methyl-1,2,3,7,8,8a-hexahydronaphthalen-1-yl (2S)-2-methylbutanoate
(1S,7S,8S,8aR)-8-{2-[(2R,4R)-4-Hydroxy-6-oxotetrahydro-2H-pyran-2-yl]ethyl}-7-methyl-1,2,3,7,8,8a-hexahydronaphthalen-1-yl (2S)-2-methylbutanoate (mevastatin), disclosed in U.S. Pat. No. 3,983,140, the contents of which are incorporated herein in their entirety by reference;
(3R,5R)-3,5-dihydroxy-7-((1R,2S,6S,8R,8aR)-6-hydroxy-2-methyl-8-{[(2S)-2-methylbutanoyl]oxy}-1,2,6,7,8,8a-hexahydronaphthalen-1-yl)-heptanoic acid (pravastatin) and pharmaceutically acceptable salts and solvates thereof, described in U.S. Pat. No. 4,346,227, the contents of which are incorporated herein in their entirety by reference;
(1S,3R,7S,8S,8aR)-8-{2-[(2R,4R)-4-hydroxy-6-oxotetrahydro-2H-pyran-2-yl]ethyl}-3,7-dimethyl-1,2,3,7,8,8a-hexahydronaphthalen-1-yl 2,2-dimethylbutanoate; described in U.S. Pat. No. 4,444,784, the contents of which are incorporated herein in their entirety by reference;
(E,3R,5S)-7-[4-(4-fluorophenyl)-5-(methoxymethyl)-2,6-dipropan-2-yl-pyridin-3-yl]-3,5-dihydroxy-hept-6-enoic acid (rivastatin) and pharmaceutically acceptable salts and solvates thereof,
(3R,5S,6E)-7-[4-(4-Fluorophenyl)-2-(N-methylmethanesulfonamido)-6-(propan-2-yl)pyrimidin-5-yl]-3,5-dihydroxyhept-6-enoic acid (rosuvastatin) and pharmaceutically acceptable salts and solvates thereof, described in U.S. Pat. No. 5,260,440, the contents of which are incorporated herein in their entirety by reference.
butanoic acid, 2,2-dimethyl-,1,2,3,7,8,8a-hexahydro-3,7-dimethyl-8-[2-(tetrahydro-4-hydroxy-6-oxo-2H-pyran-2-yl)-ethyl]-1-naphthalenyl ester, [1S-[1α,3α,7β,8β(2S*,4S*),-8αβ]] (velostatin or synvinolin), disclosed in U.S. Pat. Nos. 4,448,784 and 4,450,171, both of which are incorporated herein by reference;
   and pharmaceutically acceptable salts and solvates and prodrugs of said statins. Esters of the above statins are also included in the context of the present invention.

According to the present invention, said statin is preferably selected from the group consisting of
atorvastatin, available in IR-tablets containing atorvastatin calcium trihydrate in an amount equivalent to 10 mg, 20 mg or 40 mg and 80 mg atorvastatin, to be administered once a day;
fluvastatin, available in IR capsules containing an amount of fluvastatin sodium, equivalent to 20 mg, 40 mg of fluvastatin, to be administered twice a day; and in 80 mg (in fluvastatin) ER tablets, to be administered once a day;
lovastatin, available in 20 mg and 40 mg IR-tablets to be administered twice a day and in 60 mg ER-tablets, to be administered once a day;
pitavastatin, available in tablets containing an amount of pitavastatin calcium equivalent to 1 mg, 2 mg and 4 mg of pitavastatin free acid;
pravastatin, available in tablets containing an amount of pravastatin sodium of 10 mg, 20 mg, 40 mg, and 80 mg, to be normally administered once a day;
rosuvastatin, available in tablets containing an 5 mg, 10 mg, 20 mg, and 40 mg of rosuvastatin calcium; and
simvastatin, available in 5 mg, 10 mg, 20 mg, 40 mg and 80 mg tablets, to be normally administered once a day.

A particularly preferred statin is lovastatin.

Chemically, these statins are characterized by a 3,5-dihydroxyheptane or 3,5-dihydroxyhept-6-ene carboxylic acid linked, via its 7-position, to a carbocyclic or heterocyclic structure. Thus, they can be in form of a lactone formed by loss of a H₂O between the carboxy group with the 5-hydroxy group of the 3,5-dihydroxyheptane carboxylic acid side-chain according to Scheme 1, wherein the steric configuration is not shown, and some of them are used in their lactone form.

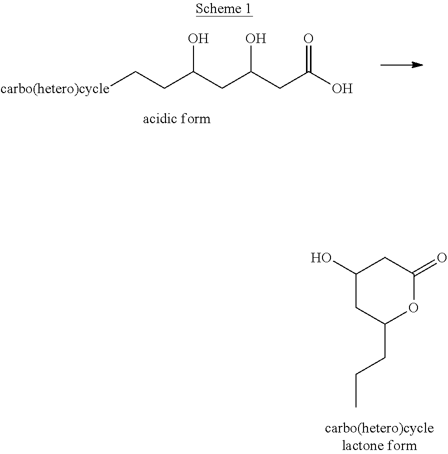

Both the acid and lactone forms of these acids are included in the family of statins of the present invention.

Herein, the expressions “salt or solvate thereof”, “salts or solvates thereof” and “salts and solvates thereof”, in reference to a statin in acidic form, indicate that the salt of said statin may be solvated with a solvent, normally water. Said salt normally is an alkaline metal salt or alkaline-earth metal salt, preferably sodium or calcium salt.

According to the present method (or use), the statin is administered to said patient at a daily dose that is from the half of the aforementioned daily dose approved for the treatment of dyslipidemia, up to the maximum daily dose approved for the treatment of dyslipidemia. Normally, said statin is administered at a daily dose of from 0.5 mg to 80 mg. Preferably, said daily dose is lower than the maximum approved daily dose of each of said statins.

Preferably, in the treatment of a patient suffering from a PMND, in combination with an effective daily dose of domperidone and with an effective daily dose of 6-propylamino-4,5,6,7-tetrahydro-1,3-benzothiazole-2-amine, a statin selected from the group consisting of
atorvastatin calcium trihydrate, administered to said patient at a daily dose equivalent to from 5 mg to 80 mg, normally from 5 mg to 60 mg of atorvastatin free acid;
fluvastatin sodium, administered to said patient at a daily dose equivalent to from 10 mg to 80 mg, normally from 10 mg to 60 mg of fluvastatin free acid;
lovastatin, administered to said patient at a daily dose of from 5 mg to 80 mg, normally from 5 mg to 60 mg;
pitavastatin calcium, administered to said patient at a daily dose equivalent to from 0.5 mg to 4 mg, normally from 0.5 mg to 3 mg of pitavastatin free acid;
pravastatin sodium, administered to said patient at a daily dose of from 2.5 mg to 60 mg, normally from 2.5 mg to 40 mg;
simvastatin, administered to said patient at a daily dose of from 2.5 mg to 40 mg, normally from 2.5 mg to 30 mg; and
rosuvastatin calcium, administered to said patient at a daily dose of from 2.5 mg to 40 mg, normally from 2.5 mg to 30 mg,
is particularly advantageous.

In order to be administered to a patient suffering from a PMND, the above statin is formulated in a pharmaceutical composition in dosage unit form comprising said statin in an amount per unit form of from 0.5 mg to 80 mg, in admixture with a pharmaceutical carrier or vehicle.

Said statin is preferably selected from the group consisting of
atorvastatine and pharmaceutically acceptable salts and solvates thereof, in an amount per unit form equivalent to from 5 mg to 80 mg, normally from 5 mg to 60 mg of atorvastatin free acid;
fluvastatin and pharmaceutically acceptable salts and solvates thereof, in an amount per unit form equivalent to from 10 mg to 80 mg, normally from 10 mg to 60 mg of fluvastatin free acid;
lovastatin, in an amount of from 2.5 mg to 80 mg or from 5 mg to 80 mg, normally from 5 mg to 60 mg or from 10 mg to 60 mg;
pitavastatin and pharmaceutically acceptable salts and solvates thereof, in an amount per unit form equivalent to from 0.5 mg to 4 mg, normally from 0.5 mg to 3 mg of pitavastatin free acid;
pravastatin and pharmaceutically acceptable salts and solvates thereof, in an amount per unit form equivalent to from 2.5 mg to 60 mg, normally from 2.5 mg to 40 mg of pravastatin sodium;
rosuvastatin and pharmaceutically acceptable salts and solvates thereof, in an amount per unit form equivalent to from 2.5 mg to 40 mg, normally from 2.5 mg to 30 mg of rosuvastatin calcium; and
simvastatin, in an amount per unit form of from 2.5 mg to 40 mg, normally from 2.5 mg to 30 mg.

As set forth above, according to the present invention, in said composition the above statins may be in a fixed-dose combination with domperidone, as Component (ac), said fixed-dose combination preferably comprising or consisting of
domperidone or a pharmaceutically acceptable salt or solvate thereof, in an amount equivalent to from 2 mg to 120 mg of domperidone base; and
a statin, in an amount of from 0.5 mg to 80 mg.

In order to provide concurrent administration of said domperidone and of said statin, the invention provides fixed-dose combinations, in pharmaceutical compositions in dosage unit form comprising, as active ingredients, domperidone; and a statin; and optionally 6-propylamino-4,5,6,7-tetrahydro-1,3-benzothiazole-2-amine, preferably pramipexole, in admixture with a pharmaceutical carrier or vehicle.

Thus, in said combination with domperidone and 6-propylamino-4,5,6,7-tetrahydro-1,3-benzothiazole, preferably pramipexole, a statin may also be in a fixed-dose combination (ac), (bc) or (abc).

In particular, the above fixed-dose combinations include
a domperidone/statin fixed-dose combination (ac) comprising domperidone in an amount (in domperidone base) of from 2 mg to 120 mg and a statin, in an amount of from 0.5 mg mg to 80 mg;
a 6-propylamino-4,5,6,7-tetrahydro-1,3-benzothiazole-2-amine/statin (in particular pramipexole/statin) fixed-dose combination (bc) comprising 6-propylamino-4,5,6,7-tetrahydro-1,3-benzothiazole-2-amine, in an amount of from 0.125 mg to 3000 mg, including a (S)-enantiomer amount equivalent to from 0.125 mg to 45 mg of pramipexole dihydrochloride monohydrate (in particular pramipexole in an amount equivalent to from 0.125 mg to 45 mg of pramipexole dihydrochloride monohydrate), and a statin, in an amount of from 0.5 mg to 80 mg; and
a domperidone/6-propylamino-4,5,6,7-tetrahydro-1,3-benzothiazole-2-amine/statin (in particular a domperidone/pramipexole/statin) fixed-dose combination (abc) comprising domperidone in an amount (in domperidone base) of from 2 mg to 120 mg; 6-propylamino-4,5,6,7-tetrahydro-1,3-benzothiazole-2-amine, in an amount of from 0.125 mg to 3000 mg, including a (S)-enantiomer amount equivalent to from 0.125 mg to 45 mg of pramipexole dihydrochloride monohydrate (in particular pramipexole, in an amount equivalent to from 0.125 mg to 45 mg of pramipexole dihydrochloride monohydrate); and a statin, in an amount of from 0.5 mg to 80 mg.

Preferably, in the above fixed-dose combinations, the statin is selected from the group consisting of atorvastatine and pharmaceutically acceptable salts and solvates thereof, in an amount equivalent to from 5 mg to 80 mg of atorvastatin free acid; fluvastatin and pharmaceutically acceptable salts and solvates thereof, in an amount equivalent to from 10 mg to 80 mg of fluvastatin free acid; lovastatin, in an amount of from 2.5 mg to 80 mg; pitavastatin and pharmaceutically acceptable salts and solvates thereof, in an amount equivalent to from 0.5 mg to 4 mg of pitavastatin free acid; pravastatin and pharmaceutically acceptable salts and solvates thereof, in an amount equivalent to from 2.5 mg to 60 mg of pravastatin sodium; rosuvastatin and pharmaceutically acceptable salts and solvates thereof, in an amount equivalent to from 2.5 mg to 40 mg of rosuvastatin calcium; and simvastatin, in an amount of from 2.5 mg to 40 mg.

Preferably, in the above fixed-dose combination, the statin is selected from the group consisting of atorvastatine and pharmaceutically acceptable salts and solvates thereof, in an amount per unit form equivalent to from 5 mg to 80 mg of atorvastatin free acid; fluvastatin and pharmaceutically acceptable salts and solvates thereof, in an amount per unit form equivalent to from 10 mg to 80 mg of fluvastatin free acid; lovastatin, in an amount of from 2.5 mg to 80 mg; pitavastatin and pharmaceutically acceptable salts and solvates thereof, in an amount per unit form equivalent to from 0.5 mg to 4 mg of pitavastatin free acid; pravastatin and pharmaceutically acceptable salts and solvates thereof, in an amount per unit form equivalent to from 2.5 mg to 60 mg of pravastatin sodium; rosuvastatin and pharmaceutically acceptable salts and solvates thereof, in an amount per unit form equivalent to from 2.5 mg to 40 mg of rosuvastatin calcium; and simvastatin, in an amount per unit form of from 2.5 mg to 40 mg.

Said fixed-dose combination may be (and normally is) in a pharmaceutical composition in dosage unit form comprising a domperidone Component (a), in an amount per unit form as illustrated in “The domperidone Component (a)” section and a statin Component (c), in an amount per unit form as described above.

In order to provide concurrent administration of said domperidone and of said statin, the invention provides fixed-dose combinations, in pharmaceutical compositions in dosage unit form comprising, as active ingredients, domperidone; and a statin; and optionally 6-propylamino-4,5,6,7-tetrahydro-1,3-benzothiazole-2-amine, preferably pramipexole, in admixture with a pharmaceutical carrier or vehicle.

In addition, in said combinations with domperidone Component (a) and, optionally, with the 6-propylamino-4,5,6,7-tetrahydro-1,3-benzothiazole-2-amine Component (b), preferably pramipexole, the statin Component (c) may also be present in a fixed-dose combination (ac), (bc) or (abc).

According to an embodiment, said combination comprises or consists of a pharmaceutical composition comprising a pharmaceutically acceptable carrier or vehicle and a fixed-dose combination of domperidone and a statin. This composition is useful or for use for the treatment of a PMND in a patient, in further combination with 6-propylamino-4,5,6,7-tetrahydro-1,3-benzothiazole-2-amine, normally with pramipexole.

A fixed-dose combination (ac) may also be in a pharmaceutical composition in dosage unit form comprising domperidone Component (a), in an effective amount per unit form as described above in “The domperidone Component (a)” section; and a statin Component (c), in an amount per unit form as described above in this section, in admixture with a pharmaceutical carrier or vehicle. This fixed-dose combination is administered to a patient suffering from a PMND in combination with 6-propylamino-4,5,6,7-tetrahydro-1,3-benzothiazole-2-amine Component (b), preferably pramipexole, also in a pharmaceutical composition in dosage unit form comprising an effective amount per unit form of said 6-propylamino-4,5,6,7-tetrahydro-1,3-benzothiazole-2-amine, preferably pramipexole, as described above in “The 6-propylamino-4,5,6,7-tetrahydro-1,3-benzothiazole-2-amine Component (b)” section, in admixture with a pharmaceutical carrier or vehicle.

As mentioned above, this Component (ac) of a domperidone/6-propylamino-4,5,6,7-tetrahydro-1,3-benzothiazole-2-amine/statin combination is particularly advantageous because it allows a flexibility in the dosage and in the mode of administration of the 6-propylamino-4,5,6,7-tetrahydro-1,3-benzothiazole-2-amine, Component (b), in particular of pramipexole.

According to a preferred embodiment, the invention provides a pharmaceutical composition comprising
(a) domperidone or a pharmaceutically acceptable salt or solvate thereof, in an amount equivalent to a range selected form the group consisting of from 2 mg to 120 mg, from 2 mg to 100 mg, from 2 mg to 80 mg, from 2 mg to 60 mg, from 2 mg to 40 mg, from 2 mg to 30 mg or from 2 mg to 20 mg of domperidone base; and
(c) a statin, in an amount per unit form of from 0.5 mg to 80 mg,
   in admixture with a pharmaceutical carrier or vehicle.

Normally, the above composition is in dosage unit form and the above amounts of domperidone Component (a) and of the statin Component (c) are per unit form.

According to an embodiment, the invention provides a pharmaceutical composition for use in the treatment of a patient suffering from a PMND, in combination with a 6-propylamino-4,5,6,7-tetrahydro-1,3-benzothiazole-2-amine, comprising a pharmaceutical carrier or vehicle. and a fixed-dose combination of domperidone and a statin.

A fixed-dose combination (bc) comprises may also be in a pharmaceutical composition in dosage unit form comprising said 6-propylamino-4,5,6,7-tetrahydro-1,3-benzothiazole-2-amine Component (b), preferably pramipexole, in an effective amount per unit form as illustrated above in “The 6-propylamino-4,5,6,7-tetrahydro-1,3-benzothiazole-2-amine Component (b)” section; and a statin Component (c), in an amount per unit form as illustrated above in this section, in admixture with a pharmaceutical carrier or vehicle. Said fixed-dose combination is administered to a patient suffering from a PMND in combination with the domperidone Component (a), also in a pharmaceutical composition in dosage unit form comprising said domperidone in an effective amount per unit form as illustrated above in “The domperidone Component (a)” section, in admixture with a pharmaceutical carrier or vehicle.

In particular, as Component (bc), the invention provides a pharmaceutical composition in dosage unit form comprising a 6-propylamino-4,5,6,7-tetrahydro-1,3-benzothiazole-2-amine selected from the group consisting of pramipexole and pharmaceutically acceptable salts and solvates thereof, in an amount per unit form equivalent to from more than 20 mg to 45 mg, normally from 20.25 mg to 45 mg of pramipexole dihydrochloride monohydrate, and a statin, in an amount per unit form of from 0.5 mg to 80 mg in admixture with a pharmaceutical carrier or vehicle.

More particularly, said statin is selected from the group consisting of lovastatin, in an amount per unit form of from 5 mg to 80 mg, normally from 10 mg to 60 mg, and rosuvastatin calcium, in an amount per unit form of from 2.5 mg to 40 mg.

According to another embodiment, said combination comprises or consists of a pharmaceutical composition comprising a pharmaceutically acceptable carrier or vehicle and a fixed-dose combination of domperidone, a 6-propylamino-4,5,6,7-tetrahydro-1,3-benzothiazole-2-amine, and a statin. This composition is for use for the treatment of a PMND in a patient. Said 6-propylamino-4,5,6,7-tetrahydro-1,3-benzothiazole-2-amine preferably is pramipexole.

A fixed-dose combination (abc) may further be in a pharmaceutical composition in dosage unit form comprising
domperidone Component (a), in an effective amount per unit form as illustrated above in “The domperidone Component (a)” section;
6-propylamino-4,5,6,7-tetrahydro-1,3-benzothiazole-2-amine, preferably pramipexole, Component (b), in an effective amount per unit form as illustrated above in “The 6-propylamino-4,5,6,7-tetrahydro-1,3-benzothiazole-2-amine Component (b)” section; and
a statin Component (c), in an effective amount per unit form as illustrated above in this section;
   in admixture with a pharmaceutical carrier or vehicle.

This fixed-dose combination is for the treatment of a PMND in a patient in need of said treatment.

A specific fixed-dose combination (abc) comprises or consists of
domperidone Component (a), in an amount (in domperidone base) of from 2 mg to 120 mg;
pramipexole Component (b), in an effective amount (in pramipexole dihydrochloride monohydrate) of from 7.5 mg to 25 mg; and
a statin Component (c), in an effective amount as illustrated in this section.

This specific fixed-dose combination (abc) is normally formulated in a pharmaceutical composition in dosage unit form comprising
domperidone Component (a), in an amount per unit form (in domperidone base) of from 2 mg to 120 mg;
pramipexole Component (b), in an effective amount per unit form (in pramipexole dihydrochloride monohydrate) of from 7.5 mg to 25 mg; and
a statin Component (c), in an effective amount per unit form as illustrated in this section,
   in admixture with a pharmaceutical carrier or vehicle.

The amounts per unit form and the daily doses of each of the above Component (a) and Component (b) in said fixed dose combinations (ac), (bc), and (abc), normally formulated in a pharmaceutical composition in admixture with a pharmaceutical carrier or vehicle, are illustrated in “The domperidone Component (a)” and, respectively, in “The 6-propylamino-4,5,6,7-tetrahydro-1,3-benzothiazole-2-amine Component (b)” sections above.

Said statin, as Component (c) or in Components (ac), (bc) or (abc), is selected from the group consisting of atorvastatin and pharmaceutically acceptable salts and solvates thereof, fluvastatin and pharmaceutically acceptable salts and solvates thereof, lovastatin, pitavastatin and pharmaceutically acceptable salts and solvates thereof, pravastatin and pharmaceutically acceptable salts and solvates thereof, rosuvastatin and pharmaceutically acceptable salts and solvates thereof; and simvastatin, each in an amount per unit form as described above in this section.

Preferably, said statin is selected from the group consisting of lovastatin, in an amount of from 5 mg to 80 mg, preferably from 5 mg to 60 mg; rosuvastatin and pharmaceutically acceptable salts thereof, in an amount equivalent to from 2.5 mg to 40 mg, preferably from 2.5 mg to 30 mg, of rosuvastatin calcium; and simvastatin, in an amount per unit form of from 2.5 mg to 40 mg, preferably from 2.5 mg to 30 mg.

According to an advantageous embodiment the domperidone, as Component (a) and in said fixed dose combinations (ab), (ac), and (abc) in this pharmaceutical composition, is selected from the group consisting of domperidone base, domperidone hydrochloride, domperidone maleate and domperidone succinate (1:1), in an amount equivalent to a range selected form the group consisting of from 10 mg to 100 mg, from 10 mg to 80 mg, from 10 mg to 60 mg, from 10 mg to 40, mg, from 10 mg to 30 mg or from 10 mg to 20 mg of domperidone base.

Normally, the above pharmaceutical composition Component (c), Component (ac), Component (bc) or composition (abc) is in dosage unit form and the above amount ranges are per unit form.

A composition Component (ac) may be used or is for use for the treatment of a PMND in a patient in need of said treatment, in combination with 6-propylamino-4,5,6,7-tetrahydro-1,3-benzothiazole-2-amine, preferably pramipexole, Component (b), also in a pharmaceutical composition, preferably in dosage unit form, comprising said 6-propylamino-4,5,6,7-tetrahydro-1,3-benzothiazole-2-amine, preferably pramipexole, in an amount as described in “The 6-propylamino-4,5,6,7-tetrahydro-1,3-benzothiazole-2-amine component (b)” section above.

More particularly, for said treatment, the statin Component (c), in a pharmaceutical composition Component (c), Component (ac), Component (bc) or composition (abc) as described above, is selected from the group consisting of
lovastatin, in an IR-dose/unit form of from 5 mg to 80 mg, normally from 20 mg to 40 mg, administered once or twice a day, or in an ER-dose/unit form of from 5 mg to 80 mg, normally from 20 mg to 60 mg, administered once a day; and
rosuvastatin, in an IR-dose/unit form equivalent to from 2.5 mg to 40 mg or from 2.5 mg to 30 mg of rosuvastatin calcium, administered once a day.

The daily doses of the above Component (a) in said fixed-dose combinations (ac), and (abc) are illustrated in “The domperidone Component (a)” section.

The daily doses of the above Component (b) in said fixed dose combinations (bc), and (abc) are illustrated in “The 6-propylamino-4,5,6,7-tetrahydro-1,3-benzothiazole-2-amine Component (b)” section above.

The daily dose of the above statin Component (c) in said fixed-dose combinations (ac), (bc), and (abc) is from 0.5 mg to 80 mg or from 2.5 mg to 80 mg. Said statin Component (c) daily doses include low dose for use in pediatric patients, and, in combination with domperidone and pramipexole low doses used in pediatric patients and during the titration period.

Normally, for the treatment of a PMND in combination with domperidone Component (a) and with 6-propylamino-4,5,6,7-tetrahydro-1,3-benzothiazole-2-amine Component (b), the synergistic agent Component (c) is selected from the group consisting of
fluoxetine, in the above pharmaceutical compositions, administered to a patient at daily IR-doses of from 4 mg to 90 mg, normally from 20 mg to 80 mg, or at a ER-dose of from 4 mg to 90 mg, normally from 20 mg to 90 mg once a day, or
zonisamide, in the above pharmaceutical compositions, administered to a patient at daily IR-doses/unit form of from 25 mg to 300 mg once or twice a day, or at a ER-dose of from 25 mg to 600 mg, normally from 200 mg to 600 mg, once a day, or
a statin, in a pharmaceutical composition as described above, administered to a patient at daily dose of from 0.5 mg to 80 mg or from 2.5 mg to 80 mg.

In preferred embodiments, fluoxetine Component (c) may be administered to a patient suffering from a PMND as the specific 90 mg ER-weekly preparation,
either in combination with domperidone Component (a) at a daily dose as illustrated in “The domperidone Component (a)” section and with a 6-propylamino-4,5,6,7-tetrahydro-1,3-benzothiazole-2-amine, preferably pramipexole, daily dose as illustrated in “The 6-propylamino-4,5,6,7-tetrahydro-1,3-benzothiazole-2-amine Component (b)” above section;
or in combination with a fixed-dose combination Component (ab) comprising daily doses of the above Component (a) in said fixed dose combinations (ab) as illustrated in “The domperidone Component (a)” above section; and daily doses of the above Component (b) in said fixed dose combinations (ab), as illustrated in “The 6-propylamino-4,5,6,7-tetrahydro-1,3-benzothiazole-2-amine Component (b)” above section.

In particular, for the treatment of a patient suffering from PMND in combination with a domperidone Component (a) and with 6-propylamino-4,5,6,7-tetrahydro-1,3-benzothiazole-2-amine Component (b), the statin Component (c), in a pharmaceutical composition as described above, is selected from the group consisting of lovastatin, in an amount per unit form of from 2.5 mg to 80 mg or from 5 mg to 80 mg, normally from 5 mg to 60 mg or from 10 mg to 60 mg; and rosuvastatin, in an amount per unit form equivalent to from 2.5 mg to 40 mg of rosuvastatin calcium.

More particularly, for said treatment, the statin Component (c), in a pharmaceutical composition as described above, is selected from the group consisting of
lovastatin, in an IR-dose/unit form of from 2.5 mg to 80 mg or from 5 mg to 80 mg, normally from 20 mg to 40 mg, administered once or twice a day, or in an ER-dose/unit form of from 5 mg to 80 mg, normally from 20 mg to 60 mg, administered once a day; and
rosuvastatin, in an IR-dose/unit form equivalent to from 2.5 mg to 40 mg or from 2.5 mg to 30 mg of rosuvastatin calcium, administered once a day.

In preferred embodiments, lovastatin or rosuvastatin calcium Component (c) may be administered to a patient suffering from a PMND, in the above doses per unit form,
in combination with a domperidone Component (a) at a daily dose as described in “The domperidone Component (a)” above section and with a 6-propylamino-4,5,6,7-tetrahydro-1,3-benzothiazole-2-amine, preferably pramipexole, daily dose as described in “The 6-propylamino-4,5,6,7-tetrahydro-1,3-benzothiazole-2-amine Component (b)” above section;
in combination with a fixed-dose combination Component (ab) comprising daily doses of the above Component (a) in said fixed dose combinations (ab) as illustrated in “The domperidone Component (a)” above section; and daily doses of the above Component (b) in said fixed dose combinations (ab), as illustrated in “The 6-propylamino-4,5,6,7-tetrahydro-1,3-benzothiazole-2-amine Component (b)” above section; or
in a fixed-dose combination (abc) with a domperidone Component (a) at a daily dose as described in “The domperidone Component (a)” above section; and with a 6-propylamino-4,5,6,7-tetrahydro-1,3-benzothiazole-2-amine, at a daily dose as described in “The 6-propylamino-4,5,6,7-tetrahydro-1,3-benzothiazole-2-amine Component (b)” above section.

A particularly preferred fixed-dose combination (abc) is in a pharmaceutical composition in dosage unit form comprising domperidone, in an amount per unit from equivalent to from 2 mg to 120 mg of domperidone base; pramipexole or a pharmaceutically acceptable salt or solvate thereof, in an amount per unit form equivalent to from 0.125 mg to 45 mg, normally from 7.5 mg to 25 mg, from 15 mg to 25 mg or from 20.25 mg to 25 mg of pramipexole dihydrochloride monohydrate; and a statin is selected from the group consisting of lovastatin, in an amount per unit form of from 2.5 mg to 80 mg or from 5 mg to 80 mg (normally from 5 mg to 60 mg or from 10 mg to 60 mg), and rosuvastatin calcium, in an amount per unit form of from 2.5 mg to 40 mg.

### First Aspect of the Invention

The present invention provides a method for treating a protein misfolding neurodegenerative disease (“PMND”) in a patient, which comprises administering to said patient in need of said treatment domperidone, in combination with 6-propylamino-4,5,6,7-tetrahydro-1,3-benzothiazole-2-amine and with at least one synergistic agent selected from the group consisting of fluoxetine, zonisamide, and statins.

According to this first aspect, the invention includes a method for safely treating a PMND in a patient suffering from this disorder with domperidone and with at least one synergistic agent selected from the group consisting of fluoxetine, zonisamide, and statins, by concurrently or sequentially administering to said patient a 6-propylamino-4,5,6,7-tetrahydro-1,3-benzothiazole-2-amine, preferably pramipexole.

In particular, the present invention provides a method for safely treating a PMND in a patient in need of said treatment, which comprises administering to said patient an effective daily dose of domperidone or a pharmaceutically acceptable salt thereof Component (a), in combination with an effective daily dose of 6-propylamino-4,5,6,7-tetrahydro-1,3-benzothiazole-2-amine or a pharmaceutically acceptable salt thereof Component (b), and with at least one of an effective daily dose of fluoxetine, an effective daily dose of zonisamide, or an effective daily dose of a statin Component (c).

Herein the expressions “salt or solvate thereof”, “salts or solvates thereof” and “salts and solvates thereof”, referred to domperidone, to 6-propylamino-4,5,6,7-tetrahydro-1,3-benzothiazole-2-amine and, respectively, to fluoxetine, mean that said domperidone said 6-propylamino-4,5,6,7-tetrahydro-1,3-benzothiazole-2-amine and, respectively, said fluoxetine may be in the form of the free base or of a pharmaceutically acceptable acid addition salt thereof that may be solvated with a solvent, normally water.

In said method, the domperidone Component (a) is formulated in a pharmaceutical composition in dosage unit form comprising, as an active ingredient, said domperidone or pharmaceutically acceptable salt or solvate thereof, in an amount per unit form equivalent to from 2 mg to 120 mg of domperidone base, in admixture with a pharmaceutical carrier or vehicle. Said domperidone, in this composition, is administered at a daily dose equivalent to from 4 mg to 120 mg of domperidone base, for the treatment of a PMND in a patient in combination with 6-propylamino-4,5,6,7-tetrahydro-1,3-benzothiazole-2-amine Component (b) and with at least one synergistic agent Component (c) selected from the group consisting of fluoxetine, zonisamide and statins, each formulated in a pharmaceutical composition.

Said composition is administered for the treatment of a PMND in a patient in combination with 6-propylamino-4,5,6,7-tetrahydro-1,3-benzothiazole-2-amine Component (b), also in a pharmaceutical composition comprising said Component (b) in an amount per unit form equivalent to from 0.125 mg to 3000 mg of pramipexole dihydrochloride monohydrate, including a (S)-enantiomer amount per unit form equivalent to from 0.125 mg to 45 mg, normally from 7.5 mg to 25 mg, from 15 mg to 25 mg or from more than 20 mg to 25 mg of pramipexole dihydrochloride monohydrate, and with at least one synergistic agent Component (c) selected from the group consisting of fluoxetine, in an amount per unit from equivalent to from 2 mg to 90 mg of fluoxetine base, in admixture with a pharmaceutical carrier or vehicle; zonisamide, in an amount per unit from equivalent to from 25 mg to 600 mg of zonisamide free acid, in admixture with a pharmaceutical carrier or vehicle; and a statin, in an amount per unit from equivalent to from 0.5 mg to 80 mg, in admixture with a pharmaceutical carrier or vehicle.

The above pharmaceutically compositions comprising Component (b) and Component (c) are administered concurrently or sequentially with the above Component (a).

In particular, in said method,
(a) the domperidone Component (a) is formulated in a pharmaceutical composition in dosage unit form comprising, as an active ingredient, said domperidone, in an amount per unit form equivalent to from 2 mg to 60 mg of domperidone base, in admixture with a pharmaceutical carrier in IR-formulation; or in an amount per unit form equivalent to from 4 mg to 120 mg of domperidone base, in admixture with a pharmaceutical carrier in ER-formulation;
(b) the 6-propylamino-4,5,6,7-tetrahydro-1,3-benzothiazole-2-amine Component (b) is formulated in a pharmaceutical composition in dosage unit form comprising, as an active ingredient, said 6-propylamino-4,5,6,7-tetrahydro-1,3-benzothiazole-2-amine
   in an amount per unit form equivalent to from 0.125 mg to 1500 mg of pramipexole dihydrochloride monohydrate including a (S)-enantiomer amount equivalent to from 0.125 mg to 22.5 mg, normally from 7.5 mg to 12.5 mg or from more than 10 mg to 12.5 mg of pramipexole dihydrochloride monohydrate, in admixture with a pharmaceutical carrier or vehicle in an IR-formulation; or
   in an amount equivalent to from 0.375 mg to 3000 mg of pramipexole dihydrochloride monohydrate including a (S)-enantiomer amount equivalent to from 0.375 mg to 45 mg, normally from 15 mg to 25 mg or from more than 20 mg to 25 mg of pramipexole dihydrochloride monohydrate, in admixture with a pharmaceutical carrier in an ER-formulation; and
(c) the at least one synergistic agent is selected from the group consisting of
   (i) fluoxetine, formulated in a pharmaceutical composition in dosage unit form comprising, as an active ingredient, said fluoxetine
      in an amount per unit form equivalent to from 2 mg to 45 mg, normally from 2 mg to 40 mg, of fluoxetine base, in admixture with a pharmaceutical carrier or vehicle in an IR-formulation; or
      in an amount per unit form equivalent to from 4 mg to 90 mg, normally from 4 mg to 80 mg, of fluoxetine base in admixture with a pharmaceutical carrier or vehicle in an ER-formulation for a once a day administration; or
      in an amount of 90 mg, as the specific 90 mg ER-weekly preparation for once a week administration,
   (ii) zonisamide, formulated in a pharmaceutical composition in dosage unit form comprising, as an active ingredient, said zonisamide
      in an amount per unit form equivalent to from 25 mg to 200 mg, normally from 25 mg to 100 mg of zonisamide free acid, in admixture with a pharmaceutical carrier or vehicle in an IR-formulation; or
      in an amount per unit form equivalent to from 25 mg to 600 mg, normally from 200 mg to 600 mg, of zonisamide free acid in admixture with a pharmaceutical carrier or vehicle in an ER-formulation, and
   (iii) a statin, formulated in a pharmaceutical composition in dosage unit form comprising, as an active ingredient, said statin, in an amount per unit form of from 0.5 mg to 80 mg in IR-formulation or ER-formulation.

In the method of the present invention, the domperidone Component (a) active ingredient is present in the above compositions in an amount per unit form equivalent to a range selected from the group consisting of from 2 mg to 120 mg, from 2 mg to 100 mg, from 2 mg to 80 mg, from 2 mg to 60 mg, from 2 mg to 40 mg, from 2 mg to 30 mg or from 2 mg to 20 mg, normally from 10 mg to 120 mg, from 10 mg to 100 mg, from 10 mg to 80 mg, from 10 mg to 60 mg, from 10 mg to 40 mg, from 10 mg to 30 mg or from 10 mg to 20 mg of domperidone base. Preferably, said domperidone is selected from the group consisting of domperidone base, domperidone maleate and domperidone succinate (1:1).

In said method, the 6-propylamino-4,5,6,7-tetrahydro-1,3-benzothiazole-2-amine Component (b) in said composition is present in an amount per unit form equivalent to from 0.125 mg to 3000 mg of pramipexole dihydrochloride monohydrate, inclusive of a (S)-enantiomer amount per unit form equivalent to from 0.125 mg to 45 mg, normally from 7.5 mg to 25 mg, from 15 mg to 25 mg or from more than 20 mg to 25 mg of pramipexole dihydrochloride monohydrate.

In said above method, the 6-propylamino-4,5,6,7-tetrahydro-1,3-benzothiazole-2-amine Component (b) in said composition is advantageously selected from the group consisting of pramipexole, in an amount per unit form equivalent to from 0.125 mg to 45 mg of pramipexole dihydrochloride monohydrate; racemic 6-propylamino-4,5,6,7-tetrahydro-1,3-benzothiazole-2-amine, in an amount per unit form equivalent to from 0.25 mg to 90 mg of pramipexole dihydrochloride monohydrate; and a (R/S)-mixture, in an amount per unit form equivalent to from 50 mg to 3000 mg of pramipexole dihydrochloride monohydrate, inclusive of a (S)-enantiomer amount per unit form equivalent to from 0.125 mg to 45 mg of pramipexole dihydrochloride monohydrate.

In said above method, the at least one synergistic agent Component (c) in said composition is selected from the group consisting of fluoxetine, in an amount per unit form equivalent to from 2 mg to 90 mg, normally from 2 mg to 80 mg, of fluoxetine base; zonisamide, in an amount per unit form of from 25 mg to 600 mg; and a statin, in an amount per unit form of from 0.5 mg to 80 mg, normally from 2.5 mg to 80 mg.

The preferred 6-propylamino-4,5,6,7-tetrahydro-1,3-benzothiazole-2-amine Component (b) is pramipexole or a pharmaceutically acceptable salt or solvate thereof, in an amount per unit form (including low amounts per unit form used in pediatric patients and in the titration period) equivalent to a range selected from the group consisting of from 0.125 mg to 45 mg, from 1.5 mg to 45 mg, from 1.625 mg to 45 mg, from 3 mg to 45 mg, from more than 4.5 mg to 45 mg, from 4.8 mg to 45 mg, from more than 6 mg to 45 mg, from 7.5 mg to 45 mg, from more than 10 mg to 45 mg, from 14.5 mg to 45 mg, from 15 mg to 45 mg, from 15 mg to 40 mg, from 15 mg to 35 mg and from 15 mg to 30 mg, normally in a range selected from the group consisting of from 7.5 mg to 25 mg, from 15 mg to 25 mg, from more than 20 mg to 25 mg, and from 20.25 mg to 25 mg of pramipexole dihydrochloride monohydrate.

Specific pramipexole amounts per unit form are described above in “The 6-propylamino-4,5,6,7-tetrahydro-1,3-benzothiazole-2-amine Component (b)” section and below, in the “Specific embodiments” section.

In particular, this first aspect of the present invention provides a method for treating a PMND in a patient in need of said treatment, which comprises administering to said patient a domperidone Component (a), at a daily dose (in domperidone base) of from 4 mg to 120 mg, in combination with a daily dose of said 6-propylamino-4,5,6,7-tetrahydro-1,3-benzothiazole-2-amine Component (b) equivalent to from 0.375 mg to 3000 mg of pramipexole dihydrochloride monohydrate, including a (S)-enantiomer amount daily dose equivalent to from 0.375 mg to 45 mg, normally from 15 mg to 25 mg or from more than 20 mg to 25 mg of pramipexole dihydrochloride monohydrate; and with at least one of a daily dose of fluoxetine Component (c) equivalent to from 4 mg to 90 mg of fluoxetine base, or with a daily dose of zonisamide Component (c) of from 25 mg to 600 mg, or with a daily dose of statin Component (c) of from 0.5 mg to 80 mg.

More particularly, according to this first aspect, the invention provides a method for treating a PMND in a patient in need of said treatment, which comprises administering to said patient a domperidone Component (a) selected from the group consisting of domperidone base, domperidone hydrochloride, domperidone maleate and domperidone succinate (1:1), at a daily dose equivalent to a range selected from the group consisting of from 4 mg to 120 mg, from 4 mg to 100 mg, from 4 mg to 80 mg, from 4 mg to 60 mg, from 4 mg to 40 mg, from 4 mg to 30 mg or from 4 mg to 20 mg of domperidone base, in combination with a pramipexole Component (b) daily dose equivalent to from 0.375 mg to 45 mg, from 14.5 mg to 45 mg or from more than 20 mg to 45 mg, normally from 15 mg to 25 mg or from 20.25 mg to 25 mg of pramipexole dihydrochloride monohydrate, and with at least one synergistic agent Component (c) selected from the group consisting of fluoxetine, at a daily dose equivalent to from 4 mg to 90 mg of fluoxetine base; zonisamide, at a daily dose of from 25 mg to 600 mg, and a statin, at a daily dose of from 0.5 mg to 80 mg, normally from 2.5 mg to 80 mg. Preferably, said domperidone daily dose is equivalent to a range selected from the group consisting of from 20 mg to 100 mg, from 20 mg to 80 mg, from 20 mg to 60 mg, from 20 mg to 40 mg, from 20 mg to 30 mg of domperidone base.

In combination with the above daily doses of Component (a) and Component (b), the synergistic agent Component (c) may also be fluoxetine, administered once a week in the specific 90 mg ER-weekly preparation.

In the treatment of a PMND, a domperidone/pramipexole/fluoxetine zoniamide and/or statin combination may also be selected from the group consisting of
a domperidone Component (a), in an amount per unit form (in domperidone base) of from 2 mg to 120 mg, for the treatment of a PMND in a patient in combination with a pharmaceutical composition Component (bc) comprising 6-propylamino-4,5,6,7-tetrahydro-1,3-benzothiazole-2-amine, in an amount per unit form equivalent to from 0.125 mg to 3000 mg of pramipexole dihydrochloride monohydrate, including a (S)-enantiomer amount equivalent to from 0.125 mg to 45 mg, normally from 7.5 mg to 25 mg, from 15 mg to 25 mg or from more than 20 mg to 25 mg of pramipexole dihydrochloride dihydrate and at least one synergistic agent selected from the group consisting of fluoxetine, in an amount per unit form equivalent to from 2 mg to 90 mg of fluoxetine base to be administered once or twice a day, zonisamide, in an amount per unit form of from 25 mg to 600 mg to be administered once or twice a day, and a statin, in an amount per unit form equivalent to from 0.5 mg to 80 mg to be administered once or twice a day;
a Component (ab), comprising domperidone, in an amount per unit form of from 2 mg to 120 mg; and 6-propylamino-4,5,6,7-tetrahydro-1,3-benzothiazole-2-amine, in an amount per unit form equivalent to from 0.125 mg to 3000 mg of pramipexole dihydrochloride monohydrate, including a (S)-enantiomer amount equivalent to from 0.125 mg to 45 mg, normally from 7.5 mg to 25 mg, from 15 mg to 25 mg or from more than 20 mg to 25 mg of pramipexole dihydrochloride monohydrate, for the treatment of a PMND in a patient in combination with at least one synergistic agent Component (c) selected from the group consisting of fluoxetine, in an amount per unit form equivalent to from 2 mg to 90 mg of fluoxetine base, to be administered once or twice per day or in the specific 90 mg ER-weekly preparation (to be administered once a week), zonisamide, in an amount per unit form equivalent to from 25 mg to 600 mg, or a statin, in an amount per unit form of from 0.5 mg to 80 mg, to be administered once or twice a day; and
a Component (ac), comprising domperidone, in an amount per unit form equivalent to from 2 mg to 120 mg; and at least one synergistic agent selected from the group consisting of fluoxetine, in an amount per unit form equivalent to from 2 mg to 90 mg of fluoxetine base, zonisamide, in an amount per unit form equivalent to from 25 mg to 600 mg, and a statin, in an amount per unit form of from 0.5 mg to 80 mg, for the treatment of a PMND in a patient in combination with Component (b), comprising 6-propylamino-4,5,6,7-tetrahydro-1,3-benzothiazole-2-amine, in an amount per unit form equivalent to from 0.125 mg to 3000 mg of pramipexole dihydrochloride monohydrate, including a (S)-enantiomer amount equivalent to from 0.125 mg to 45 mg of pramipexole dihydrochloride monohydrate, in particular equivalent to from 0.125 mg to 22.5 mg, normally from 7.5 mg to 12.5 mg or from more than 10 mg to 22.5 mg of pramipexole dihydrochloride monohydrate in IR-formulation to be administered twice a day, or from 0.375 mg to 45 mg, normally from 15 mg to 25 mg or from more than 20 mg to 25 mg of pramipexole dihydrochloride monohydrate in an ER-formulation to be administered once a day,
   each of said Component (a), Component (b), Component (c), Component (ab), Component (ac) and Component (bc) being or consisting of a pharmaceutical composition in dosage unit form wherein the active ingredients are in admixture with a pharmaceutical carrier or vehicle.

Specific amounts per unit form and daily doses of the above domperidone, 6-propylamino-4,5,6,7-tetrahydro-1,3-benzothiazole-2-amine and at least one synergistic agent in the pharmaceutical composition Component (a), Component (b), Component (c), Component (ab), Component (ac) and Component (bc) are illustrated in the “Specific embodiments” section below.

The fixed-dose combination (ac), and its use in the treatment of a PMND, as described in detail in “The domperidone Component (a) section above, is an advantageous embodiment of this first aspect of the invention.

Preferably, in said compositions, the 6-propylamino-4,5,6,7-tetrahydro-1,3-benzothiazole-2-amine is pramipexole or a pharmaceutically acceptable salt or solvate thereof, in an amount per unit form equivalent to from 0.125 mg to 45 mg, from 7.5 mg to 45 mg, from 14.5 mg to 45 mg or from more than 20 mg to 45 mg, normally from 7.5 mg to 25 mg, from 15 mg to 25 mg or from 20.25 mg to 25 mg of pramipexole dihydrochloride monohydrate.

### Second Aspect of the Invention

According to a second aspect, the invention provides the “use of domperidone for the preparation of a medicament” (or “domperidone for use”) for the treatment of a PMND in a patient in need of said treatment, in combination with an effective daily dose of 6-propylamino-4,5,6,7-tetrahydro-1,3-benzothiazole-2-amine and and an effective daily dose of at least one synergistic agent selected from the group consisting of fluoxetin, zonisamide, and statins.

According to an advantageous embodiment, the invention provides a pharmaceutical composition for use in treatment of a PMND in a patient, in combination with 6-propylamino-4,5,6,7-tetrahydro-1,3-benzothiazole-2-amine, comprising a pharmaceutical carrier or vehicle and a fixed-dose combination of domperidone and at least one synergistic agent selected from the group consisting of fluoxetine, zonisamide, and statins.

The invention also provides a pharmaceutical composition for use in treatment of a PMND in a patient, comprising a pharmaceutically acceptable carrier or vehicle and a fixed dose combination of domperidone, a 6-propylamino-4,5,6,7-tetrahydro-1,3-benzothiazole-2-amine and at least one synergistic agent selected from the group consisting of fluoxetine, zonisamide, and statins.

According to this second aspect, the invention provides a pharmaceutical combination comprising
(a) domperidone or a pharmaceutically acceptable salt or solvate thereof, in an amount per unit form of from 2 mg to 120 mg, to be administered at a daily dose of from 4 mg to 120 mg;
(b) a 6-propylamino-4,5,6,7-tetrahydro-1,3-benzothiazole-2-amine or a pharmaceutically acceptable salt or solvate thereof, in an amount per unit form equivalent to from 0.125 mg to 3000 mg of pramipexole dihydrochloride monohydrate, inclusive of a (S)-enantiomer amount per unit form equivalent to from 0.125 mg to 45 mg of pramipexole dihydrochloride monohydrate, to be administered at a daily dose of from 0.375 mg to 3000 mg of pramipexole dihydrochloride monohydrate, inclusive of a (S)-enantiomer daily dose equivalent to from 0.375 mg to 45 mg of pramipexole dihydrochloride monohydrate and
(c) at least one synergistic agent selected from the group consisting of fluoxetine, at a dose per unit form that is equivalent to from 2 mg to 90 mg of fluoxetine base, to be administered at a daily dose (in fluoxetine base) of from 4 mg to 90 mg; zonisamide, at a dose per unit form or daily dose (in zonisamide free acid) of from 25 mg to 600 mg to be administered at a daily dose of from 25 mg to 600 mg, normally from 200 mg to 600 mg; and a statin, in an amount per unit form of from 0.5 mg to 80 mg, to be administered at a daily dose of from 0.5 mg to 80 mg,
   for use for the treatment of a PMND.

For this use, said domperidone is formulated in a pharmaceutical composition in dosage unit form comprising, as an active ingredient, said domperidone, in admixture with a pharmaceutical carrier or vehicle. This pharmaceutical composition is indicated for the treatment of a PMND in a patient in need of said treatment, in combination with 6-propylamino-4,5,6,7-tetrahydro-1,3-benzothiazole-2-amine and with at least one of at least one of fluxetine, zonisamide, or a statin, each at an effective daily dose.

In said pharmaceutical composition, said domperidone is in admixture with a pharmaceutical carrier or vehicle and formulated in a dosage unit forms for oral, intravenous, transcutaneous, and/or transdermal administration, as described in “The formulations” section below.

According to this second aspect of the present invention, domperidone, as described in “The domperidone Component (a)” section, may be used as an active ingredient of said pharmaceutical composition for the treatment of a PMND in combination with any 6-propylamino-4,5,6,7-tetrahydro-1,3-benzothiazole-2-amine as described in “The 6-propylamino-4,5,6,7-tetrahydro-1,3-benzothiazole-2-amine Component (b)” section; and with at least one of fluoxetine, zonisamide, or statin Component (c) as described in “The synegistic agent Component (c)” section.

In particular, the invention provides domperidone for use in the treatment of a PMND in a patient, in combination with pramipexole and fluoxetine or zonisamide, or a statin.

For this use, domperidone or a pharmaceutically acceptable salt or solvate thereof is formulated in a pharmaceutical composition comprising, as an active ingredient, said domperidone in an amount per unit form equivalent to from 2 mg to 120 mg, of domperidone base, in admixture with a pharmaceutical carrier or vehicle. Normally, said composition is in dosage unit form.

This composition is indicated for the treatment of a PMND in a patient, at a domperidone daily dose equivalent to from 4 mg to 120 mg of domperidone base, in combination with 6-propylamino-4,5,6,7-tetrahydro-1,3-benzothiazole-2-amine daily dose equivalent to from 0.375 mg to 3000 mg of pramipexole dihydrochloride monohydrate, inclusive of a (S)-enantiomer daily dose equivalent to from 0.375 mg to 45 mg, normally from 15 mg to 25 mg or from more than 20 mg to 25 mg of pramipexole dihydrochloride monohydrate, and with at least one synergistic agent selected from the group consisting of fluoxetine, at a daily dose (in fluoxetine base) of from 4 mg to 90 mg; zonisamide, at a daily dose (in zonisamide free acid) of from 25 mg to 600 mg, normally from 200 mg to 600 mg; and a statin, at a daily dose of from 0.5 mg to 80 mg.

More particularly, this second aspect of the present invention provides domperidone Component (a), at a daily dose (in domperidone base) of from 4 mg to 120 mg, for use for the treatment of a PMND in a patient in need of said treatment in combination with a daily dose of said 6-propylamino-4,5,6,7-tetrahydro-1,3-benzothiazole-2-amine Component (b) equivalent to from 0.375 mg to 3000 mg of pramipexole dihydrochloride monohydrate, inclusive of a (S)-enantiomer daily dose equivalent to from 0.375 mg to 45 mg, normally from 15 mg to 25 mg or from more than 20 mg to 25 mg of pramipexole dihydrochloride monohydrate; and with at least one synergistic agent Component (c) selected from the group consisting of fluoxetine, at a daily dose (in fluoxetine nase) of from 4 mg to 90 mg or in the specific 90 mg ER-weekly preparation; zonisamide at a daily dose of from 25 mg to 600 mg, normally from 200 mg to 600 mg; and a statin, at daily dose of from 0.5 mg to 80 mg.

These daily doses of 6-propylamino-4,5,6,7-tetrahydro-1,3-benzothiazole-2-amine include low pramipexole or (S)-enantiomer daily doses useful for the administration, in combination with domperidone and with at least one of fluoxetine, zonisamide or a statin, to pediatric patients or during the titration period. In the second case, at the end of said titration period, the medicament thus manufactured enables the safe intake of pramipexole daily doses never heretofore attained (without the combination with the domperidone and with at least one of fluoxetine, zonisamide or a statin) as described in “The 6-propylamino-4,5,6,7-tetrahydro-1,3-benzothiazole-2-amine Component (b)” section.

Said 6-propylamino-4,5,6,7-tetrahydro-1,3-benzothiazole-2-amine may also be the racemate, at a daily dose equivalent to from 0.75 mg to 90 mg, normally from 30 mg to 50 mg or from more than 40 mg to 50 mg of pramipexole dihydrochloride monohydrate.

Preferably, said 6-propylamino-4,5,6,7-tetrahydro-1,3-benzothiazole-2-amine is pramipexole or pharmaceutically acceptable salt or solvate thereof, at a daily dose equivalent to from 0.375 mg to 45 mg, normally from 15 mg to 25 mg or from more than 20 mg to 25 mg of pramipexole dihydrochloride monohydrate.

According to an embodiment, said domperidone Component (a) is for use in combination with 6-propylamino-4,5,6,7-tetrahydro-1,3-benzothiazole-2-amine Component (b), as a (R)/(S)-mixture, at a daily dose of from 50 to 3000 mg, including a (S)-enantiomer daily dose equivalent to from 0.375 mg to 45 mg, normally from 15 mg to 25 mg or from more than 20 mg to 25 mg of pramipexole dihydrochloride monohydrate, and with fluoxetine, at a daily dose equivalent to from 4 mg to 90 mg of fluoxetine base; or with the fluoxetine specific 90 mg ER-weekly preparation.

Said 6-propylamino-4,5,6,7-tetrahydro-1,3-benzothiazole-2-amine may also be the racemate, at a daily dose equivalent to from 0.75 mg to 90 mg, normally from 30 mg to 50 mg or from more than 40 mg to 50 mg of pramipexole dihydrochloride monohydrate.

Preferably, said 6-propylamino-4,5,6,7-tetrahydro-1,3-benzothiazole-2-amine is pramipexole or pharmaceutically acceptable salt or solvate thereof, at a daily dose equivalent to from 0.375 mg to 45 mg, normally from 15 mg to 25 mg, from 20.25 mg to 25 mg, or from more than 20 mg to 45 mg of pramipexole dihydrochloride monohydrate.

For the treatment of a PMND, the domperidone, the 6-propylamino-4,5,6,7-tetrahydro-1,3-benzothiazole-2-amine and the synergistic agent are each formulated in a pharmaceutical composition, normally in dosage unit form, comprising said domperidone and, respectively, said 6-propylamino-4,5,6,7-tetrahydro-1,3-benzothiazole-2-amine and said at least one synergistic agent, each in admixture with a pharmaceutical carrier or vehicle; and are concurrently or sequentially administered at the above daily doses to a patient in need of said treatment.

In general, domperidone is formulated in a pharmaceutical composition in dosage unit form comprising said domperidone in an amount per unit form equivalent to from 2 mg to 120 mg of domperidone base.

The 6-propylamino-4,5,6,7-tetrahydro-1,3-benzothiazole-2-amine is formulated in a pharmaceutical composition in dosage unit form comprising said 6-propylamino-4,5,6,7-tetrahydro-1,3-benzothiazole-2-amine in an amount per unit of from equivalent to from 0.125 mg to 3000 mg of pramipexole dihydrochloride monohydrate, including an (S)-enantiomer amount per unit form equivalent to from 0.125 mg to 45 mg, normally from 7.5 mg to 25 mg, from 15 mg to 25 mg or from more than 20 mg to 25 mg of pramipexole dihydrochloride monohydrate, in admixture with a pharmaceutical carrier or vehicle.

Fluoxetine is formulated in a pharmaceutical composition in dosage unit form comprising said fluoxetine in an amount per unit form equivalent to from 2 mg to 90 mg, normally from 2 mg to 80 mg, of fluoxetine base, in admixture with a pharmaceutical carrier or vehicle It may also be formulated in the specific 90 mg ER-weekly preparation.

Zonisamide is formulated in a pharmaceutical composition in dosage unit form comprising said zonisamide in an amount per unit form equivalent to from 25 mg to 600 mg of zonisamide free acid.

The statin is formulated in a pharmaceutical composition in dosage unit form comprising said statin in an amount per unit form of from 0.5 mg to 80 mg, in admixture with a pharmaceutical carrier or vehicle.

Preferably, in the above combination, said 6-propylamino-4,5,6,7-tetrahydro-1,3-benzothiazole-2-amine is selected from the group consisting of pramipexole, in an amount per unit form equivalent to from 0.125 mg to 45 mg, normally from 7.5 mg to 25 mg, from 15 mg to 25 mg or from more than 20 mg to 25 mg of pramipexole dihydrochloride monohydrate, racemic 6-propylamino-4,5,6,7-tetrahydro-1,3-benzothiazole-2-amine, in an amount per unit form equivalent to from 0.25 mg to 90 mg, normally from 15 mg to 50 mg, from 30 mg to 50 mg or from more than 40 mg to 50 mg of pramipexole dihydrochloride monohydrate, and a (R)/(S)-mixture, in an amount per unit form of from equivalent to from 50 mg to 3000 mg of pramipexole dihydrochloride monohydrate, including a (S)-enantiomer amount of from 0.125 mg to 45 mg normally from 7.5 mg to 25 mg, from 15 mg to 25 mg or from more than 20 mg to 25 mg of pramipexole dihydrochloride monohydrate.

In particular, according to this second aspect, the invention provides a pharmaceutical combination selected from the group consisting of domperidone or a pharmaceutically acceptable salt or solvate thereof Component (a), in an amount per unit form (in domperidone base) of from 2 mg to 120 mg, in admixture with a pharmaceutical carrier or vehicle; in combination with a 6-propylamino-4,5,6,7-tetrahydro-1,3-benzothiazole-2-amine Component (b) selected from the group consisting of pramipexole and pharmaceutically acceptable salts thereof, at a daily dose equivalent to from 0.375 mg to 45 mg, normally from 15 mg to 25 mg, from more than 20 mg to 25 mg or from 20.25 mg to 25 mg of pramipexole dihydrochloride monohydrate; and with at least one synergistic agent Component (c) selected from the group consisting of fluoxetine, at a daily dose equivalent to from 4 mg to 90 mg of fluoxetine base, or in the specific 90 mg ER-weekly preparation; zonisamide, at a daily dose equivalent to from 25 mg to to 600 mg, normally from 200 mg to 600 mg; and a statin, at a daily dose of from 0.5 mg to 80 mg.

Said domperidone is preferably selected from the group consisting of domperidone base, domperidone hydrochloride, domperidone maleate and domperidone succinate (1:1).

According to this second aspect, the invention provides the use of domperidone Component (a) for the preparation of a medicament (or a domperidone for use) for the treatment of a PMND in a patient, in combination, including fixed-dose combinations, with 6-propylamino-4,5,6,7-tetrahydro-1,3-benzothiazole-2-amine Component (b) and with at least one synergistic agent selected from the group consisting of fluoxetine, zonisamide and statins Component (c). Said medicament comprises said domperidone, alone or together with a 6-propylamino-4,5,6,7-tetrahydro-1,3-benzothiazole-2-amine or with fluoxetine, zonisamide or a statin in a Component (ab) and Component (ac), respectively.

Said combination comprises a Component selected from the group consisting of
domperidone Component (a) in an amount equivalent to from 2 mg to 120 mg of domperidone base, for use in the treatment of a PMND in a patient in combination with a Component (bc) comprising 6-propylamino-4,5,6,7-tetrahydro-1,3-benzothiazole-2-amine, in an amount per unit form equivalent to from 0.125 mg to 3000 mg of pramipexole dihydrochloride monohydrate, including a (S)-enantiomer amount per unit form equivalent to from 0.125 mg to 45 mg, normally from 7.5 mg to 25 mg, from 15 mg to 25 mg or from more than 20 mg to 25 mg of pramipexole dihydrochloride monohydrate, and at least one synergistic agent selected from the group consisting of fluoxetine in an amount per unit form (in fluoxetine base) of from 2 mg to 90 mg, zonisamide, in an amount per unit form (in zonisamide free acid) of from 25 mg to to 600 mg, and a statin, in an amount per unit form of from 0.5 mg to 80 mg;
a Component (ab), comprising domperidone, in an amount per unit form equivalent to from 2 mg to 120 mg of domperidone base; and a 6-propylamino-4,5,6,7-tetrahydro-1,3-benzothiazole-2-amine, in an amount per unit form equivalent to from 0.125 mg to 3000 mg of pramipexole dihydrochloride monohydrate including a (S)-enantiomer amount per unit from equivalent to from 0.125 mg to 45 mg, normally from 7.5 mg to 25 mg, from 15 mg to 25 mg or from more than 20 mg to 25 mg of pramipexole dihydrochloride monohydrate, for the treatment of a PMND in a patient, in combination with Component (c) at least one synergistic agent selected from the group consisting of fluoxetine, in an amount per unit form (in fluoxetine base) of from 2 mg to 90 mg, zonisamide, in an amount per unit form (in zonisamide free acid) of from 25 mg to to 600 mg; and a statin, in an amount per unit form of from 0.5 mg to 80 mg; and
a Component (ac), comprising a domperidone, in an amount per unit form equivalent to from 2 mg to 120 mg of domperidone base, and at least one synergistic agent selected from the group consisting of fluoxetine in an amount per unit form (in fluoxetine base) of from 2 mg to 90 mg, zonisamide in an amount per unit form (in zonisamide free acid) of from 25 mg to to 600 mg, and a statin, in an amount per unit form of from 0.5 mg to 80 mg, for the treatment of a PMND in a patient in need of said treatment, in combination with Component (b), comprising a 6-propylamino-4,5,6,7-tetrahydro-1,3-benzothiazole-2-amine, in an amount per unit form equivalent to from 0.125 mg to 3000 mg of pramipexole dihydrochloride monohydrate, including a (S)-enantiomer amount per unit form equivalent to from 0.125 mg to 45 mg, normally from 7.5 mg to 25 mg, from 15 mg to 25 mg or from more than 20 mg to 25 mg of pramipexole dihydrochloride monohydrate,
   each of said Component (a), Component (b), Component (c), Component (ab), Component (ac) and Component (bc) being or consisting of a pharmaceutical composition in dosage unit form wherein the active ingredients are in admixture with a pharmaceutical carrier or vehicle.

The aforementioned pharmaceutical composition, Component (ac), comprising domperidone or a pharmaceutically acceptable salt or solvate thereof, in an amount per unit form equivalent to from 2 mg to 120 mg of domperidone base, and at least one synergistic agent selected from the group consisting of fluoxetine in an amount per unit form (in fluoxetine base) of from 2 mg to 90 mg, zonisamide in an amount per unit form (in zonisamide free acid) of from 25 mg to to 600 mg, and a statin in an amount per unit form of from 0.5 mg to 80 mg, is a particular embodiment of the present invention.

Specific amounts per unit form and daily doses of the above domperidone, 6-propylamino-4,5,6,7-tetrahydro-1,3-benzothiazole-2-amine and fluoxetine, zonisamide or statin in the pharmaceutical compositions Component (a), Component (b), Component (c), Component (ab), Component (ac) and Component (bc) are described in the “Specific embodiments” section below.

In particular, the domperidone Component (a), in the above compositions, is administered at a daily dose equivalent to a range selected from the group consisting of from 4 mg to 120 mg, from 4 mg to 100 mg, from 4 mg to 80 mg, from 4 mg to 60 mg, from 4 mg to 40 mg, from 4 mg to 30 mg or from 4 mg to 20 mg, normally from 10 mg to 120 mg, from 10 mg to 100 mg, from 10 mg to 80 mg, from 10 mg to 60 mg, from 10 mg to 40 mg, from 10 mg to 30 mg or from 10 mg to 20 mg of domperidone base. Preferably, said domperidone is selected from the group consisting of domperidone base, domperidone hydrochloride, domperidone maleate and domperidone succinate (1:1).

According to an advantageous embodiment of this second aspect, the invention also provides the Component (ac), as a pharmaceutical composition for use in treatment of a PMND in a patient, comprising a pharmaceutically acceptable carrier or vehicle and a fixed-dose combination of domperidone, and a statin, to be administered to said patient in further combination with a 6-propylamino-4,5,6,7-tetrahydro-1,3-benzothiazole-2-amine Component (b).

Said Component (ac) comprises or consists of a pharmaceutical composition in dosage unit form comprising
(a) domperidone, in an amount per unit form equivalent to from 2 mg to 120 mg of domperidone base; and
(c) a statin selected from the group consisting of atorvastatine and pharmaceutically acceptable salts and solvates thereof, in an amount per unit form equivalent to from 5 mg to 80 mg of atorvastatin free acid; fluvastatin and pharmaceutically acceptable salts and solvates thereof, in an amount per unit form equivalent to from 10 mg to 80 mg of fluvastatin free acid; lovastatin, in an amount per unit form of from 5 mg to 80 mg; pitavastatin and pharmaceutically acceptable salts and solvates thereof, in an amount per unit form equivalent to from 0.5 mg to 4 mg of pitavastatin free acid; pravastatin and pharmaceutically acceptable salts and solvates thereof, in an amount per unit form equivalent to from 2.5 mg to 60 mg of pravastatin sodium; rosuvastatin and pharmaceutically acceptable salts and solvates thereof, in an amount per unit form equivalent to from 2.5 mg to 40 mg of rosuvastatin calcium; and simvastatin, in an amount per unit form of from 2.5 mg to 40 mg,
   in admixture with a pharmaceutical carrier or vehicle. This composition is useful for the treatment of a PMND in combination with 6-propylamino-4,5,6,7-tetrahydro-1,3-benzothiazole-2-amine.

In the above pharmaceutical composition, the amount per unit form may be equivalent to a range selected from the group consisting of from 2 mg to 120 mg, 2 mg to 100 mg, from 2 mg to 80 mg, from 2 mg to 60 mg, from 2 mg to 40 mg, from 2 mg to 30 mg and from 2 mg to 20 mg, normally from 10 mg to 120 mg, from 10 mg to 100 mg, from 10 mg to 80 mg, from 10 mg to 60 mg, from 10 mg to 40 mg, from 10 mg to 30 mg and from 10 mg to 20 mg of domperidone base.

The advantage of Component (ac) of a domperidone/6-propylamino-4,5,6,7-tetrahydro-1,3-benzothiazole-2-amine/fluoxetine, zonisamide or statin combination is the possibility of assuring a flexibility in the dosage and in the mode of administration of the 6-propylamino-4,5,6,7-tetrahydro-1,3-benzothiazole-2-amine, in particular of pramipexole.

In a particular advantageous Component (ac), said domperidone is selected from the group consisting of domperidone base, in an amount of from 2 mg to 120 mg, domperidone maleate, in an amount equivalent to from 2 mg to 120 mg of domperidone base, and domperidone succinate (1:1), in an amount equivalent to from 2 mg to 120 mg of domperidone base; and said statin is selected from the group consisting of lovastatin, in an amount of from 2.5 mg to 80 mg or from 5 mg to 80 mg; and rosuvastatin and pharmaceutically acceptable salts and solvates thereof, in an amount per unit form equivalent to from 2.5 mg to 40 mg of rosuvastatin calcium.

Preferably, in said Component (ac), said domperidone is domperidone base, in an amount per unit form of from 2 mg to 120 mg and said statin is lovastatin, in an amount per unit form of from 5 mg to 60 mg.

The advantage of this Component (ac) of a domperidone/6-propylamino-4,5,6,7-tetrahydro-1,3-benzothiazole-2-amine/statin combination is the possibility of assuring a flexibility in the dosage and in the mode of administration of the 6-propylamino-4,5,6,7-tetrahydro-1,3-benzothiazole-2-amine, in particular of pramipexole.

pharmaceutical composition Component (ac) is administered to a patient suffering from a PMND in combination with a 6-propylamino-4,5,6,7-tetrahydro-1,3-benzothiazole-2-amine Component (b), also in a pharmaceutical composition comprising said 6-propylamino-4,5,6,7-tetrahydro-1,3-benzothiazole-2-amine in an amount equivalent to from 0.125 mg to 3000 mg of pramipexole dihydrochloride monohydrate, inclusive of a (S)-enantiomer amount equivalent to from 0.125 mg to 45 mg, normally from 15 mg to 25 mg or from 20.25 mg to 25 mg of pramipexole dihydrochloride monohydrate; and administered at a daily dose equivalent to from 0.375 mg to 3000 mg of pramipexole dihydrochloride monohydrate, inclusive of a (S)-enantiomer amount equivalent to from 0.375 mg to 45 mg, normally from 15 mg to 25 mg or from 20.25 mg to 25 mg of pramipexole dihydrochloride monohydrate. Said pharmaceutical composition Component (ab) preferably is in dosage unit form.

The above Component (ac), administered to a patient suffering from a PMND, allows for the administration to said patient of 6-propylamino-4,5,6,7-tetrahydro-1,3-benzothiazole-2-amine Component (b) daily doses inclusive a (5)-enantiomer daily dose, comprising pediatric doses and doses used in the titration period, equivalent to from 0.375 mg to 45 mg of pramipexole dihydrochloride monohydrate.

In particular, the pharmaceutical composition Component (ac) is administered to a patient suffering from a PMND in combination with a 6-propylamino-4,5,6,7-tetrahydro-1,3-benzothiazole-2-amine Component (b), also in a pharmaceutical composition comprising said 6-propylamino-4,5,6,7-tetrahydro-1,3-benzothiazole-2-amine in an amount equivalent to from 0.125 mg to 3000 mg of pramipexole dihydrochloride monohydrate, inclusive of a (S)-enantiomer amount equivalent to from 0.125 mg to 45 mg, normally from 7.5 mg to 25 mg, from 15 mg to 25 mg or from more than 20 mg to 25 mg of pramipexole dihydrochloride monohydrate; and administered at a daily dose equivalent to from 0.375 mg to 3000 mg of pramipexole dihydrochloride monohydrate, inclusive of a (S)-enantiomer amount equivalent to from 0.375 mg to 45 mg, normally from 15 mg to 25 mg, from more than 20 mg to 25 mg or from 20.25 mg to 25 mg of pramipexole dihydrochloride monohydrate. Said pharmaceutical composition Component (ac) preferably is in dosage unit form.

Preferably, said 6-propylamino-4,5,6,7-tetrahydro-1,3-benzothiazole-2-amine is selected from the group consisting of pramipexole and pharmaceutically acceptable salt and solvates thereof, also in a pharmaceutical composition in dosage unit form comprising said pramipexole or pharmaceutically acceptable salt and solvates thereof in an amount per unit form equivalent to from 0.125 mg to 45 mg, normally from 7.5 mg to 25 mg, from 15 mg to 25 mg or from more than 20 mg to 25 mg of pramipexole dihydrochloride monohydrate; and is administered to said patient at a daily dose equivalent to from 0.375 mg to 45 mg, normally from 15 mg to 25 mg or from more than 20 mg to 25 mg of pramipexole dihydrochloride monohydrate.

An advantageous Component (ab), to be used in combination with a statin Component (c) comprises or consists of a pharmaceutical composition in dosage unit form comprising
(a) domperidone or a pharmaceutically acceptable salt or solvate thereof, in an amount per unit form (in domperidone base) of from 2 mg to 120 mg; and
(b) pramipexole or a pharmaceutically acceptable salt or solvate thereof, in an amount per unit form equivalent to from 14.5 mg to 45 mg or from more than 20 mg to 45 mg, normally from 7.5 mg to 25 mg, from 15 mg to 25 mg or from 20.25 mg to 25 mg,
   in admixture with a pharmaceutical carrier or vehicle.

An advantageous Component (bc), to be used in combination with the domperidone Component (a) in the treatment of a patient suffering from a PMND, comprises or consists of a pharmaceutical composition in dosage unit form comprising pramipexole or a pharmaceutically acceptable salt or solvate thereof Component (b), in an amount per unit form equivalent to from 14.5 mg to 45 mg or from more than 20 mg to 45 mg, normally from 7.5 mg to 25 mg, from 15 mg to 25 mg or from 20.25 mg to 25 mg; and at least one synergistic agent Component (c) selected from the group consisting of fluoxetine, in an amount per unit form (in fluoxetine base, of from 2 mg to 90 mg, zonisamide or a pharmaceutically acceptable salt or solvate thereof, in an amount per unit form (in zonisamide free acid) of from 25 mg to 600 mg and a statin, in an amount per unit form of from 0.5 mg to 80 mg, in admixture with a pharmaceutical carrier or vehicle.

In the above pharmaceutical compositions (b), (ab), and (bc) of the above domperidone/6-propylamino-4,5,6,7-tetrahydro-1,3-benzothiazole-2-amine/fluoxetine, zonisamide or statin combination, including fixed-dose combinations, the 6-propylamino-4,5,6,7-tetrahydro-1,3-benzothiazole-2-amine Component (b) is pramipexole or a pharmaceutically acceptable salt or solvate thereof, in an amount per unit form of from 0.125 mg to 45 mg, in IR- or ER-formulation, to be administered at a daily dose of from 0.375 mg to 45 mg, in particular in an amount-range (in pramipexole dihydrochloride monohydrate) of from 14.5 mg to 45 mg or from more than 20 mg to 45 mg. Sub-ranges of the above pramipexole amounts per unit form and daily doses are described in “The 6-propylamino-4,5,6,7-tetrahydro-1,3-benzothiazole-2-amine Component (b)” section and are also the object of this section. Normally, said pramipexole amount-range in said compositions is equivalent to from 7.5 mg to 25 mg, from 15 mg to 25 mg or from 20.25 mg to 25 mg of pramipexole dihydrochloride monohydrate.

In particular, in said composition (including fixed-dose combinations), said domperidone is selected from the group consisting of domperidone base, domperidone hydrochloride, domperidone maleate and domperidone succinate (1:1), in an amount per unit form equivalent to from 2 mg to 120 mg of domperidone base and is administered in said composition (including fixed-dose combinations) at a daily dose equivalent to from 4 mg to 120 mg of (in domperidone base.

Specific amounts per unit form and daily doses of the above domperidone, 6-propylamino-4,5,6,7-tetrahydro-1,3-benzothiazole-2-amine, and and fluoxetine, zonisamide, or statin are described above in “The domperidone Component (a)” section, “The 6-propylamino-4,5,6,7-tetrahydro-1,3-benzothiazole-2-amine Component (b)” section, and “The synegistic agent Component (c)” section; and below, in the “Specific embodiments” section below.

As set forth above, in the treatment of a PMND, the domperidone Component (a), the 6-propylamino-4,5,6,7-tetrahydro-1,3-benzothiazole-2-amine Component (b), and the at least one synergistic agent Component (c) are used in combination each other, and the three active components may be co-administered simultaneously or sequentially, or in a fixed dose combination including a pharmaceutical composition comprising said domperidone, said 6-propylamino-4,5,6,7-tetrahydro-1,3-benzothiazole-2-amine, and said fluoxetine, zonisamide, or statin, in admixture with a pharmaceutically acceptable carrier or vehicle.

The domperidone Component (a), the 6-propylamino-4,5,6,7-tetrahydro-1,3-benzothiazole-2-amine Component (b) and the synegistic agent Component (c) can be administered separately or together in any conventional oral or parenteral dosage unit form such as capsule, tablet, powder, cachet, suspension, solution, or transdermal drug delivery system.

In the case of separate (concurrent or sequential) administration, domperidone Component (a), in an effective amount per unit form, 6-propylamino-4,5,6,7-tetrahydro-1,3-benzothiazole-2-amine Component (b), in an effective amount per unit form, and at least one synergistic agent Component (c) selected from the group consisting of fluoxetine, zonisamide and statins, in an effective amount per unit form, can each be packaged in a kit comprising said domperidone, in admixture with a pharmaceutical carrier or vehicle, said 6-propylamino-4,5,6,7-tetrahydro-1,3-benzothiazole-2-amine, preferably pramipexole, in admixture with a pharmaceutical carrier or vehicle, and said synergistic agent, in admixture with a pharmaceutical carrier or vehicle, in three separate containers.

Advantageously, said domperidone Component (a) and said synergistic agent Component (c) selected from the group consisting of fluoxetine, zonisamide and statins, in a fixed-dose combination (ac) in admixture with a pharmaceutical carrier or vehicle, may be packaged in a container; and said 6-propylamino-4,5,6,7-tetrahydro-1,3-benzothiazole-2-amine, preferably pramipexole, in admixture with a pharmaceutical carrier or vehicle, in another, separate container.

Alternatively, for example, said domperidone in admixture with a pharmaceutical carrier or vehicle, may be packaged in a container; and said 6-propylamino-4,5,6,7-tetrahydro-1,3-benzothiazole-2-amine, preferably pramipexole, and a statin, in a fixed-dose combination (bc) in admixture with a pharmaceutical carrier or vehicle, in another, separate container.

For their concurrent administration for the treatment of a PMND, said domperidone or pharmaceutically acceptable salt or solvates thereof Component (a), said 6-propylamino-4,5,6,7-tetrahydro-1,3-benzothiazole-2-amine or pharmaceutically acceptable salt or solvate thereof Component (b), and at least one synergistic agent Component (c) selected from the group consisting of fluoxetine, zonisamide and statins may also be in a fixed-dose combination (abc), formulated together in a pharmaceutical composition comprising said domperidone said 6-propylamino-4,5,6,7-tetrahydro-1,3-benzothiazole-2-amine, and said fluoxetine, zonisamide or statin, in admixture with a pharmaceutical carrier or vehicle.

The fixed-dose combinations (ac) and (abc) assure the safe, concurrent administration of domperidone and of the synergistic agent such as a statin, or of the domperidone, of the 6-propylamino-4,5,6,7-tetrahydro-1,3-benzothiazole-2-amine and of the synergistic agent, such as a statin.

### Third Aspect of the Invention

A third aspect of the present invention provides
a pharmaceutical fixed-dose combination comprising a domperidone Component (a), a 6-propylamino-4,5,6,7-tetrahydro-1,3-benzothiazole-2-amine Component (b), at least one synergistic agent Component (c) selected from the group consisting of fluoxetine, zonisamide and statins, and a pharmaceutical carrier or vehicle; and
the use of domperidone for the preparation of a medicament for the treatment of a PMND in a patient in need of said treatment, said medicament comprising or consisting of a pharmaceutical composition comprising, as an active ingredient domperidone; as a second active ingredient ingredient 6 propylamino-4,5,6,7-tetrahydro-1,3-benzothiazole-2- and, as a third active ingredient, at least one synergistic agent selected from the group consisting of fluoxetine, zonisamide and statins, in admixture with a pharmaceutical carrier or vehicle.
domperidone for use for the treatment of a PMND in a patient in need of said treatment, in a fixed-dose combination comprising an effective dose of 6-propylamino-4,5,6,7-tetrahydro-1,3-benzothiazole-2-amine, preferably pramipexole, and an effective dose of at least one synergistic agent selected from the group consisting of fluoxetine, zonisamide, and statins;
a pharmaceutical composition for use in treatment of a PMND in a patient in need of said treatment, comprising a pharmaceutically acceptable carrier or vehicle and a fixed dose combination of domperidone, a 6-propylamino-4,5,6,7-tetrahydro-1,3-benzothiazole-2-amine; and at least one synergistic agent selected from the group consisting of fluoxetine, zonisamide and statins; and
a pharmaceutical composition, normally in a dosage unit form, comprising domperidone or a pharmaceutically acceptable salt or solvate thereof, 6-propylamino-4,5,6,7-tetrahydro-1,3-benzothiazole-2-amine or a pharmaceutically acceptable salt or solvate thereof and at least one synergistic agent selected from the group consisting of fluoxetine, zonisamide and statins.

In this section, the expressions “use of domperidone for the preparation of a medicament” and “domperidone for use” are merged in the expression “use of domperidone for the preparation of a medicament (or domperidone for use)”.

Domperidone, as described in “The domperidone Component (a)” section is present as Component (a) of said fixed-dose combination or said pharmaceutical composition in an amount per unit form as described in said section, in a fixed dose combination with said 6-propylamino-4,5,6,7-tetrahydro-1,3-benzothiazole-2-amine, preferably pramipexole, Component (b), in an amount per unit form as described above in “The 6-propylamino-4,5,6,7-tetrahydro-1,3-benzothiazole-2-amine Component (b)” section, and with at least one synergistic agent-Component (c) in an amount per unit form as described above in “The synergistic agent Component (c)” section.

Said 6-propylamino-4,5,6,7-tetrahydro-1,3-benzothiazole-2-amine preferably is pramipexole or a pharmaceutically acceptable salt or solvate thereof.

According to a first embodiment, the invention provides a fixed dose combination of domperidone with 6-propylamino-4,5,6,7-tetrahydro-1,3-benzothiazole-2-amine and at least one synergistic agent selected from the group consisting of fluoxetine, zonisamide and statins useful or for use in the treatment of a PMND. Specifically this fixed-dose combination comprises or consists of
domperidone, in an amount equivalent to from 2 mg to 120 mg of domperidone base;
6-propylamino-4,5,6,7-tetrahydro-1,3-benzothiazole-2-amine, in an amount equivalent to from 0.125 mg to 3000 mg of pramipexole dihydrochloride monohydrate, including a (S)-enantiomer amount equivalent to from 0.125 mg to 45 mg of pramipexole dihydrochloride monohydrate; and
at least one synergistic agent selected from the group consisting of fluoxetine, in an amount equivalent to from 2 mg to 90 mg of fluoxetine base, zonisamide, in an amount equivalent to from 25 mg to 600 mg of zonisamide free acid; and a statin, in an amount of from 0.5 mg to 80 mg.

According to a second embodiment, the invention provides the use of domperidone for the preparation of a medicament (or domperidone for use) for the treatment of PMND, and the medicament itself, said medicament being a pharmaceutical composition in dosage unit form comprising
(a) domperidone or a pharmaceutically acceptable salt or solvate thereof, in an amount per unit form equivalent to from 2 mg to 120 mg of domperidone base;
(b) 6-propylamino-4,5,6,7-tetrahydro-1,3-benzothiazole-2-amine or a pharmaceutically acceptable salt or solvate thereof, in an amount per unit form equivalent to from 0.125 mg to 3000 mg of pramipexole dihydrochloride monohydrate, including a (S)-enantiomer amount per unit form equivalent to form 0.125 mg to 45 mg of pramipexole dihydrochloride monohydrate; and
(c) at least one synergistic agent selected from the group consisting of fluoxetine, in an amount per unit form of from 2 mg to 90 mg, zonisamide, in an amount per unit form of from 25 mg to 600 mg, and a statin, in an amount per unit form of from 0.5 mg to 80 mg,
   in admixture with a pharmaceutical carrier or vehicle.

According to a third embodiment, the invention provides a pharmaceutical composition for use for the treatment of a PMND in a patient comprising a pharmaceutical carrier or vehicle and a fixed-dose combination of domperidone Component (a), 6-propylamino-4,5,6,7-tetrahydro-1,3-benzothiazole-2-amine Component (b) and at least one synergistic agent Component (c) selected from the group consisting of fluoxetine, zonisamide and statins.

According to this third embodiment, in said composition, domperidone is present in an amount equivalent to from 2 mg to 120 mg of domperidone base; 6-propylamino-4,5,6,7-tetrahydro-1,3-benzothiazole-2-amine is present in an amount equivalent to from 0.125 mg to 3000 mg of pramipexole dihydrochloride monohydrate, including a (S)-enantiomer amount per unit form equivalent to form 0.125 mg to 45 mg of pramipexole dihydrochloride monohydrate; and the synergistic agent is selected from the group consisting of fluoxetine, in an amount of from 2 mg to 90 mg, zonisamide, in an amount of from 25 mg to 600 mg, and a statin, in an amount of from 0.5 mg to 80 mg.

According to a fourth embodiment of this third aspect, the invention also provides a pharmaceutical composition comprising
(a) domperidone or a pharmaceutically acceptable salt or solvate thereof;
(b) 6-propylamino-4,5,6,7-tetrahydro-1,3-benzothiazole-2-amine or a pharmaceutically acceptable salt or solvate thereof; and
(c) at least one synergistic agent selected from the group consisting of fluoxetine and pharmaceutically acceptable salts and solvates thereof, zonisamide and pharmaceutically acceptable salts and solvates thereof, and statins;
   in admixture with a pharmaceutical carrier or vehicle.

The above pharmaceutical compositions comprising said domperidone or pharmaceutically acceptable salt or solvate thereof Component (a), said 6-propylamino-4,5,6,7-tetrahydro-1,3-benzothiazole-2-amine or pharmaceutically acceptable salt or solvate thereof Component (b) and said synergistic agent Component (c) normally are in a dosage unit form and said amount of said domperidone, of said 6-propylamino-4,5,6,7-tetrahydro-1,3-benzothiazole-2-amine and, respectively, of said synergistic agent is per unit form.

According to this fourth embodiment, the invention provides a pharmaceutical composition in dosage unit form comprising
(a) domperidone or a pharmaceutically acceptable salt or solvate thereof, in an amount per unit form equivalent to from 2 mg to 120 mg of domperidone base;
(b) 6-propylamino-4,5,6,7-tetrahydro-1,3-benzothiazole-2-amine or a pharmaceutically acceptable salt or solvate thereof, in an amount per unit form equivalent to from 0.125 mg to 3000 mg of pramipexole dihydrochloride monohydrate, including a (S)-enantiomer amount per unit form equivalent to form 0.125 mg to 45 mg of pramipexole dihydrochloride monohydrate; and
(c) at least one synergistic agent selected from the group consisting of fluoxetine, in an amount per unit form of from 2 mg to 90 mg, zonisamide, in an amount per unit form of from 25 mg to 600 mg, and a statin, in an amount per unit form of from 0.5 mg to 80 mg,
   in admixture with a pharmaceutical carrier or vehicle.

In particular, in this pharmaceutical composition, said 6-propylamino-4,5,6,7-tetrahydro-1,3-benzothiazole-2-amine is selected from the group consisting of the racemate or a pharmaceutically acceptable salt thereof, in an amount per unit form equivalent to from 0.25 mg to 90 mg, normally from 30 mg to 50 mg or from more than 20 mg to 50 mg of pramipexole dihydrochloride monohydrate, pramipexole or a pharmaceutically acceptable salt thereof, in an amount per unit form equivalent to from 0.125 mg to 45 mg, normally from 7.5 mg, from 15 mg to 25 mg or from more than 20 mg to 25 mg of pramipexole dihydrochloride monohydrate, and a (R)/(S)-mixture, in an amount per unit form equivalent to from 50 mg to 3000 mg of pramipexole dihydrochloride monohydrate , inclusive of a (S)-enantiomer amount per unit form equivalent to from 0.125 mg to 45 mg, normally from 7.5 mg to 25 mg, from 15 mg to 25 mg or from more than 20 mg to 25 mg of pramipexole dihydrochloride monohydrate.

More particularly, according to this fourth above embodiment, the invention provides a pharmaceutical composition in dosage unit form comprising
(a) domperidone or a pharmaceutically acceptable salt or solvate thereof, in an amount per unit form equivalent to from 2 mg to 120 mg of domperidone base;
(b) a 6-propylamino-4,5,6,7-tetrahydro-1,3-benzothiazole-2-amine selected from the group consisting of pramipexole and pharmaceutically acceptable salts and solvates thereof, in an amount per unit form equivalent to from more than 20 mg to 45 mg, normally from more than 20 mg to 25 mg or from 20.25 mg to 25 mg of pramipexole dihydrochloride monohydrate; and
(c) at least one synergistic agent selected from the group consisting of fluoxetine and pharmaceutically acceptable salts and solvates thereof, in an amount per unit form equivalent to from 2 mg to 90 mg of fluoxetine base, zonisamide, in an amount per unit form equivalent to from 25 mg to 600 mg of zonisamide free acid, and a statin, in an amount per unit form of from 0.5 mg to 80 mg;
   in admixture with a pharmaceutical carrier or vehicle.

According to the above embodiments of this third aspect, the invention provides a pharmaceutical composition comprising a pharmaceutical carrier or vehicle and a fixed-dose combination of
(a) domperidone, in an amount equivalent to from 2 mg to 120 mg of domperidone base;
(b) a 6-propylamino-4,5,6,7-tetrahydro-1,3-benzothiazole-2-amine, in an amount equivalent to from 0.125 mg to 3000 mg of pramipexole dihydrochloride monohydrate, including a (S)-enantiomer amount equivalent to form 0.125 mg to 45 mg, normally from 7.5 mg to 25 mg, from 15 mg to 25 mg or from more than 20 mg to 25 mg of pramipexole dihydrochloride monohydrate; and
(c) at least one synergistic agent selected from the group consisting of fluoxetine, in an amount (in fluoxetine base) of from 2 mg to 90 mg, zonisamide, in an amount per unit form (in zonisamide free acid) of from 25 mg to 600 mg, and a statin, in an amount of from 0.5 mg to 80 mg.

This pharmaceutical composition is useful or for use for the treatment of a PMND in a patient in need of said treatment.

Preferably, in the above use and compositions of the above embodiments, said fluoxetine or pharmaceutically acceptable salt or solvate thereof is fluoxetine base or fluoxetine hydrochloride.

Preferably, in the above use and compositions, said zonisamide or pharmaceutically acceptable salt or solvate thereof is zonisamide free acid.

Preferably, in the above use and compositions, said statin is selected from the group consisting of atorvastatin and pharmaceutically acceptable salts and solvates thereof, fluvastatin and pharmaceutically acceptable salts and solvates thereof, lovastatin, pitavastatin and pharmaceutically acceptable salts and solvates thereof, pravastatin and pharmaceutically acceptable salts and solvates thereof, rosuvastatin and pharmaceutically acceptable salts and solvates thereof; and simvastatin.

According to an advantageous embodiment of this third aspect, the invention provides a pharmaceutical composition in dosage unit form comprising a pharmaceutical carrier or vehicle, domperidone Component (a), a 6-propylamino-4,5,6,7-tetrahydro-1,3-benzothiazole-2-amine Component (b) and a statin Component (c), wherein

said domperidone is present in an amount per unit form equivalent to from 2 mg to 120 mg of domperidone base;

said 6-propylamino-4,5,6,7-tetrahydro-1,3-benzothiazole-2-amine is selected from the group consisting of the racemate or a pharmaceutically acceptable salt thereof, in an amount per unit form equivalent to from 0.25 mg to 90 mg, normally from 30 mg to 50 mg or from more than 20 mg to 50 mg of pramipexole dihydrochloride monohydrate, pramipexole or a pharmaceutically acceptable salt thereof, in an amount per unit form equivalent to from 0.125 mg to 45 mg, normally from 15 mg to 25 mg or from more than 20 mg to 25 mg of pramipexole dihydrochloride monohydrate, and a (R)/(S)-mixture, in an amount per unit form equivalent to from 50 mg to 3000 mg of pramipexole dihydrochloride monohydrate , inclusive of a (S)-enantiomer amount per unit form equivalent to from 0.125 mg to 45 mg, normally from 15 mg to 25 mg or from more than 20 mg to 25 mg of pramipexole dihydrochloride monohydrate; and

said statin is selected from the group consisting of atorvastatine and pharmaceutically acceptable salts and solvates thereof, in an amount per unit form equivalent to from 5 mg to 80 mg, normally from 5 mg to 60 mg of atorvastatin free acid; fluvastatin and pharmaceutically acceptable salts and solvates thereof, in an amount per unit form equivalent to from 10 mg to 80 mg, normally from 10 mg to 60 mg of fluvastatin free acid; lovastatin, in an amount of from 2.5 mg to 80 mg or from 5 mg to 80 mg, normally from 5 mg to 60 mg; pitavastatin and pharmaceutically acceptable salts and solvates thereof, in an amount per unit form equivalent to from 0.5 mg to 4 mg, normally from 0.5 mg to 3 mg of pitavastatin free acid; pravastatin and pharmaceutically acceptable salts and solvates thereof, in an amount per unit form equivalent to from 2.5 mg to 60 mg, normally from 2.5 mg to 40 mg of pravastatin sodium; rosuvastatin and pharmaceutically acceptable salts and solvates thereof, in an amount per unit form equivalent to from 2.5 mg to 40 mg, normally from 2.5 mg to 30 mg of rosuvastatin calcium; and simvastatin, in an amount per unit form of from 2.5 mg to 40 mg, normally from 2.5 mg to 30 mg.

According to the above advantageous embodiment of this third aspect, in this composition said domperidone Component (a) is selected from the group consisting of domperidone base, domperidone hydrochloride, domperidone maleate and domperidone succinate (1:1), in an amount per unit form (in domperidone base) of from 2 mg to 120 mg; said 6-propylamino-4,5,6,7-tetrahydro-1,3-benzothiazole-2-amine Component (b) is selected from the group consisting of pramipexole and pharmaceutically acceptable salts and solvates thereof, in an amount equivalent to from 0.125 mg to 45 mg, normally from 7.5 mg (or more than 7.5 mg) to 25 mg, from 15 mg to 25 mg, from more than 20 mg to 25 mg, or from 20.25 mg to 25 mg of pramipexole dihydrochloride monohydrate; and said statin Component (c) is selected from the group consisting of rosuvastatin and pharmaceutically acceptable salts and solvates thereof, in an amount equivalent to from 2.5 mg to 40 mg of rosuvastatin calcium, and lovastatin, in an amount of from 2.5 mg to 80 mg or from 5 mg to 80 mg.

A specific, preferred fixed-dose combination (abc) according to the above embodiments of this third aspect is in a pharmaceutical composition comprising from 2 mg to 120 mg of domperidone base; pramipexole or a pharmaceutically acceptable salt or solvate thereof, in an amount equivalent to from 0.125 mg to 45 mg, normally from 7.5 mg (or more than 7.5 mg) to 25 mg, from 15 mg to 25 mg, from more than 20 mg to 25 mg or from 20.25 mg to 25 mg of pramipexole dihydrochloride monohydrate; and from 5 mg to 60 mg or from 10 mg to 60 mg of lovastatin.

In the compositions of the above embodiments the 6-propylamino-4,5,6,7-tetrahydro-1,3-benzothiazole-2-amine is advantageously selected from the group consisting of the racemate or a pharmaceutically acceptable salt thereof, in an amount per unit form equivalent to from 0.25 mg to 90 mg, normally from 15 mg to 25 mg, from 30 mg to 50 mg or from more than 40 mg to 50 mg of pramipexole dihydrochloride monohydrate; pramipexole or a pharmaceutically acceptable salt thereof, in an amount equivalent to from 0.125 mg to 45 mg, normally from 7.5 mg to 25 mg, from 15 mg to 25 mg or from more than 20 mg to 25 mg of pramipexole dihydrochloride monohydrate; and a (R)/(S)-mixture, in an amount per unit form equivalent to from 50 mg to 3000 mg of pramipexole dihydrochloride monohydrate, inclusive of a (S)-enantiomer amount per unit form equivalent to from 0.125 mg to 45 mg, normally from 7.5 mg to 25 mg, from 15 mg to 25 mg or from more than 20 mg to 25 mg of pramipexole dihydrochloride monohydrate.

According to the above embodiments, in said pharmaceutical composition, useful or for use for the treatment of a PMND, said domperidone may be present in an amount per unit form may be equivalent to a range selected from the group consisting of from 2 mg to 120 mg, 2 mg to 100 mg, from 2 mg to 80 mg, from 2 mg to 60 mg, from 2 mg to 40 mg, from 2 mg to 30 mg and from 2 mg to 20 mg, normally from 10 mg to 120 mg, from 10 mg to 100 mg, from 10 mg to 80 mg, from 10 mg to 60 mg, from 10 mg to 40 mg, from 10 mg to 30 mg and from 10 mg to 20 mg of domperidone base. Preferably said domperidone Component (a) is present as domperidone base, domperidone hydrochloride, domperidone maleate or domperidone succinate (1:1).

Said compositions (abc) of this advantageous embodiment of the this third aspect of the invention normally are in dosage unit form and the above amounts of domperidone Component (a), pramipexole Component (b) and statin Component (c) are per unit form.

According to the above embodiments, in said pharmaceutical composition, useful or for use for the treatment of a PMND, said pramipexole may be present in an amount per unit form of from more than 4.5 mg to 45 mg, from more than 6 mg to 45 mg and even from 6.5 mg to 45 mg, from 7.5 mg (or more than 7.5 mg) to 25 mg, from 15 mg to 25 mg, from more than 20 mg to 25 mg or from 20.25 mg to 25 mg of pramipexole dihydrochloride monohydrate of pramipexole dihydrochloride monohydrate.

Specific amounts per unit form of the domperidone Component (a), of the 6-propylamino-4,5,6,7-tetrahydro-1,3-benzothiazole-2-amine Component (b), and of the statin Component (c) active ingredients, in particular the amounts per unit form sub-ranges of said Component (a), of said Component (b) and, respectively, of said Component (c), are described above in “The domperidone Component (a)”, and in “The 6-propylamino-4,5,6,7-tetrahydro-1,3-benzothiazole-2-amine Component (b)”, and, respectively, in “The synergistic agent Component (c)” sections.

### Specific Embodiments

As mentioned above, for the treatment of a PMND, the domperidone Component (a), the 6-propylamino-4,5,6,7-tetrahydro-1,3-benzothiazole-2-amine Component (b) and the synergistic agent Component (c) are formulated, separately or in fixed-dose combinations, in a pharmaceutical compositions in dosage unit form, each in admixture with a pharmaceutical carrier or vehicle.

Thus, according to the three aspects of the invention, for the method (or use) described above each of Component (a), Component (b), Component (c), fixed-dose combination (ab), fixed-dose combination (ac), fixed-dose combination (bc) and fixed-dose combination (abc) is formulated in pharmaceutical compositions comprising an effective amount of domperidone, an effective amount of 6 propylamino-4,5,6,7-tetrahydro-1,3-benzothiazole-2-amine and an an effective amount of at least one synergistic agent selected from the group consisting of fluoxetine, or zonisamide or a statin, in admixture with a pharmaceutical carrier or vehicle.

Normally, said Component (a), Component (b), Component (c), fixed-dose combination (ab), fixed-dose combination (ac), fixed-dose combination (bc) and fixed-dose combination (abc) pharmaceutical compositions are formulated in dosage unit form in admixture with a pharmaceutical carrier or vehicle, herein below referred to as “Unit Form”. In the Unit Form, each of the Component (a), Component (b) and Component (c) may also be formulated separately, each in admixture with a pharmaceutical carrier or vehicle.

As also set forth above, for said use in the combination of the present invention, domperidone is present in a Unit Form in an amount per unit form equivalent to from 2 mg to 120 mg of domperidone base, and is administered at a daily dose equivalent to from 4 mg to 120 mg of domperidone base.

In the Unit Form, the domperidone Component (a) active ingredient is advantageously selected from the group consisting of domperidone free base, domperidone hydrochloride, domperidone maleate and domperidone succinate (1:1), in an amount equivalent to from 2 mg to 120 mg of domperidone base, and is in admixture with a pharmaceutical carrier or vehicle.

The amount of domperidone Component (a) in an IR-formulated Unit Form is normally equivalent to from 2 mg to 60 mg of domperidone base, in particular equivalent to from 2 mg to 50 mg, from 2 mg to 40 mg, from 2 mg to 30 mg, from 2 mg to 25 mg, from 2 mg to 20 mg, from 2 mg to 15 mg or from 2 mg to 10 mg of domperidone base, advantageously from 10 mg to 60 mg, from 10 mg to 40 mg, from 10 mg to 30 mg, from 10 mg to 25 mg, from 10 mg to 20 mg, or from 10 mg to 15 mg of domperidone base.

The amount of domperidone Component (a) in an ER-formulated Unit Form is normally equivalent equivalent to from 4 mg to 120 mg, in particular equivalent to from 4 mg to 100 mg, from 4 mg to 80 mg, from 4 mg to 60 mg, from 4 to 50 mg, from 4 mg to 40 mg, from 4 mg to 30 mg or from 4 to 20 mg of domperidone base, normally equivalent to from 10 mg to 120 mg, from 10 to 100 mg, from 10 to 80 mg, from 10 mg to 60 mg, from 10 to 50 mg, from 10 mg to 40 mg, from 10 to 30 mg or from 10 mg to 20 mg of domperidone base.

In the (or for its use in) combination with domperidone and with fluoxetine, zonisamide or a statin, the 6-propylamino-4,5,6,7-tetrahydro-1,3-benzothiazole-2-amine Component (b) is present in the Unit Form in an amount equivalent to from 0.125 mg to 3000 mg of pramipexole dihydrochloride monohydrate, including a (S) enantiomer amount equivalent to from 0.125 mg to 45 mg of pramipexole dihydrochloride monohydrate.

In an IR-formulated Unit Form, the amount of the 6-propylamino-4,5,6,7-tetrahydro-1,3-benzothiazole-2-amine Component (b) is normally equivalent to from 0.125 mg to 1500 mg, advantageously from 1.6 mg to 1500 mg preferably from 1.625 mg to 1500 mg of pramipexole dihydrochloride monohydrate, including a (S)-enantiomer amount equivalent to from 0.125 mg to 22.5 mg, advantageously from 1.6 mg to 22.5 mg preferably from 1.625 mg to 22.5 mg, normally from 7.5 mg to 12.5 mg or from more than 10 mg to 12.5 mg of pramipexole dihydrochloride monohydrate, depending on safety and tolerability, in combination with the domperidone Component (a) and with at least one of fluoxetine, zonisamide, or a statin Component (c).

In an ER-formulated Unit Form, the amount of 6-propylamino-4,5,6,7-tetrahydro-1,3-benzothiazole-2-amine Component (b) will normally be in a range equivalent to from 0.375 mg to 3000 mg, normally from 1.5 mg to 3000 mg of pramipexole dihydrochloride monohydrate, including a (S)-enantiomer amount equivalent to from 0.375 mg to 45 mg, advantageously from more than 4.5 mg to 45 mg, preferably from more than 6 mg to 45 mg, from 14.5 mg to 45 mg, normally from 15 mg to 25 mg or from more than 20 mg to 25 mg of pramipexole dihydrochloride monohydrate, depending on safety and tolerability, in combination with the domperidone Component (a) and with at least one of fluoxetine, zonisamide, or a statin Component (c).

In the Unit Form, the 6-propylamino-4,5,6,7-tetrahydro-1,3-benzothiazole-2-amine Component (b) active ingredient is selected from the group consisting of
(S)-6-propylamino-4,5,6,7-tetrahydro-1,3-benzothiazole-2-amine (pramipexole) and pharmaceutically acceptable salts and solvates thereof, in an amount equivalent to from 0.125 mg to 45 mg, normally from 15 mg to 25 mg or from more than 20 mg to 25 mg of pramipexole dihydrochloride monohydrate;
(R,S)-6-propylamino-4,5,6,7-tetrahydro-1,3-benzothiazole-2-amine (the racemate) and pharmaceutically acceptable salts an solvates thereof, in an amount equivalent to from 0.25 mg to 90 mg, normally from 30 mg to 50 mg or from more than 40 mg to 50 mg of pramipexole dihydrochloride monohydrate; and
a (R)/(S)-mixture comprising 6-propylamino-4,5,6,7-tetrahydro-1,3-benzothiazole-2-amine, in an amount equivalent to from 50 mg to 3000 mg of pramipexole dihydrochloride monohydrate, including a (S)-enantiomer amount equivalent to from 0.125 mg to 45 mg, normally from 15 mg to 25 mg or from more than 20 mg to 25 mg of pramipexole dihydrochloride monohydrate.

A typical Unit Form (abc) comprises a pharmaceutical composition comprising
(a) domperidone in an amount equivalent to from 2 mg to 120 mg of domperidone base;
(b) 6-propylamino-4,5,6,7-tetrahydro-1,3-benzothiazole-2-amine selected from the group consisting of the racemate or a pharmaceutically acceptable salt thereof, in an amount equivalent to from 0.25 mg to 90 mg, normally from 30 mg to 50 mg or from more than 40 mg to 50 mg of pramipexole dihydrochloride monohydrate, pramipexole or a pharmaceutically acceptable salt thereof, in an amount equivalent to from 0.125 mg to 45 mg, normally from 15 mg to 25 mg or from more than 20 mg to 25 mg of pramipexole dihydrochloride monohydrate, and a (R)/(S)-mixture, in an amount equivalent to from 50 mg to 3000 mg, inclusive of a (S)-enantiomer amount equivalent to from 0.125 mg to 45 mg, normally from 15 mg to 25 mg or from more than 20 mg to 25 mg of pramipexole dihydrochloride monohydrate; and
(c) at least one synergistic agent selected from the group consisting of fluoxetine, in an amount (in fluoxetine base) of from 2 mg to 90 mg, or zonisamide, in an amount (in zonisamide free acid) of from 25 mg to 600 mg, or a statin, in an amount of from 0.5 mg to 80 mg; and
   a pharmaceutical carrier or vehicle.

Advantageously, said Unit Form (abc) comprises, as a 6-propylamino-4,5,6,7-tetrahydro-1,3-benzothiazole-2-amine active ingredient (b), pramipexole or pharmaceutically acceptable salt or solvate thereof in an amount equivalent to from 0.125 mg to 45 mg, normally from 15 mg to 25 mg or from more than 20 mg to 25 mg of pramipexole dihydrochloride monohydrate; and a pharmaceutical carrier or vehicle.

According to a specific embodiment of the three aspects of the invention, in the Unit Form said 6-propylamino-4,5,6,7-tetrahydro-1,3-benzothiazole-2-amine Component (b) is pramipexole or a pharmaceutically acceptable salt or solvate thereof, in an amount as illustrated in the “The 6-propylamino-4,5,6,7-tetrahydro-1,3-benzothiazole-2-amine Component (b)” section, in admixture with a pharmaceutical carrier or vehicle.

In the Unit Form, said 6-propylamino-4,5,6,7-tetrahydro-1,3-benzothiazole-2-amine Component (b) may be pramipexole or a pharmaceutically acceptable salt or solvate thereof, in an amount per IR-unit form equivalent to from 0.125 mg to 30 mg or from 0.125 mg to 22.5 mg, up to from 7.25 mg to 22.5 mg or from 20.25 mg to 22.5 mg of pramipexole dihydrochloride monohydrate, normally equivalent to a range selected from the group consisting of from 0.125 mg to 30 mg, from 0.125 mg to 22.5 mg, from 0.125 mg to 11.25 mg, 0.125 mg to 15 mg, and from 0.125 mg to 10 mg of pramipexole dihydrochloride monohydrate, depending on safety and tolerability, in combination with the domperidone Component (a) and with at least one synergistic agent selected from the group consisting of fluoxetine, zonisamide, or a statin Component (c).

Advantageously, said pramipexole or a pharmaceutically acceptable salt or solvate thereof, is in an amount in IR-formulation equivalent to a range selected from the group consisting of from 3 mg to 22.5 mg, from more than 4.5 mg to 22.5 mg, from 4.8 mg to 22.5 mg, from more than 6 mg to 22.5 mg, from 7.25 mg to 22.5 mg, from more than 10 mg to 22.5 mg, and from 20.25 mg to 22.5 mg of pramipexole dihydrochloride monohydrate.

Preferably, said pramipexole or pharmaceutically acceptable salt or solvate thereof active ingredient is in an amount in IR-formulation equivalent to a range selected from the group consisting of from more than 10 mg to 22.5 mg, from 14.5 mg to 22.5 mg, from 15 mg to 22.5 mg, from 17.5 mg to 22.5 mg, and from 20 mg to 22.5 mg and from 20.25 mg to 22.5 mg of pramipexole dihydrochloride monohydrate.

Normally, in this Unit Form, said pramipexole or pharmaceutically acceptable salt thereof is present in an amount per IR-unit form in an amount equivalent to from 7.5 mg or 12.5 mg or from more than 10 mg to 12.5 mg of pramipexole dihydrochloride monohydrate.

According to a particular embodiment, as described in “The formulations” section below, said 6-propylamino-4,5,6,7-tetrahydro-1,3-benzothiazole-2-amine is pramipexole or a pharmaceutically acceptable salt or solvate thereof, in an ER-formulation, in an amount equivalent to a range selected from the group consisting of from more than 4.5 mg to 45 mg, from 4.8 mg to 45 mg, from more than 6 mg to 45 mg, and from more than 10 mg to 45 mg of pramipexole dihydrochloride monohydrate.

Said pramipexole or pharmaceutically acceptable salt or solvate thereof active ingredient in ER-formulation may also be present in an amount equivalent to a range selected from the group consisting of from more than 10 mg to 45 mg, from 14.5 mg to 45 mg, from 15 mg to 45 mg, from 20.25 mg to 45 mg, from 20.25 mg to 40 mg, from 20.25 mg to 35 mg, from 20.25 mg to 30 mg and from 20.25 mg to 25 mg of pramipexole dihydrochloride monohydrate.

Preferably, said pramipexole or pharmaceutically acceptable salt or solvate thereof active ingredient in ER-formulation is present in an amount equivalent to from 15 mg to 25 mg or from more than 20 mg to 25 mg of pramipexole dihydrochloride monohydrate.

In the combination with domperidone and with 6-propylamino-4,5,6,7-tetrahydro-1,3-benzothiazole-2-amine, the at least one synergistic agent Component (c) is selected from the group consisting of
fluoxetine, which is present in the Unit Form in an amount equivalent to from 2 mg to 90 mg of fluoxetine base;
zonisamide, which is present in the Unit Form in an amount of from 25 mg to 600 mg; and
the statin, which is present in the Unit Form in an amount of from 0.5 mg to 80 mg.

When fluoxetine Component (c) is not formulated in a fixed-dose combination, the Unit Form may also be the specific 90 mg ER-weekly preparation, to be administered once a week, in combination either with Component (a) and with Component (b), or with Component (ab).

According to a specific embodiment of the three aspects of the invention, the invention provides an Unit Form consisting of the fluoxetine specific 90 mg ER-weekly preparation Component (c), for use for the treatment of a PMND in a patient in need of said treatment, in combination with a Component (ab), consisting of a pharmaceutical composition comprising a pharmaceutical carrier and a fixed-dose combination of domperidone or a pharmaceutically acceptable salt or solvate thereof and pramipexole or pharmaceutically acceptable salt or solvate thereof.

According to a specific embodiment of the three aspects of the invention, the domperidone/6-propylamino-4,5,6,7-tetrahydro-1,3-benzothiazole-2-amine/fluoxetine combination is selected from the group consisting of
a domperidone Component (a), in a pharmaceutical composition comprising an active ingredient selected from the group consisting of domperidone and pharmaceutically acceptable salts and solvates thereof, in an amount per unit form equivalent to from 2 mg to 120 mg of domperidone base, in admixture with a pharmaceutical carrier or vehicle in IR or ER formulation,
   for the treatment of a PMND in a patient in combination with Component (bc) in a pharmaceutical composition in dosage unit form comprising pramipexole or pharmaceutically acceptable salt or solvate thereof active ingredient (b), in an amount per unit form equivalent to from 0.125 mg to 22.5 mg, normally from 7.5 mg to 12.5 mg or from more than 10 mg to 12.5 mg of pramipexole dihydrochloride monohydrate; and fluoxetine or a pharmaceutically acceptable salt or solvate thereof active ingredient (c), in an amount per unit form equivalent to from 2 mg to 40 mg of fluoxetine base, in admixture with a pharmaceutical carrier or vehicle in IR-formulation;
a domperidone Component (a), in a pharmaceutical composition in dosage unit form comprising an active ingredient selected from the group consisting of domperidone and pharmaceutically acceptable salts and solvates thereof, in an amount per unit form equivalent to from 2 mg to 120 mg of domperidone base, in admixture with a pharmaceutical carrier or vehicle in IR or ER formulation,
   for the treatment of a PMND in a patient in combination with Component (bc) in a pharmaceutical composition in dosage unit form comprising pramipexole or pharmaceutically acceptable salt or solvate thereof active ingredient, in an amount per unit form equivalent to from 0.375 mg to 45 mg, normally from 15 mg to 25 mg or from more than 20 mg to 25 mg of pramipexole dihydrochloride monohydrate; and fluoxetine or a pharmaceutically acceptable salt or solvate thereof active ingredient (c), in an amount per unit form equivalent to from 4 mg to 90 mg of fluoxetine base, in admixture with a pharmaceutical carrier or vehicle in ER-formulation to be administered once a day;
a Component (ab), as a pharmaceutical composition i8n dosage unit form comprising a domperidone active ingredient (a) selected from the group consisting of domperidone and pharmaceutically acceptable salts and solvates thereof, in an amount per unit form equivalent to from 2 mg to 120 mg of domperidone base; and pramipexole or pharmaceutically acceptable salt or solvate thereof active ingredient (b), in an amount per unit form equivalent to from 0.125 mg to 45 mg, normally from 15 mg to 25 mg or from more than 20 mg to 25 mg of pramipexole dihydrochloride monohydrate in admixture with a pharmaceutical carrier or vehicle;
   for the treatment of a PMND in combination with Component (c), as a pharmaceutical composition in dosage unit form comprising fluoxetine or a pharmaceutically acceptable salt or solvate thereof active ingredient, in an amount per unit form equivalent to from 2 mg to 90 mg of fluoxetine base, in admixture with a pharmaceutical carrier or vehicle;
a Component (ac), as a pharmaceutical composition in dosage unit form comprising domperidone active ingredient (a) selected from the group consisting of domperidone and pharmaceutically acceptable salts and solvates thereof, in an amount equivalent to from 2 mg to 120 mg of domperidone base; and fluoxetine or pharmaceutically acceptable salt or solvate thereof active ingredient (c), in an amount per unit form equivalent to from 2 mg to 90 mg of fluoxetine base, in admixture with a pharmaceutical carrier or vehicle in an IR or ER formulation to be administered once or twice a day;
   for the treatment of a PMND in combination with Component (b), as a pharmaceutical composition comprising 6-propylamino-4,5,6,7-tetrahydro-1,3-benzothiazole-2-amine active ingredient, in an amount per unit form equivalent to from 0.125 mg to 3000 mg of pramipexole dihydrochloride monohydrate, including a (S)-enantiomer amount per unit form equivalent to from 0.125 mg to 45 mg, normally from 7.5 mg (or more than 7.5 mg) to 25 mg or from 15 mg to 25 mg of pramipexole dihydrochloride monohydrate, in admixture with a pharmaceutical carrier or vehicle.

According to a specific embodiment of the three aspects of the invention, the domperidone/6-propylamino-4,5,6,7-tetrahydro-1,3-benzothiazole-2-amine/zonisamide combination is selected from the group consisting of
a domperidone Component (a), in a pharmaceutical composition in dosage unit form comprising an active ingredient selected from the group consisting of domperidone and pharmaceutically acceptable salts and solvates thereof, in an amount per unit form equivalent to from 2 mg to 120 mg of domperidone base, in admixture with a pharmaceutical carrier or vehicle in IR or ER formulation,
   for the treatment of a PMND in a patient in combination with Component (bc), in a pharmaceutical composition in dosage unit form comprising pramipexole or pharmaceutically acceptable salt or solvate thereof active ingredient (b), in an amount per unit form equivalent to from 0.125 mg to 22.5 mg, normally from 7.5 mg to 12.5 mg or from more than 10 mg to 12.5 mg of pramipexole dihydrochloride monohydrate; and zonisamide active ingredient (c), in an amount per unit form of from 25 mg to 200 mg, in admixture with a pharmaceutical carrier or vehicle in IR-formulation;
a domperidone Component (a), in a pharmaceutical composition in dosage unit form comprising an active ingredient (a) selected from the group consisting of domperidone and pharmaceutically acceptable salts and solvates thereof, in an amount equivalent to from 2 mg to 120 mg of domperidone base, in admixture with a pharmaceutical carrier or vehicle in IR or ER formulation,
   for the treatment of a PMND in a patient in combination with Component (bc) in a pharmaceutical composition in dosage unit form comprising pramipexole or pharmaceutically acceptable salt or solvate thereof active ingredient (b), in an amount per unit form equivalent to from 0.375 mg to 45 mg, normally from 15 mg to 25 mg or from more than 20 mg to 25 mg of pramipexole dihydrochloride monohydrate; and zonisamide active ingredient (c), in an amount per unit form of from 25 mg to 600 mg, in admixture with a pharmaceutical carrier or vehicle in IR- or ER-formulation; and
a Component (ab), as a pharmaceutical composition in dosage unit form comprising a domperidone active ingredient (a) selected from the group consisting of domperidone and pharmaceutically acceptable salts and solvates thereof, in an amount per unit form equivalent to from 2 mg to 120 mg of domperidone base; and pramipexole or pharmaceutically acceptable salt or solvate thereof active ingredient (b), in an amount per unit form equivalent to from 0.125 mg to 45 mg, normally from 7.5 mg (or more than 7.5 mg) to 25 mg or from 15 mg to 25 mg of pramipexole dihydrochloride monohydrate, in admixture with a pharmaceutical carrier or vehicle;
   for the treatment of a PMND in combination with Component (c), as a pharmaceutical composition comprising a zonisamide active ingredient (c), in an amount per unit form of from 25 mg to 600 mg, in admixture with a pharmaceutical carrier or vehicle in an IR or ER formulation to be administered once or twice a day; and
a Component (ac), as a pharmaceutical composition in dosage unit form comprising a domperidone active ingredient (a) selected from the group consisting of domperidone and pharmaceutically acceptable salts and solvates thereof, in an amount equivalent to from 2 mg to 120 mg of domperidone base; and zonisamide active ingredient (c), in an amount per unit form of from 25 mg to 600 mg, in admixture with a pharmaceutical carrier or vehicle in an IR or ER formulation to be administered once or twice a day;
   for the treatment of a PMND in combination with Component (b), as a pharmaceutical composition comprising 6-propylamino-4,5,6,7-tetrahydro-1,3-benzothiazole-2-amine active ingredient in an amount per unit form equivalent to from 0.125 mg to 3000 mg of pramipexole dihydrochloride monohydrate, including a (S)-enantiomer amount per unit form equivalent to from 0.125 mg to 45 mg, normally from 7.5 mg (or more than 7.5 mg) to 25 mg or from 15 mg to 25 mg, of pramipexole dihydrochloride monohydrate, in admixture with a pharmaceutical carrier or vehicle.

According to specific embodiment of the three aspects of the invention, the domperidone/6-propylamino-4,5,6,7-tetrahydro-1,3-benzothiazole-2-amine/statin combination is selected from the group consisting of
a domperidone Component (a), in an Unit Form which comprises a pharmaceutical composition comprising an active ingredient selected from the group consisting of domperidone and pharmaceutically acceptable salts and solvates thereof, in an amount equivalent to from 2 mg to 120 mg of domperidone base, in admixture with a pharmaceutical carrier or vehicle in IR or ER formulation,
   for the treatment of a PMND in a patient in combination with Component (bc), in an Unit Form which comprises a pharmaceutical composition comprising pramipexole or pharmaceutically acceptable salt or solvate thereof active ingredient (b), in an amount amount equivalent to from 0.125 mg to 22.5 mg, normally from 7.5 mg to 12.5 mg or more than 10 mg to 12.5 mg of pramipexole dihydrochloride monohydrate; and a statin active ingredient (c) selected from the group consisting of fluvastatin in an amount of from 20 mg to 40 mg; and lovastatin, in an amount per unit form of from 2.5 mg to 40 mg or from 20 mg to 40 mg, in admixture with a pharmaceutical carrier or vehicle in IR-formulation;
a domperidone Component (a), in an Unit Form which comprises a pharmaceutical composition comprising an active ingredient (a) selected from the group consisting of domperidone and pharmaceutically acceptable salts and solvates thereof, in an amount equivalent to from 2 mg to 120 mg of domperidone base, in admixture with a pharmaceutical carrier or vehicle in IR or ER formulation,
   for the treatment of a PMND in a patient in combination with Component (bc) in an Unit Form which comprises a pharmaceutical composition in dosage unit form comprising pramipexole or pharmaceutically acceptable salt or solvate thereof active ingredient (b), in an amount equivalent to from 0.375 mg to 45 mg, normally from 15 mg to 25 mg or from more than 20 mg to 25 mg of pramipexole dihydrochloride monohydrate; and a statin active ingredient (c) selected from the group consisting of fluvastatin in an amount of from 20 mg to 80 mg; and lovastatin, in an amount of from 2.5 mg to 80 mg or from 20 mg to 80 mg, normally from 20 mg to 60 mg, in admixture with a pharmaceutical carrier or vehicle in ER-formulation, to be administered once a day;
a Component (ab), as a Unit Form comprising a domperidone active ingredient (a) selected from the group consisting of domperidone and pharmaceutically acceptable salts and solvates thereof, in an amount equivalent to from 2 mg to 120 mg of domperidone base, and pramipexole or a pharmaceutically acceptable salt or solvate thereof active ingredient (b), in an amount equivalent to from 0.125 mg to 45 mg of pramipexole dihydrochloride monohydrate,
   for the treatment of a PMND in combination with Component (c), as a pharmaceutical composition comprising a statin, in an amount per unit form of from 0.5 mg to 80 mg, in admixture with a pharmaceutical carrier or vehicle;
a Component (ab), in an Unit Form which comprises a pharmaceutical composition comprising a domperidone active ingredient (a) selected from the group consisting of domperidone and pharmaceutically acceptable salts and solvates thereof, in an amount equivalent to from 2 mg to 60 mg of domperidone base, and pramipexole or a pharmaceutically acceptable salt or solvate thereof active ingredient (b), in an amount equivalent to from 0.125 mg to 22.5 mg, normally from 7.5 mg to 12.5 mg (or from more than 7.5 mg) or from more than 10 mg to 12.5 mg or from 10.125 mg to 12.5 mg of pramipexole dihydrochloride monohydrate, in IR-formulation,
   for the treatment of a PMND in combination with Component (c), in an Unit Form which comprises a pharmaceutical composition comprising a statin, in an amount of from 0.5 mg to 80 mg, in IR-formulation or ER-formulation;
a Component (ab), in an Unit Form which comprises a pharmaceutical composition comprising a domperidone active ingredient (a) selected from the group consisting of domperidone and pharmaceutically acceptable salts and solvates thereof, in an amount equivalent to from 2 mg to 120 mg of domperidone base, and pramipexole or a pharmaceutically acceptable salt or solvate thereof active ingredient (b), in an amount equivalent to from 0.125 mg to 45 mg, normally from 15 mg to 25 mg, from more than 20 mg to 25 mg or from 20.25 mg to 25 mg of pramipexole dihydrochloride monohydrate,
   for the treatment of a PMND in combination with Component (c), in an Unit Form which comprises a pharmaceutical composition comprising a statin, in an amount of from 0.5 mg to 80 mg, in admixture with a pharmaceutical carrier or vehicle;
a Component (ab), in an Unit Form which comprises a pharmaceutical composition comprising a domperidone active ingredient (a) selected from the group consisting of domperidone and pharmaceutically acceptable salts and solvates thereof, in an amount equivalent to from 4 mg to 120 mg of domperidone base, and pramipexole or a pharmaceutically acceptable salt or solvate thereof active ingredient (b), in an amount equivalent to from 0.375 mg to 45 mg, normally from 15 mg to 25 mg, from more than 20 mg to 25 mg or from 20.25 mg to 25 mg of pramipexole dihydrochloride monohydrate, in an ER-formulation to be administered once a day,
   for the treatment of a PMND in combination with Component (c), in an Unit Form which comprises a pharmaceutical composition comprising rosuvastatin calcium, in an amount of from 2.5 mg to 40 mg, in an IR-formulation to be administered once a day;
a Component (ac), in an Unit Form which comprises a pharmaceutical composition comprising a domperidone active ingredient (a) selected from the group consisting of domperidone and pharmaceutically acceptable salts and solvates thereof, in an amount equivalent to from 2 mg to 120 mg of domperidone base; and a statin active ingredient (c), in an amount per unit form of from 0.5 mg to 80 mg,
   for the treatment of a PMND in combination with Component (b), in an Unit Form which comprises a pharmaceutical composition comprising 6-propylamino-4,5,6,7-tetrahydro-1,3-benzothiazole-2-amine active ingredient (b), in an amount equivalent to from 0.125 mg to 3000 mg of pramipexole dihydrochloride monohydrate, including a (S)-enantiomer amount equivalent to from 0.125 mg to 45 mg, normally from 7.5 mg (or more than 7.5 mg) to 25 mg, from 15 mg to 25 mg, from more 20 mg to 25 mg or from 20.25 mg to 25 mg of pramipexole dihydrochloride monohydrate, in admixture with a pharmaceutical carrier or vehicle; and
a Component (ac), as an Unit Form comprising a domperidone active ingredient (a) selected from the group consisting of domperidone and pharmaceutically acceptable salts and solvates thereof, in an amount equivalent to from 2 mg to 120 mg of domperidone base, in an IR- or ER formulation; and a statin active ingredient (c) selected from the group consisting of atorvastatin, in an amount of from 10 mg to 80 mg, lovastatin, in an amount of from 20 mg to 60 mg; pravastatin, in an amount per unit form of from 10 mg to 80 mg; rosuvastatin calciun, in an amount of from 5 mg to 40 mg; and simvastatin, in an amount of from 5 mg to 80 mg, in an IR- or ER formulation,
   for the treatment of a PMND in combination with Component (b), as a pharmaceutical composition in dosage unit form comprising 6-propylamino-4,5,6,7-tetrahydro-1,3-benzothiazole-2-amine active ingredient, in an amount per unit form equivalent to from 0.125 mg to 3000 mg of pramipexole dihydrochloride monohydrate, including a (S)-enantiomer amount per unit form equivalent to from 0.125 mg to 45 mg normally from 7.5 mg (or more than 7.5 mg) to 25 mg, from 15 mg to 25 mg, from more than 20 mg to 25 mg or from 20.25 mg to 25 mg of pramipexole dihydrochloride monohydrate, in admixture with a pharmaceutical carrier or vehicle.

Preferably, said 6-propylamino-4,5,6,7-tetrahydro-1,3-benzothiazole-2-amine Component (b) comprises pramipexole or a pharmaceutically acceptable salt thereof, in an amount per unit form as illustrated in the “The 6-propylamino-4,5,6,7-tetrahydro-1,3-benzothiazole-2-amine Component (b)” section in admixture with a pharmaceutical carrier or vehicle, in an IR or ER formulation.

Preferably, according to this specific embodiment of the three aspects of the invention, the domperidone/6-propylamino-4,5,6,7-tetrahydro-1,3-benzothiazole-2-amine/statin combination comprises
a Component (ac), in a pharmaceutical composition comprising a domperidone active ingredient (a) selected from the group consisting of domperidone and pharmaceutically acceptable salts and solvates thereof, in an amount equivalent to from 2 mg to 60 mg of domperidone base; and lovastatin active ingredient (c), in an amount per unit form of from 2.5 mg to 40 mg, normally from 2.5 mg to 30 mg in IR-formulation, to be administered twice a day,
   for the treatment of a PMND in combination with Component (b), in a pharmaceutical composition comprising pramipexole active ingredient (b), in an amount equivalent to from 0.125 mg to 45 mg, normally from 7.5 mg (or more than 7.5 mg) to 25 mg, from 15 mg to 25 mg, from more than 20 mg to 25 mg or from 20.25 mg to 25 mg of pramipexole dihydrochloride monohydrate, in admixture with a pharmaceutical carrier or vehicle in an IR-formulation or ER-formulation, to be administered twice or, respectively, once a day.

According to a further specific embodiment, Component (ab), Component (ac), Component (bc) and the fixed-dose combination (abc) may be in a Unit Form wherein the domperidone, the 6-propylamino-4,5,6,7-tetrahydro-1,3-benzothiazole-2-amine (preferably pramipexole) and the statin are each in admixture with a pharmaceutical carrier or vehicle, in different formulations.

A fixed-dose combination (abc) may be in an Unit Form comprising a Component (a) selected from the group consisting of domperidone or a pharmaceutically acceptable salt thereof, in an amount equivalent to from 2 mg to 120 mg of domperidone base; a Component (b) selected from the group consisting of the racemate or a pharmaceutically acceptable salt thereof, in an amount per unit form equivalent to from 0.25 mg to 90 mg, normally from 30 mg to 50 mg or from more than 40 mg to 50 mg of pramipexole dihydrochloride monohydrate, pramipexole or a pharmaceutically acceptable salt thereof, in an amount equivalent to from 0.125 mg to 45 mg, normally from 15 mg to 25 mg or from more than 20 mg to 25 mg of pramipexole dihydrochloride monohydrate, and a (R)/(S)-mixture, in an amount per unit form of from 50 mg to 3000 mg, inclusive of a (S)-enantiomer amount per unit form equivalent to from 0.125 mg to 45 mg, normally from 7.5 mg (or from more than 7.5 mg) to 25 mg, from 15 mg to 25 mg, from more than 20 mg to 25 mg or from 20.25 mg to 25 mg of pramipexole dihydrochloride monohydrate; and at least one synergistic agent Component (c) selected from the group consisting of fluoxetine, in an amount (in fluoxetine base) of from 2 mg to 90 mg; zonisamide, in an amount of from 25 mg to 600 mg; and a statin, in an amount of from 0.5 mg to 80 mg.

A specific fixed-dose combination (abc) is in an Unit Form comprising
(a) a domperidone selected from the group consisting of domperidone base, domperidone maleate and domperidone succinate (1:1), in an amount equivalent to from 2 mg to 120 mg of domperidone base;
(b) pramipexole dihydrochloride monohydrate, in an amount of from 0.125 mg to 45 mg, normally from 7.5 mg (or from more than 7.5 mg) to 25 mg, from 15 mg to 25 mg, from more than 20 mg to 25 mg or from 20.25 mg to 25 mg;
(c) fluoxetine hydrochloride, in an amount per unit form equivalent to from 2 mg to 90 mg of fluoxetine base,
   and a pharmaceutical carrier or vehicle.

A specific fixed-dose combination (abc) is in an Unit Form comprising
(a) a domperidone selected from the group consisting of domperidone base, domperidone maleate and domperidone succinate (1:1), in an amount per unit form equivalent to from 2 mg to 120 mg of domperidone base;
(b) pramipexole dihydrochloride monohydrate, in an amount of from 0.125 mg to 45 mg, normally from 7.5 mg (or from more than 7.5 mg) to 25 mg, from 15 mg to 25 mg, from more than 20 mg to 25 mg or from 20.25 mg to 25 mg; and
(c) zonisamide free acid, in an amount of from 25 mg to 600 mg,
   in admixture with a pharmaceutical carrier or vehicle.

An advantageous Unit Form (abc) comprises or consists of a pharmaceutical composition comprising a domperidone active ingredient Component (a), in an amount equivalent to from 2 mg to 120 mg of domperidone base; a 6-propylamino-4,5,6,7-tetrahydro-1,3-benzothiazole-2-amine active ingredient (b), in an amount equivalent to from 0.125 mg to 3000 mg of pramipexole dihydrochloride monohydrate, including a (S)-enantiomer amount equivalent to from 0.125 mg to 45 mg, normally from 7.5 mg (or from more than 7.5 mg) to 25 mg, from 15 mg to 25 mg, from more than 20 mg to 25 mg or from 20.25 mg to 25 mg of pramipexole dihydrochloride monohydrate; and a statin active ingredient Component (c), in an amount of from 0.5 mg to 80 mg, in admixture with a pharmaceutical carrier or vehicle.

A typical composition (abc) comprises or consists of a Unit Form comprising
(a) domperidone in an amount of from 2 mg to 120 mg;
(b) 6-propylamino-4,5,6,7-tetrahydro-1,3-benzothiazole-2-amine selected from the group consisting of the racemate or a pharmaceutically acceptable salt thereof, in an amount equivalent to from 0.25 mg to 90 mg, normally from 15 mg (or from more than 15 mg) to 50 mg, from 30 mg to 50 mg, from more than 40 mg to 50 mg or from 40.5 mg to 50 mg of pramipexole dihydrochloride monohydrate; pramipexole or a pharmaceutically acceptable salt thereof, in an amount equivalent to from 0.125 mg to 45 mg, normally from 7.5 mg (or from more than 7.5 mg) to 25 mg, from 15 mg to 25 mg, from more than 20 mg to 25 mg or from 20.25 mg to 25 mg of pramipexole dihydrochloride monohydrate, and a (R)/(S)-mixture, in an amount equivalent to from 50 mg to 3000 mg, inclusive of a (S)-enantiomer amount equivalent to from 0.125 mg to 45 mg, normally from 7.5 mg (or from more than 7.5 mg) to 25 mg, from 15 mg to 25 mg, from more than 20 mg to 25 mg or from 20.25 mg to 25 mg of pramipexole dihydrochloride monohydrate;
(c) a statin, in an amount of from 0.5 mg to 80 mg; and
   a pharmaceutical carrier or vehicle.

In an Unit Form Component (a), Component (ab), Component (ac), Component (bc) or Component (abc), said domperidone, when present in the above, pharmaceutical compositions, is in an amount equivalent to a range selected from the group consisting of from 2 mg to 120 mg, from 2 mg to 100 mg, from 2 mg to 80 mg, from 2 mg to 60 mg, from 2 mg to 40 mg, from 2 mg to 30 mg or from 2 mg to 20 mg, normally from 10 mg to 120 mg, from 10 mg to 100 mg, from 10 mg to 80 mg, from 10 mg to 60 mg, from 10 mg to 40 mg, from 10 mg to 30 mg, or from 10 mg to 20 mg of domperidone base.

In an Unit Form, Component (a), Component (ab), Component (ac), Component (bc) or Component (abc), said 6-propylamino-4,5,6,7-tetrahydro-1,3-benzothiazole-2-amine, preferably pramipexole, Component (b) when present in the above pharmaceutical compositions, is in an amount equivalent to a range selected from the group consisting of from 0.125 mg to 3000 mg of pramipexole dihydrochloride monohydrate, including a (S) enantiomer amount equivalent to from 0.125 mg to 45 mg of pramipexole dihydrochloride monohydrate, said. 6-propylamino-4,5,6,7-tetrahydro-1,3-benzothiazole-2-amine preferably being pramipexole, in an amount equivalent to a range selected from the group consisting of from 0.125 mg to 45 mg, from 1.5 mg to 45 mg, from 1.625 mg to 45 mg, from 3 mg to 45 mg, from more than 4.5 mg to 45 mg, from 4.8 mg to 45 mg, from more than 6 mg to 45 mg, from 7,5 mg to 45 mg, from more than 10 mg to 45 mg, from 14.5 mg to 45 mg, from 15 mg to 45 mg, from 15 mg to 40 mg, from 15 mg to 35 mg, from 15 mg to 30 mg, from 15 mg to 25 mg and from 15 mg to 20 mg of pramipexole dihydrochloride monohydrate. According to an advantageous embodiment, preamipexole is present in an Unit Form Component (a), Component (ab), Component (ac), Component (bc) or Component (abc) in an an amount (in pramipexole dihydrochloride monohydrate) of from more than 20 mg to 25 mg, normally from 20.25 mg to 25 mg.

In an Unit Form Component (a), Component (ab), Component (ac), Component (bc) or Component (abc), said statin Component (c), when present in the above pharmaceutical compositions, is in an amount of from 0.5 mg to 80 mg and is preferably selected from the group consisting of atorvastatin and pharmaceutically acceptable salts and solvates thereof, in an amount equivalent to from 5 mg to 80 mg, of atorvastatin free acid; fluvastatin and pharmaceutically acceptable salts and solvates thereof, in an amount equivalent to from to 10 mg to 40 mg of fluvastatin free acid; lovastatin, in an amount of from 2.5 mg to 80 mg; pitavastatin and pharmaceutically acceptable salts and solvates thereof, in an amount equivalent to from 0.5 mg to 4 mg of pitavastatin free acid; pravastatin and pharmaceutically acceptable salts and solvates thereof, in an amount of from 5 mg to 80 mg of pravastatin sodium; rosuvastatin and pharmaceutically acceptable salts and solvates thereof, in an amount equivalent to from 2.5 mg to 40 mg, normally from 2.5 mg to 30 mg, of rosuvastatin calcium; and simvastatin, in an amount of from 2.5 mg to 80 mg

Alternatively, in the Unit Form, said domperidone is selected from the group consisting of domperidone base, domperidone maleate and domperidone succinate (1:1) in an amount equivalent to a range selected from the group consisting of from 4 mg to 120 mg, from 4 mg to 100 mg, from 4 mg to 80 mg, from 4 mg to 60 mg, from 4 mg to 40 mg, from 4 mg to 30 mg or from 4 mg to 20 mg, normally from 40 mg to 120 mg, from 40 mg to 100 mg, from 40 mg to 80 mg, from 40 mg to 60 mg, and from 40 mg to 50 mg of domperidone base in an ER-formulation.

In the Unit Form, said 6-propylamino-4,5,6,7-tetrahydro-1,3-benzothiazole-2-amine Component (b) is preferably selected from the group consisting of pramipexole and pharmaceutically acceptable salts and solvates thereof, in an amount per IR-unit form equivalent to a range selected from the group consisting of from 0.125 mg to 30 mg, from 0.125 mg to 22.5 mg, from 0.125 mg to 11.25 mg, from 0.125 mg to 15 mg, from 0.125 mg to 20 mg, and from 0.125 mg to 10 mg of pramipexole dihydrochloride monohydrate, depending on safety and tolerability, in combination with the domperidone Component (a) and with the statin Component (c). This Unit Form includes low doses to be used in pediatric patients or during the titration period.

Advantageously, said pramipexole or a pharmaceutically acceptable salt or solvate thereof, is present in an amount per IR unit form equivalent to a range selected from the group consisting of from 3 mg to 22.5 mg, from more than 4.5 mg to 22.5 mg, from 4.8 mg to 22.5 mg, from more than 6 mg to 22.5 mg, from 7.5 mg to 22.5 mg, and from more than 10 mg to 22.5 mg of pramipexole dihydrochloride monohydrate.

Preferably, said pramipexole or pharmaceutically acceptable salt or solvate thereof active ingredient, is in an amount per IR unit form equivalent to a range selected from the group consisting of from 7.5 mg to 22.5 mg, from more than 10 mg to 22.5 mg, from 14.5 mg to 22.5 mg, from 15 mg to 22.5 mg, from 17.5 mg to 22.5 mg and from 20 mg to 22.5 mg of pramipexole dihydrochloride monohydrate.

Normally, in an Unit Form in IR-formulation, said pramipexole or pharmaceutically acceptable salt or solvate thereof active ingredient, is present in an amount equivalent to from 7.5 mg to 12.5 mg or from more than 10 mg to 12.5 mg. In particular, in said IR-formulation pramipexole is present in an amount equivalent to from 10.125 mg to 12.5 mg of pramipexole dihydrochloride monohydrate.

According to a particular embodiment, as also described in “The formulations” section below, said 6-propylamino-4,5,6,7-tetrahydro-1,3-benzothiazole-2-amine is pramipexole or a pharmaceutically acceptable salt or solvate thereof, in an amount per ER unit form equivalent to a range selected from the group consisting of from more than 4.5 mg to 45 mg, from 4.8 mg to 45 mg, from more than 6 mg to 45 mg, from more than 6.5 mg to 45 mg, and from more than 10 mg to 45 mg of pramipexole dihydrochloride monohydrate.

Said pramipexole or pharmaceutically acceptable salt or solvate thereof active ingredient may also be present in an amount per ER unit form equivalent to a range selected from the group consisting of from more than 10 mg to 45 mg, from 14.5 mg to 45 mg, from 15 mg to 45 mg, from 15 mg to 40 mg, from 15 mg to 35 mg, from 15 mg to 30 mg, from 15 mg to 25 mg, from more than 20 mg to 25 mg and from 20.25 mg to 25 mg of pramipexole dihydrochloride monohydrate. A particularly advantageous Unit Form in ER-formulation comprises pramipexole in an amount (in pramipexole dihydrochloride monohydrate) of from more than 20 mg to 25 mg, normally from 20.25 mg to 25 mg.

In the combination with domperidone and with 6-propylamino-4,5,6,7-tetrahydro-1,3-benzothiazole-2-amine, the statin Component (c) is present in the Unit Form in an amount of from 0.5 mg to 80 mg. Preferably, in Component (c), Component (ac), Component (bc) or Component (abc), said statin active ingredient is selected from the group consisting of atorvastatin, in an amount of from 10 mg to 80 mg, lovastatin, in an amount of from 2.5 mg to 80 mg or from 20 mg to 60 mg; pravastatin, in an amount per unit form of from 10 mg to 80 mg; rosuvastatin calciun, in an amount of from 5 mg to 40 mg; and simvastatin, in an amount of from 5 mg to 80 mg.

According to a further specific embodiment, Component (ab), Component (ac), Component (bc) and the fixed-dose combination (abc) may be in a Unit Form wherein the domperidone, the 6-propylamino-4,5,6,7-tetrahydro-1,3-benzothiazole-2-amine (preferably pramipexole) and the statin are each in admixture with a pharmaceutical carrier or vehicle, in different formulations.

In the case of separate (concurrent or sequential) administration of a domperidone/pramipexole/fluoxetine, zonisamide or statin combination, said domperidone Component (a), in an effective amount per unit form, said pramipexole Component (b), in an effective amount per unit form, and said fluoxetine, zonisamide, or statin Component (c), in an effective amount per unit form, can each be packaged in a kit comprising said domperidone, in admixture with a pharmaceutical carrier or vehicle, in a container; said pramipexole, in admixture with a pharmaceutical carrier or vehicle, in another, separate container, and said fluoxetine, zonisamide, or statin, in admixture with a pharmaceutical carrier or vehicle, in a third, separate container.

Similarly, for example, a domperidone/pramipexole/statin combination may be packaged in kit wherein a pharmaceutical composition comprising domperidone Component (a), in an amount per unit form of from 2 mg to 60 mg or from 2 mg to 20 mg in an IR oral formulation is in a container, a pharmaceutical composition comprising pramipexole dihydrochloride monohydrate Component (b), in an amount per unit form of from 0.375 mg to 45 mg, normally from 15 mg to 25 mg, from more than 20 mg to 25 mg or from 20.25 mg to 25 mg, in an ER oral formulation is in another container; and a pharmaceutical composition comprising rosuvastatin calcium Component (c), in an amount per unit form of from 2.5 mg to 40 mg in an IR oral formulation is in third, separate container.

A domperidone/pramipexole/zonisamide combination may be packaged for example in kit wherein a pharmaceutical composition comprising a fixed-dose combination Component (ac) comprising domperidone, in an amount (in domperidone base) of from 2 mg to 60 mg, and a zonisamide amount (in zonisamide free acid) of from 25 mg to 200 mg in an IR oral formulation, is in a container; and a pharmaceutical composition comprising pramipexole dihydrochloride monohydrate Component (b), in an amount per unit form of from 0.375 mg to 45 mg, normally from 15 mg to 25 mg, from more than 20 mg to 25 mg or from 20.25 mg to 25 mg, in an ER oral formulation, is in another container.

### The Formulations

For the intended use in the treatment of a PMND in combination with 6-propylamino-4,5,6,7-tetrahydro-1,3-benzothiazole-2-amine, preferably pramipexole, and a statin, the domperidone Component (a) is formulated in a pharmaceutical composition, wherein said domperidone is in admixture with a pharmaceutical carrier or vehicle. For said treatment, also the 6-propylamino-4,5,6,7-tetrahydro-1,3-benzothiazole-2-amine, preferably pramipexole, Component (b) is formulated in a pharmaceutical composition, wherein said 6-propylamino-4,5,6,7-tetrahydro-1,3-benzothiazole-2-amine, preferably pramipexole, is in admixture with a pharmaceutical carrier or vehicle. Similarly, for said treatment, the statin Component (c) is formulated in a pharmaceutical composition in dosage unit form in admixture with a pharmaceutical carrier or vehicle.

The dosage, i.e. the amount of active ingredient in a single dose (amount per unit form) to be administered to the patient, can vary widely depending on the age, weight, and the health condition of the patient. This dosage includes the administration of a domperidone amount from 2 mg to 120 mg, according to the potency of domperidone and the age of the patient, an effective amount of 6-propylamino-4,5,6,7-tetrahydro-1,3-benzothiazole-2-amine, preferably a pramipexole amount equivalent to from 0.125 mg to 45 mg of pramipexole dihydrochloride monohydrate, according to the age of the patient, and an amount of at least one component (c) selected from the group consisting of a fluoxetine amount equivalent to from 2 mg to 80 mg of fluoxetine base, a zonisamide amount of from 25 mg to 600 mg, or a statin amount of from 0.5 mg to 80 mg, according to the potency of the statin and the age of the patient, from one to three times a day by intravenous, subcutaneous, oral, or transcutaneous administration, according to the strength of the doses of the each of the active ingredients.

The above pharmaceutical compositions are formulated in admixture with a pharmaceutical carrier or vehicle for any administration route. For example, said pharmaceutical compositions are in a pharmaceutical dosage unit form for oral, intravenous (including infusion), intramuscular, intranasal, intraperitoneal, subcutaneous, transdermal, or rectal administration.

In the pharmaceutical compositions of the present invention for oral, subcutaneous, intravenous, transdermal or topical administration, the active ingredients are preferably administered in the form of dosage units, comprising a predetermined amount of active ingredient per unit form, in admixture with the classic excipients suitable for different ways of administration, as described above.

These unit forms are manufactured according to conventional technologies. Particularly advantageous are the formulations in the form of tablets, multi-score tablets, multi-layer tablets, coated tables, orally disintegrating tablets, extended release tablets, hard or soft capsules, multi-compartment capsules, extended-release capsules, suppositories for rectal administration, patches for transdermal administration, liquid oral solutions, syrups or suspensions in a predetermined unit form, apparatus for intravenous infusion, and vials for the intravenous or subcutaneous administration.

The pharmaceutical compositions may be formulated in oral unit forms such as tablets or gelatin capsules wherein the domperidone Component (a), the 6-propylamino-4,5,6,7-tetrahydro-1,3-benzothiazole-2-amine Component (b), the fluoxetine, zonisamide, or statin Component (c), the Component (ab), the Component (ac), the Component (bc) and, respectively, the Component (abc) active ingredients are in admixture with a carrier or vehicle that may include a diluent, such as cellulose, microcrystalline cellulose, a starch such as maize or corn starch, dextrose, lactose, mannitol, sorbitol or sucrose; a lubricant, such as, acid, calcium stearate, magnesium stearate, polyethylene glycol, silica, colloidal silicon dioxide or talc; an emulsifying agent such as silicone, sorbitan monooleate, glyceryl monostearate or sodium lauryl sulfate; and if needed, a binder such as magnesium aluminum silicate, gelatin, methylcellulose, sodium carboxymethylcellulose, a pregelatinized starch such as pregelatinized potato starch, or polyvinylpyrrolidone.

Said oral unit forms may be tablets coated with sucrose, iron oxide, titanium dioxide or with various polymers for an immediate release.

Alternatively, the tablets can be manufactured by using carriers such as acrylic and methacrylic acid polymers and copolymers; cellulose derivatives such as hydroxypropylethylcellulose; or other appropriate materials having a prolonged or delayed activity by progressively releasing a predetermined quantity of active ingredient.

For example, the unit forms may be formulated in tablets in which each of the Components (a), (b), (c), (ab), (ac), (bc) and, respectively, the fixed-dose combination (abc) is in ER-formulation, for example in admixture with hydroxypropyl methyl cellulose or in a film-coated microgranule. Carriers and vehicles for ER-tablets include retardant materials such as acrylic and methacrylic acid polymers and copolymers; the aforementioned cellulose derivatives such as hydroxypropylmethylcellulose (hypromellose), hydroxyethylcellulose, hydroxypropylethylcellulose, hydroxypropylcellulose, methylcellulose, ethylcellulose, or sodium carboxymethylcellulose; gums; waxes; glycerides or aliphatic alcohols or a mixture thereof.

These unit forms may also be manufactured according to conventional technologies allowing, for example, an ER-formulation of the domperidone Component (a) and an IR-formulation of rosuvastatin Component (c) in the same unit-form Component (ac).

Syrups and orally dispersible tablets may also comprise sweeteners, lubricants, taste-masking agents, binders, and coloring agents.

Suppositories are manufactured by using a suppository base such as cocoa butter, poloxamers combined with solvents such as. polyethylene glycols (for example PEG 3350), propylene glycol, or triglycerides according to conventional technologies.

A Transdermal drug delivery system (TDDS) provides transdermal delivery using transdermal drug formulations and transdermal patches incorporating such transdermal drug formulations. A transdermal drug delivery system may include a composition in form of a patch, a cream, a gel, a lotion or a paste comprising for example a domperidone, pramipexole or both the active ingredients; a domperidone, a statin or both the active ingredients, or a domperidone, pramipexole and statin altogether.

A typical TDDS is a patch formulation wherein the active ingredient or the mixture of the active ingredients may comprise adjuvants such as D-sorbitol, gelatin, kaolin, methyl paraben, polysorbate 80, propylene glycol, propyl paraben, povidone, polyvinylalcohol, polyethyleneglycol, sodium carboxymethylcellulose, hydroxypropylmethylcellulose, sodium polyacrylate, polymethacrylate, tartaric acid, titanium dioxide, and purified water. A patch formulation may also contain skin permeability enhancer such as lactate esters (e.g., lauryl lactate), triacetin or diethylene glycol monoethyl ether.

Patch formulations comprising domperidone hydrochloride or domperidone maleate are described in the aforementioned Latha et al. 2012 and, respectively, Shirisha et al. 2017 references. Patch formulations comprising domperidone base are described by Prabhu et al 2011 and, as a bilayered matrix-type patch, by Madishetti et al 2010.

A patch formulation comprising pramipexole hydrochloride in a hydrophilic substrate such as polyvinyl alcohol, polyvinyl pyrrolidone, polyacrylate, polyacrylamide or mixtures thereof is described in CN 103610666.

Unit forms may be formulated in tablets in which Component (b) and Component (c) are each in ER-formulation, for example each in admixture with hydroxypropyl methyl cellulose or in a film-coated microgranule. These unit forms (b) and (c) are concurrently or sequentially administered to a patient suffering from a PMND in combination with an oral unit form such as a tablet or gelatin capsule wherein domperidone Component (a) is formulated with a diluent and a lubricant in an IR-formulation.

Unit forms may be formulated in tablets in which Component (b) and Component (c) each in ER-formulation, for example pramipexole and lovastatin, each in admixture with hydroxypropyl methyl cellulose or in a film-coated microgranule. These unit forms (b) and (c) are concurrently or sequentially administered to a patient suffering from a PMND in combination with an oral unit form such as a tablet or gelatin capsule wherein Component (a) is formulated with a diluent and a lubricant in an IR-formulation, or in a tablet or capsule for extended release.

Similarly, Unit Forms may be formulated in tablets in which Component (a) and Component (c), for example domperidone and lovastatin, are each in ER-formulation, for example each in admixture with hydroxypropyl methyl cellulose or in a film-coated microgranule to be administered once a day, or each in an IR-formulation for example each in admixture with a diluent and a lubricant to be administered twice a day. These unit forms (a) and (c) are concurrently or sequentially administered to a patient suffering from a PMND in combination with an oral unit form such as a tablet or gelatin capsule wherein pramipexole Component (b) is formulated with a diluent and a lubricant in an IR-formulation or with a retardant material in ER-formulation.

As set forth above, said oral unit forms may also be tablets or capsules comprising Component (ab), Component (ac) or Component (bc), wherein one of the active ingredient is in an IR-formulation and another one is in an ER-formulation.

For example a Component (ac) is an unit form comprising domperidone base in an ER-formulation and rosuvastatine calcium in IR-formulation, each in the respective amount per unit form as described above, to be administered once a day in combination with an unit form comprising pramipexole, in an IR-formulation to be administered once a day or in an ER-formulation to be administered once a day.

As another example, a Component (a) is an unit form comprising domperidone base or domperidone hydrochloride, in an amount per unit form as described above, in IR-formulation to be administered twice a day, in combination, including fixed-dose combinations (abc), with an unit form Component (bc) to be administered once a day, comprising a fixed-dose combination of pramipexole dihydrochloride monohydrate in ER-formulation and rosuvastatin calcium in IR-formulation, each in an amount per unit form as described above.

Preferably, in Component (ab), domperidone and pramipexole, each in an amount per unit form as described above, are both in an IR-formulation.

In particular, in said Component (ac) (ab) said domperidone is in an amount (in domperidone base) of from 2 mg to 60 mg, and said pramipexole pramipexole dihydrochloride monohydrate is in IR-formulation comprising said pramipexole dihydrochloride monohydrate in an amount of from 0.125 mg to 22.5 mg, normally from 7.5 mg to 12.5 mg, from more than 10 mg to 12.5 mg or from 10.125 mg to 12.5 mg. Said Component (ab) is administered twice a day to a patient suffering from a PMND in combination with at leaste one of fluoxetine, zonisamide or a statin Component (c), in an IR-formulation or ER-formulation, administered once or twice a day. Said Component (c) IR-formulation may comprise fluoxetine, in an amount equivalent to from 2 mg to 40 mg or from 2 mg to 45 mg, to be administered once or twice a day. Said Component (c) ER-formulation may comprise fluoxetine, in an amount equivalent to from 20 mg to 90 mg of fluoxetine base, to be administered once a day. Said Component (c) may be in the specific fluoxetine 90 mg ER-weekly preparation to be administered once a week. Alternatively. said Component (c) may be an IR-formulation comprising zonisamide, in an amount of from 25 mg to 200 mg to be administered once to three times per day; or an ER-formulation comprising zonisamide, in an amount of from 25 mg to 600 mg, normally from 200 mg to 600 mg, to be administered once a day.

Similarly, said unit form may comprise domperidone in an IR-formulation or ER-formulation and fluoxetine hydrochloride also in an IR-formulation or ER-formulation, each in the amount per unit form as described above, in a Component (ac) fixed dose combination, to be administered in combination with pramipexole Component (b), in IR-formulation or ER-formulation. Said unit form may comprise domperidone in an ER-formulation and fluoxetine hydrochloride in an ER-formulation, each in the amount per unit form as described above, in a Component (ac) fixed dose combination, to be administered in combination with pramipexole Component (b), in IR-formulation or ER-formulation.

Similarly, an oral unit form may comprise domperidone, in an amount per unit form (in domperidone base) of form of from 2 mg to 60 mg an IR-formulation and zonisamide, in an amount per unit form (in zonisamide free acid) of from 25 mg to 200 mg, in an IR-formulation, in a Component (ac) fixed dose combination, to be administered in combination with pramipexole, in IR-formulation of ER-formulation.

Similarly, an oral unit form may comprise domperidone, in an amount per unit form (in domperidone base) of form of from 2 mg to 60 mg an IR-formulation and lovastatin, in an amount per unit form of from 10 mg to 40 mg, in an IR-formulation, in a Component (ac) fixed dose combination, to be administered in combination with pramipexole, in IR-formulation of ER-formulation.

In a domperidone/pramipexole/fluoxetine combination, an Unit Form Component (ac) may be manufactured by using a two-layer tablet or two-compartment capsule wherein domperidone Component (a), in an amount (in domperidone base) of from 2 mg to 60 mg, in admixture with a pharmaceutical carrier for immediate release, is in the first layer or compartment; and fluoxetine Component (c), in an amount of from 2 mg to 45 mg, normally from 2 mg to 40 mg, in admixture with a pharmaceutical carrier for immediate release is in the second layer or compartment. This Unit Form may be administered to a patient suffering from a PMND twice a day in combination with a Unit Form Component (c) manufactured
either in a tablet or capsule comprising a pharmaceutical carrier or vehicle for immediate release and pramipexole, in an amount (as pramipexole dihydrochloride monohydrate) of from 0.125 mg to 22.5 mg, to be administered to said patient twice a day;
or in a tablet or capsule or TDDS, for extended release, comprising a pharmaceutical carrier or vehicle and pramipexole, in an amount (as pramipexole dihydrochloride monohydrate) delivering from 0.325 mg to 45 mg once a day (in the case of a TDDS, over 24 hours).

In a similar domperidone/pramipexole/fluoxetine combination, a Unit Form Component (ab) may be manufactured by using IR-formulated domperidone, in an amount (in domperidone base) of from 2 mg to 60 mg in a first layer or compartment and an IR-formulated pramipexole amount (in in pramipexole dihydrochloride monohydrate) of from 0.125 mg to 22.5 mg, preferably from 7.5 mg to 22.5 mg, from 7.5 to 12.5 mg or from 10.125 mg to 12.5 mg, in the second layer or compartment. This Unit Form may be administered to a patient suffering from a PMND twice a day in combination with a Unit Form Component (c), in an IR-formulation comprising fluoxetine an amount (in fluoxetine base) of from 2 mg to 45 mg, normally from 2 mg to 40 mg, to be administered twice a day, in ER-formulation comprising fluoxetine an amount (in fluoxetine base) of from 4 mg to 90 mg, normally from 4 mg to 80 mg, to be administered once a day, or with the specific 90 mg ER-weekly preparation.

In the domperidone/pramipexole/zonisamide combination, an Unit Form Component (ac) may be manufactured using a two-layer tablet or two-compartment capsule wherein domperidone Component (a), in an amount (in domperidone base) of from 10 mg to 60 mg, in admixture with a pharmaceutical carrier for immediate release, is in the first layer or compartment; and zonisamide Component (c), in an amount of from 25 mg to 200 mg, in admixture with a pharmaceutical carrier for immediate release is in the second layer or compartment. This Unit Form may be administered to a patient suffering from a PMND twice a day in combination with a Unit Form Component (b) manufactured
either in a tablet or capsule comprising a pharmaceutical carrier or vehicle for immediate release and pramipexole, in an amount (as pramipexole dihydrochloride monohydrate) of from 0.125 mg to 22.5 mg, to be administered to said patient twice a day;
or in a tablet or capsule or TDDS, for extended release, comprising a pharmaceutical carrier or vehicle and pramipexole, in an amount (as pramipexole dihydrochloride monohydrate) delivering from 0.325 mg to 45 mg once a day (in the case of a TDDS, over 24 hours).

In a similar domperidone/pramipexole/zonisamide combination, an Unit Form Component (ab) may be manufactured by using IR-formulated domperidone Component (a), in an amount (in domperidone base) of from 2 mg to 60 mg in a first layer or compartment and an IR-formulated pramipexole Component (b) amount (in in pramipexole dihydrochloride monohydrate) of from 0.125 mg to 22.5 mg, normally from 7.5 mg to 22.5 mg, from 7.5 to 12.5 mg or from 10.125 mg to 12.5 mg, in the second layer or compartment. This Unit Form may be administered to a patient suffering from a PMND twice a day in combination with a Unit Form Component (c), in an IR-formulation comprising zonisamide free acid an amount of from 25 mg to 300 mg, normally from 25 mg to 200 mg, to be administered twice a day, or in ER-formulation comprising zonisamide free acid in an amount of from 25 mg to 600 mg, normally from 100 mg to 400 mg, to be administered once a day.

In oral unit forms of a domperidone/pramipexole/statin combination, domperidone Component (a) may present in an amount per unit form equivalent to a range selected from the group consisting of from 2 mg to 60 mg, from 2 mg to 50 mg, from 2 mg to 40 mg, from 2 mg to 30 mg, from 2 mg to 20 mg, from 2 mg to 15 mg or from 2 mg to 10 mg, normally from 10 mg to 60 mg, from 10 mg to 50 mg, from 10 mg to 40 mg, from 10 mg to 30 mg, and from 10 mg to 20 mg, of domperidone base in an IR-formulation; or in an amount equivalent to a range selected from the group consisting of from 4 mg to 120 mg, from 4 mg to 100 mg, from 4 mg to 80 mg, from 4 mg to 60 mg, from 4 mg to 40 mg, from 4 mg to 30 mg or from 4 mg to 20 mg, normally from 40 mg to 120 mg, from 40 mg to 100 mg, from 40 mg to 80 mg, from 40 mg to 60 mg, and from 40 mg to 50 mg of domperidone base in an ER-formulation.

In said unit forms, pramipexole or a pharmaceutically acceptable salt or solvate thereof Component (b) is present in an amount per unit form equivalent to a range selected from the group consisting of from 0.125 mg to 30 mg, from 0.125 mg to 22.5 mg, from 0.125 mg to 20 mg, from 0.125 mg to 15 mg, from 0.125 mg to 11.25 mg, and from 0.125 mg to 10 mg, advantageously from 3 mg to 22.5 mg, from more than 4.5 mg to 22.5 mg, from 4.8 mg to 22.5 mg, from more than 6 mg to 22.5 mg, from 7.5 mg to 22.5 mg, from more than 10 mg to 22.5 mg, from 14.5 mg to 22.5 mg, from 15 mg to 22.5 mg, from 17.5 mg to 22.5 mg, from and from 20 mg to 22.5 mg of pramipexole dihydrochloride monohydrate, in an IR-formulation; or in an amount equivalent to a range selected from the group consisting of from 0.375 mg to 45 mg, from 0.375 mg to 40 mg, from 0.375 mg to 30 mg, from 0.375 mg to 25 mg, and from 0.375 mg to 20 mg, advantageously from more than 4.5 mg to 45 mg, from 4.8 mg to 45 mg, from more than 6 mg to 45 mg, from more than 10 mg to 45 mg, from 14.5 mg to 45 mg, from 15 mg to 45 mg, from more than 20 mg to 45 mg and from 20.25 mg to 45 mg, normally in an amount equivalent to a range selected from the group consisting of from 15 mg to 25 mg, from more than 20 mg to 25 mg or form 20.25 mg to 25 mg of pramipexole dihydrochloride monohydrate, in an ER-formulation.

In said unit forms, the statin Component (c) may be
atorvastatin calcium trihydrate, present in an amount equivalent to from 5 mg to 80 mg, preferably from 10 mg to 60 mg, of atorvastatin free acid; fluvastatin sodium, present in an amount equivalent to from 10 mg to 40 mg, preferably from 20 mg to 30 mg, of fluvastatin free acid; lovastatin, present in an amount of from 2.5 mg to 40 mg, preferably from 20 mg to 30 mg; pitavastatin calcium, present in an amount equivalent to from 0.5 mg to 4 mg, preferably from 1 mg to 3 mg, of pitavastatin free acid; pravastatin sodium, present in an amount of from 5 mg to 80 mg, preferably from 10 mg to 60 mg; rosuvastatin calcium, present in an amount of from 2.5 mg to 40 mg, preferably from 2.5 mg to 30 mg; simvastatin, present in an amount of from 5 mg to 80, preferably from 2.5 mg to 60 mg,

### in IR-formulation; or

fluvastatin sodium, present in an amount equivalent to from 30 mg to 80 mg, preferably from 60 mag to 80 mg of fluvastatin free acid; or lovastatin, present in an amount of from 20 mg to 80 mg or from 40 mg to 80 mg, preferably from 40 mg to 60 mg,

### in ER-formulation.

For example, a Component (ac) is a unit form comprising domperidone or a pharmaceutically acceptable salt or solvate thereof, in an amount per unit form of from 4 mg to 120 mg in an ER-formulation and rosuvastatin calcium, in an amount per unit form of from 0.5 mg to 40 mg, in IR-formulation.

Similarly, an unit form may comprise pramipexole dihydrochloride monohydrate Component (b), in an amount of from 0.375 mg to 45 mg in an ER-formulation, and rosuvastatin calcium, in an amount of from 0.5 mg to 40 mg in IR-formulation, in a Component (bc) fixed dose combination, to be administered in combination with domperidole or a pharmaceutically acceptable salt or solvate thereof, in IR-formulation or ER-formulation.

The above Component (ac) and Component (bc) may be formulated in a two-layer tablet or in a two-compartment capsule.

In a domperidone/pramipexole/fluoxetine combination, an Unit Form Component (ac) may be manufactured by using a two-layer tablet or two-compartment capsule wherein domperidone Component (a), in an amount (in domperidone base) of from 2 mg to 60 mg, in admixture with a pharmaceutical carrier for immediate release, is in the first layer or compartment; and fluoxetine Component (c), in an amount of from 2 mg to 45 mg, normally from 2 mg to 40 mg, in admixture with a pharmaceutical carrier for immediate release is in the second layer or compartment. This Unit Form may be administered to a patient suffering from a PMND twice a day in combination with a Unit Form Component (c) manufactured
either in a tablet or capsule comprising a pharmaceutical carrier or vehicle for immediate release and pramipexole, in an amount (as pramipexole dihydrochloride monohydrate) of from 0.125 mg to 22.5 mg, to be administered to said patient twice a day;
or in a tablet or capsule or TDDS, for extended release, comprising a pharmaceutical carrier or vehicle and pramipexole, in an amount (as pramipexole dihydrochloride monohydrate) delivering from 0.325 mg to 45 mg once a day (in the case of a TDDS, over 24 hours).

In a similar domperidone/pramipexole/fluoxetine combination, an Unit Form Component (ab) may be manufactured by using IR-formulated domperidone, in an amount (in domperidone base) of from 2 mg to 60 mg in a first layer or compartment and an IR-formulated pramipexole amount (in in pramipexole dihydrochloride monohydrate) of from 0.125 mg to 22.5 mg, preferably from 7.5 mg to 22.5 mg, from 7.5 to 12.5 mg or from 10.125 mg to 12.5 mg, in the second layer or compartment. This Unit Form may be administered to a patient suffering from a PMND twice a day in combination with a Unit Form Component (c), in an IR-formulation comprising fluoxetine an amount (in fluoxetine base) of from 2 mg to 45 mg, normally from 2 mg to 40 mg, to be administered twice a day, in ER-formulation comprising fluoxetine an amount (in fluoxetine base) of from 4 mg to 90 mg, normally from 4 mg to 80 mg, to be administered once a day, or with the specific 90 mg ER-weekly preparation.

In the domperidone/pramipexole/zonisamide combination, an Unit Form Component (ac) may be manufactured using a two-layer tablet or two-compartment capsule wherein domperidone Component (a), in an amount (in domperidone base) of from 10 mg to 60 mg, in admixture with a pharmaceutical carrier for immediate release, is in the first layer or compartment; and zonisamide Component (c), in an amount of from 25 mg to 200 mg, in admixture with a pharmaceutical carrier for immediate release is in the second layer or compartment. This Unit Form may be administered to a patient suffering from a PMND twice a day in combination with a Unit Form Component (b) manufactured
either in a tablet or capsule comprising a pharmaceutical carrier or vehicle for immediate release and pramipexole, in an amount (as pramipexole dihydrochloride monohydrate) of from 0.125 mg to 22.5 mg, to be administered to said patient twice a day;
or in a tablet or capsule or TDDS, for extended release, comprising a pharmaceutical carrier or vehicle and pramipexole, in an amount (as pramipexole dihydrochloride monohydrate) delivering from 0.325 mg to 45 mg once a day (in the case of a TDDS, over 24 hours).

In a similar domperidone/pramipexole/zonisamide combination, an Unit Form Component (ab) may be manufactured by using IR-formulated domperidone Component (a), in an amount (in domperidone base) of from 2 mg to 60 mg in a first layer or compartment and an IR-formulated pramipexole Component (b) amount (in in pramipexole dihydrochloride monohydrate) of from 0.125 mg to 22.5 mg, normally from 7.5 mg to 22.5 mg, from 7.5 to 12.5 mg or from 10.125 mg to 12.5 mg, in the second layer or compartment. This Unit Form may be administered to a patient suffering from a PMND twice a day in combination with a Unit Form Component (c), in an IR-formulation comprising zonisamide free acid an amount of from 25 mg to 300 mg, normally from 25 mg to 200 mg, to be administered twice a day, or in ER-formulation comprising zonisamide free acid in an amount of from 25 mg to 600 mg, normally from 100 mg to 400 mg, to be administered once a day.

In a domperidone/pramipexole/statin combination, a typical Unit Form Component (ac) may be manufactured by using a two-layer tablet or two-compartment capsule wherein domperidone Component (a), in an amount (in domperidone base) of from 2 mg to 60 mg, in admixture with a pharmaceutical carrier for immediate release, is in the first layer or compartment; and lovastatin Component (c), in an amount of from 5 mg to 40 mg, normally from 20 mg to 40 mg, in admixture with a pharmaceutical carrier for immediate release is in the second layer or compartment. This Unit Form may be administered to a patient suffering from a PMND twice a day in combination with a Unit Form Component (c) manufactured
either in a tablet or capsule comprising a pharmaceutical carrier or vehicle for immediate release and pramipexole, in an amount (in pramipexole dihydrochloride monohydrate) of from 0.125 mg to 22.5 mg, normally from 7.5 mg to 22.5 mg, from 7.5 mg to 12.5 mg, from more than 10 mg to 12.5 mg or from 10.125 mg, to 12.5 mg, to be administered to said patient twice a day;
or in a tablet or capsule or TDDS, for extended release, comprising a pharmaceutical carrier or vehicle and pramipexole, in an amount (as pramipexole dihydrochloride monohydrate) delivering from 0.325 mg to 45 mg, normally from 15 mg to 25 mg, from more than 20 mg to 25 mg or from 20.25 mg to 25 mg, to be administered to said patient once a day (in the case of a TDDS, over 24 hours).

In a similar typical domperidone/pramipexole/statin combination, an Unit Form Component (ab) may be manufactured by using IR-formulated domperidone, in an amount (in domperidone base) of from 2 mg to 60 mg in a first layer or compartment and an IR-formulated pramipexole amount (in in pramipexole dihydrochloride monohydrate) of from 0.125 mg to 22.5 mg or from 7.5 mg to 22.5 mg, normally from 7.5 mg to 12.5 mg, from more than 10 mg to 12.5 mg or from 10.125 mg to 12.5 mg, in the second layer or compartment. This Unit Form may be administered to a patient suffering from a PMND twice a day in combination with a Unit Form Component (c), in an IR-formulation comprising rosuvastatin calcium in an amount of from 2.5 mg to 40 mg, normally from 5 mg to 30 mg, in ER-formulation to be administered once a day.

### Kits

The present invention also provides a kit or package containing a medicament, a pharmaceutical combination, or a pharmaceutical composition as described herein, accompanied by instructions for use of the same in the treatment of a PMND in a patient in need thereof.

In one embodiment, a kit of the present invention is a kit comprising Unit Form Component (ac), wherein a domperidone and a statin are in admixture with a pharmaceutical carrier or vehicle; and instructions for use of the same for treatment of a PMND in a patient in need thereof, in combination with 6-propylamino-4,5,6,7-tetrahydro-1,3-benzothiazole-2-amine, preferably pramipexole.

In another embodiment, a kit of the present invention is a kit comprising pharmaceutical composition (a) comprising domperidone, a pharmaceutical composition (b) comprising 6-propylamino-4,5,6,7-tetrahydro-1,3-benzothiazole-2-amine, preferably pramipexole, and a pharmaceutical composition (c) comprising at least one of fluoxetine, zonisamide or a statin; and instructions for use of the same for treatment of a PMND in a patient in need thereof.

### EXAMPLE 1

A Phase I-II clinical study is conducted in parkinsonian subjects receiving oral doses of pramipexole or rosuvastatin, alone and in combination.

The objective of the study is to demonstrate that pramipexole and rosuvastatin, when administered together at their standard therapeutic doses, can safely normalize concentrations of synuclein species in peripheral blood exosomes.

To be enrolled in the study, male or female participants (40 to 89 years of age) are required to carry the diagnosis of Parkinson's disease or a related synucleinopathic disorder. All subjects sign an informed consent form indicating that they understand the purpose of and procedures required for the study and that they are willing to participate in the study and comply with all study procedures and restrictions. Key criteria for exclusion of a subject from enrollment in the study are as follows:
1. Any clinically relevant acute or chronic disease which could interfere with the subjects' safety during the trial, expose them to undue risk, or interfere with the study objectives.
2. History or presence of gastrointestinal, hepatic, or renal disease or other condition known to interfere with the absorption, distribution, metabolism or excretion of the study medications;
3. History of substance abuse, known drug addiction, or positive test for drugs of abuse or alcohol.
4. History of drug or other significant allergy.
5. Excessive daily consumption of xanthines containing drinks (i.e. >500 mg/day of caffeine).
6. Hospitalization or intake of an investigational drug within 30 days of study entry.

Following baseline clinical and laboratory evaluations, consenting individuals meeting accession criteria are first randomized to treatment with pramipexole titrated up to maximum tolerated dose (MTD), or up to a maximum dose of 5 mg/day, whichever came first. Patients are then maintained on pramipexole at their MTD or at 5 mg/day for 2 to 4 weeks. At the end of the maintenance period, venous blood for synuclein and drug assays is collected and patients are randomized to either rosuvastatin treatment (starting with 20 mg/day for approximately 2 weeks. If 20 mg/day is tolerated the dose of rosuvastatin is then to be increased to 40 mg/day (maximum recommended dose)) or placebo added on to pramipexole treatment. Patients are stably maintained on pramipexole and rosuvastatin (or placebo) treatment for 6 to 12 weeks. At the end of this combination treatment period venous blood for synuclein and drug assays is collected. Doses of both drugs are then tapered in accordance with current recommendations and patients are returned to their pre-admission regimen pending discharge from the study.

Drug safety-tolerability is monitored throughout the trial by means of standard clinical and laboratory tests. Weekly telephone interviews are generally conducted on those not scheduled for a clinic visit. A final safety check is performed approximately one month after withdrawal of all study medications.

Additionally, venous blood for synuclein and drug assays are collected during the study.

Results surprisingly show that the oral administration of a combination of pramipexole and rosuvastatin is associated with a tendency to normalize the characteristic alterations in synuclein and synuclein congener concentrations in exosomes collected from peripheral venous blood samples from patients who safely tolerated their therapeutic regimens.

In conclusion, the co-administration of standard approved doses of pramipexole and rosuvastatin provides clear evidence of a drug-combination-induced tendency to normalize synuclein processing indicative of a reduction in toxic species formation in the central nervous system of a type associated with a neuroprotective efficacy that clinically benefits patients suffering from Parkinson's disease or a related synucleinopathy.

### EXAMPLE 2

A Phase I-II clinical study is conducted in Parkinsonian subjects receiving oral high doses of pramipexole dihydrochloride monohydrate IR (“pramipexole”) with domperidone base (“domperidone”) IR with or without lovastatin IR in patients with moderately advanced PD.

The objective of the study, conducted as described in Example 1, is to demonstrate that high doses of pramipexole IR co-administered with recommended therapeutic doses of IR domperidone co-administered together with approved therapeutic doses of IR lovastatin, tend to safely normalize concentrations of synuclein species in brain-derived exosomes found in peripheral blood.

Results show that the oral administration of a combination of pramipexole and domperidone and lovastatin is associated with a tendency to rectify the characteristic alterations in synuclein and synuclein congener concentrations within brain-derived exosomes collected from peripheral venous blood samples from patients who safely tolerate their therapeutic regimens.

The co-administration of high doses of pramipexole in combination with standard doses of domperidone and lovastatin provides clear evidence of a drug-combination-induced tendency to rectify synuclein processing in the brain indicative of a reduction in toxic species formation in the central nervous system of a type associated with a neuroprotective efficacy that clinically benefits patients suffering from Parkinson's disease or a related synucleinopathy.

### EXAMPLE 3

The ability of domperidone to prevent the gastro-intestinal (GI) adverse effects (AEs) of pramipexole in humans is tested.

A Phase I study is conducted in subjects receiving a single oral dose of pramipexole dihydrochloride monohydrate (“pramipexole”) with or without a single oral dose of domperidone base (“domperidone”). The study is single center, single-blind study.

The objective of the study is to demonstrate that domperidone could safely attenuate the gastro-intestinal side effects of pramipexole given in doses equivalent or higher than those approved in the treatment of Parkinson's Disease or shown in clinical trials to be effective in the treatment of depression.

To be enrolled in the study, participants must meet the following inclusion/exclusion key criteria:

### Key Inclusion Criteria

1. Male and female subjects aged 20-45 years old both ages included.
2. Females of childbearing potential must agree to be abstinent or else use any two of the following medically acceptable forms of contraception from the Screening Period through 14 days after the study Exit Visit: condom with spermicidal jelly, diaphragm or cervical cap with spermicidal jelly, or intrauterine device (IUD). A female whose male partner has had a vasectomy must agree to use one additional form of medically acceptable contraception. Subjects must agree to practice the above birth control methods for 14 days after the final visit as a safety precaution.
3. Females of non-childbearing potential, defined as surgically sterile (status post-hysterectomy, bilateral oophorectomy, or bilateral tubal ligation) or post-menopausal for at least 12 months, do not require contraception during the study. The reason must be documented in the source documents.
4. Males with female partners of childbearing potential must agree to use a highly effective, medically acceptable form of contraception from the Screening Period through 14 days after the study Exit Visit. Males with female partners of childbearing potential who themselves are surgically sterile (status post vasectomy) must agree to use condoms with spermicide over the same period of time. Male subjects must agree to practice the above birth control methods for 14 days after the final visit as a safety precaution.
5. Subjects must be in good health as determined by their medical history including personal and family psychiatric history and results of physical examination, electrocardiogram (ECG), vital signs, and laboratory tests. A subject with a medical abnormality may be included only if the investigator or designee considers that the abnormality will not introduce significant additional risk to the subject's health or interfere with study objectives.
6. Subjects must be able to clearly and reliably communicate changes in their medical condition.
7. Subjects with a body mass index (BMI) between 19.0 and 32.0 kg/m² (both inclusive).
8. Subjects able to swallow multiple pills or capsules simultaneously.
9. Subjects must have signed an informed consent form indicating that they understand the purpose of and procedures required for the study and are willing to participate in the study and comply with the study procedures and restrictions.

### Key Exclusion Criteria:

The criteria for exclusion of a subject from enrollment in the study are as follows:
1. Any clinically relevant acute or chronic diseases which could interfere with the subjects' safety during the trial, expose them to undue risk, or interfere with the study objectives.
2. History or presence of gastrointestinal, hepatic, or renal disease or other condition known to interfere with the absorption, distribution, metabolism or excretion of the study drugs.
3. History of substance abuse, known drug addiction, or positive test for drugs of abuse or alcohol.
4. History of drug or other significant allergy.
5. Known hypersensitivity to pramipexole, or to domperidone or similar dopamine receptor antagonists.
6. History of and/or current QT interval prolongation, congenital long QT syndrome, electrolyte abnormalities (e.g., hypokalemia or hypomagnesemia), congestive heart failure, bradyarrhythmias or other medicinal products that lead to QT prolongation or 1st degree AV block at Screening, Day −1, or pre-dose, ≥450 QTcF for males and ≥470 QTcF for females.
7. Treatment with centrally active drugs or antiemetics, within 1 months of study entry.
8. Tobacco or nicotine users (except subjects who stopped using tobacco or nicotine 1 year or more before enrollment in the study).
9. Excessive daily consumption of xanthines containing drinks (i.e. >500 mg/day of caffeine).
10. Subjects unwilling to curtail prolonged intensive physical exercise during the study conduct (from the Screening visit until the last dose of study drug).
11. Positive test result for hepatitis B surface antigen, hepatitis C antibody.
12. Positive test result for HIV 1 or 2 serology.
13. Likely to need any medical or dental treatment during the study period.
14. Use of any prescription or over-the-counter medication within 14 days prior to admission on Day −1. In addition, any medications with central effects are prohibited for a period equal to 5 times the drug half-life prior to admission (Day −1), should this period be longer than 14 days.
15. Subjects unlikely to co-operate during the study, and/or be questionably compliant in the opinion of the investigator.
16. Subjects unable to be contacted in case of an emergency.
17. Intake of an investigational drug within 30 days of study entry.
18. Show evidence of suicidal ideation within the last 6 months as assessed by the C-SSRS (Columbia Suicide Severity Rating Scale) at Screening.

Following enrollment in the study, participants receive single increasing oral doses of pramipexole given once daily in the morning (Period 1 of the study). The starting dose of pramipexole was 0.5 mg and the dose is increased daily by 0.5 mg increments. Once a subject reaches his/her first intolerable dose (FID-1), upward dose escalation is discontinued. First intolerable dose (FID) is defined as:
one (1) episode of vomiting; or
Two (2) episodes of retching, or
One (1) episode of severe nausea (Grade 3; defined as nausea interfering with activities of daily living or inadequate oral caloric or fluid intake; tube feeding, total parenteral nutrition or hospitalization indicated) lasting more than 1 hour, or
Three (3) consecutive episodes at every 4 hour ratings of moderate nausea (Grade 2; defined as subjectively symptomatic, but not interfering with activities of daily living), or
One (1) episode of moderate diarrhea (Grade 2; defined as 4-6 stools more than at baseline).

When a subject reaches FID-1 on pramipexole alone, the subject is washed out for at least 5 days, and then enters Period 2 of the study during which the subject receives single daily oral doses of pramipexole starting at 0.5 mg and is titrated upward by 0.5 mg increments, together with oral domperidone (5 mg) until subjects again reach an intolerable dose defined as above. The FID on oral pramipexole plus oral domperidone is referred to as FID-2.

If a subject reaches FID-2 during Period 2 at the same or lower dose than FID-1, and providing the investigator judges there are no safety issues and the subject is consenting, the subject receives the same dose of pramipexole as the FID-2 dose together with a higher dose of oral domperidone (10 mg) on the next day and the protocol specifies that said subject should continue with the remainder of the dose titration with the higher dose of oral domperidone (10 mg) until they reach the intolerable dose (FID2+). All other provisions of the protocol remain unchanged. Assessments are the same as those planned for the dose escalation day.

On each study day, subjects are followed up for up to 8 hours following drug administration for AEs, vital signs, ECGs. In addition, a laboratory panel is taken at screening and at the end of the study.

Results showed that concomitant administration of domperidone with pramipexole prevents the occurrence of dose-limiting gastro-intestinal adverse events associated with high doses of pramipexole.

The Maximum Tolerated Dose (MTD) of pramipexole during Period 2 is higher than MTD during Period 1.

Taken together, results show that the co-administration of domperidone with pramipexole attenuates dose-limiting gastro-intestinal adverse effects reported with pramipexole alone, thus showing that domperidone enables the administration to a human being of pramipexole in doses otherwise non-tolerated when administering pramipexole alone.

### EXAMPLE 4

A Phase I-II clinical study is conducted in Parkinsonian subjects receiving oral high doses of pramipexole dihydrochloride monohydrate IR (“pramipexole”) with domperidone base (“domperidone”) IR with or without fluoxetine IR in patients with moderately advanced PD.

The objective of the study, conducted as described in Example 3, is to demonstrate that high doses of pramipexole IR co-administered with recommended therapeutic doses of IR domperidone co-administered together with approved therapeutic doses of IR fluoxetine, tend to safely normalize concentrations of synuclein species in brain-derived exosomes found in peripheral blood.

Results show that the oral administration of a combination of pramipexole and domperidone and fluoxetine is associated with a tendency to normalize the characteristic alterations in synuclein and synuclein congener concentrations within brain-derived exosomes collected from peripheral venous blood samples from patients who safely tolerate their therapeutic regimens.

The co-administration of high doses of pramipexole in combination with standard doses of domperidone and fluoxetine provides clear evidence of a drug-combination-induced tendency to normalize synuclein processing in the brain indicative of a reduction in toxic species formation in the central nervous system of a type associated with a neuroprotective efficacy that clinically benefits patients suffering from Parkinson's disease or a related synucleinopathy.

### EXAMPLE 5

A Phase I-II clinical study is conducted in Parkinsonian subjects receiving oral high doses of pramipexole dihydrochloride monohydrate IR (“pramipexole”) with domperidone base (“domperidone”) IR with or without zonisamide IR in patients with moderately advanced PD. The objective of the study, conducted as described in Example 3, is to demonstrate that high doses of pramipexole IR co-administered with recommended therapeutic doses of IR domperidone co-administered together with approved therapeutic doses of IR zonisamide, tend to safely normalize concentrations of synuclein species in brain-derived exosomes found in peripheral blood.

Results show that the oral administration of a combination of pramipexole and domperidone and zonisamide is associated with a tendency to normalize the characteristic alterations in synuclein and synuclein congener concentrations within brain-derived exosomes collected from peripheral venous blood samples from patients who safely tolerate their therapeutic regimens.

The co-administration of high doses of pramipexole in combination with standard doses of domperidone and zonisamide provides clear evidence of a drug-combination-induced tendency to normalize synuclein processing in the brain indicative of a reduction in toxic species formation in the central nervous system of a type associated with a neuroprotective efficacy that clinically benefits patients 5 suffering from Parkinson's disease or a related synucleinopathy.

### REFERENCES

Al-Mansoori et al. 2013: Al-Mansoori K M, Hasan M Y, Al-Hayani A, El-Agnaf M, “The role of α-synuclein in neurodegenerative diseases: from molecular pathways in disease to therapeutic approaches”; Curr. Alzheimer Res. 2013 July; 10(6): 559-568.
Arawaka S et al. 2014: Arawaka S, Fukushima S, Sato H, Sasaki A, Koga K, Koyama S, Kato T; “Zonisamide Attenuates α-Synuclein Neurotoxicity by an Aggregation Independent Mechanism in a Rat Model of Familial Parkinson's Disease”; PLOS One. 2014; 9(2): e89076.
Bar-On et al 2008: Bar-On P, Crews L, Koob A O, Mizuno H, Adame A, Spencer B, Masliah E. “Statins reduce neuronal alpha-synuclein aggregation in in vitro models of Parkinson's disease”. J Neurochem. 2008 June; 105(5):1656-67. doi: 10.1111/j.1471-4159.2008.05254.x. Epub 2008 Jan. 28.
Braak et al. 2003: Braak H, Del Tredici K, Rüb U, de Vos R A, Jansen Steur E N, Braak E. “Staging of brain pathology related to sporadic Parkinson's disease”. Neurobiol Aging. 2003 March-April; 24(2):197-211.
Brogden et al 1982: Brogden R N, Carmine A A, Heel R C, Speight T M, Avery G S. “Domperidone. A Review of its Pharmacological Activity, Pharmacokinetics and Therapeutic Efficacy in the Symptomatic Treatment of Chronic Dyspepsia and as an Antiemetic”. Drugs 1982 (November) 24(5):360-400.
Bruni et al 2013: Bruni G, Maietta M, Scotti F, Maggi L, Bini M, Ferrari S, Capsoni D, Boiocchi M, Berbenni V, Milanese C, Girella A, Marini A. “Structure and properties of domperidone and its succinate salt” Acta Crystallogr B Struct Sci Cryst Eng Mater. 2013 August; 69(Pt 4):362-70. doi: 10.1107/S2052519213016989. Epub 2013 Jul. 18.
Bymaster et al. 2002: Bymaster F, Zhang W, Carter P, Shaw J, Chemet E; Phebus L, Wong D, Perry K; “Fluoxetine, but not other selective serotonin uptake inhibitors, increases norepinephrine and dopamine extracellular levels in prefrontal cortex via serotonin type 2C antagonism”. Psychopharmacology 2002 160 (4): 353-61.
Carrol G B and Wyse R K H. 2017; Carroll C B, Wyse R K H. “Simvastatin as a Potential Disease-Modifying Therapy for Patients with Parkinson's Disease: Rationale for Clinical Trial, and Current Progress”. J Parkinsons Dis. 2017; 7(4):545-568.
Carrol J A et al. 2017: Carroll J A; Race B; Phillips K; Striebel J F; Chesebro B. “Statins are ineffective at reducing neuroinflammation or prolonging survival in scrapie-infected mice”. J Gen Virol; 98(8):2190-2199, 2017 August 1465-2099.
Castillo-Carranza et al. 2018: Castillo-Carranza D L, Marcos J. Guerrero-Muñoz M J, Sengupta U, Gerson J E, Kayed R; “α-Synuclein Oligomers Induce a Unique Toxic Tau Strain”, Biol. Psychiatry, pii: S0006-3223(18)30034-9 (January 2018).
Carrol G B and Wyse R K H. 2017; Carroll C B, Wyse R K H. “Simvastatin as a Potential Disease-Modifying Therapy for Patients with Parkinson's Disease: Rationale for Clinical Trial, and Current Progress”. J Parkinsons Dis. 2017; 7(4):545-568.
Carrol J A et al. 2017: Carroll J A; Race B; Phillips K; Striebel J F; Chesebro B. “Statins are ineffective at reducing neuroinflammation or prolonging survival in scrapie-infected mice”. J Gen Virol; 98(8):2190-2199, 2017 August 1465-2099.
Chataway et al 2014: Chataway J, Schuerer N, Alsanousi A, Chan D, MacManus D, Hunter K, Anderson V, Bangham C R, Clegg S, Nielsen C, Fox N C, Wilkie D, Nicholas J M, Calder V L, Greenwood J, Frost C, Nicholas R. “Effect of high-dose simvastatin on brain atrophy and disability in secondary progressive multiple sclerosis (MS-STAT): a randomised, placebo-controlled, phase 2 trial”. Lancet. 2014 Jun. 28; 383(9936):2213-21. doi: 10.1016/S0140-6736(13)62242-4. Epub 2014 Mar. 19.
Chen et al. 2016: Chen M, Weiwei Yang W, Li Xin, X, Li Xuran, Wang P, Feng Yue F, Yang H, Chan P, and Yu S; “Age- and brain region-dependent α-synuclein oligomerization is attributed to alterations in intrinsic enzymes regulating α-synuclein phosphorylation in aging monkey brains”; Oncotarget. 2016 Feb. 23; 7(8): 8466-8480.
Choi and Gandhi 2018: Choi M L, Gandhi S. “Crucial role of protein oligomerization in the pathogenesis of Alzheimer's and Parkinson's diseases”. FEBS J. 2018 Jun. 20).
Choksi et al. 2014: Choksi D K, Roy B, Chatterjee S, Yusuff T, Bakhoum M. F, Sengupta U, Ambegaokar S, Kayed R, Jackson G R. “TDP-43 phosphorylation by casein kinase Iepsilon promotes oligomerization and enhances toxicity in vivo”. Hum Mol Genet 2014, 23, 1025-1035.
Chow et al. 2010: Chow V W, Savonenko A V, Melnikova T, Kim H, Price D L, Li T, Wong P C. “Modeling an anti-amyloid combination therapy for Alzheimer's disease”. Sci Transl Med. 2010 Jan. 6; 2(13):13ral. PubMed. 8 Jan. 2010.
Chung et al. 2011: Chung Y C, Kim S R, Park J Y, Chung E S, Park K W, Yoon S H, Ko H W, Kim Y S, Jin B K. “Fluoxetine prevents MPTP-induced loss of dopaminergic neurons by inhibiting microglial activation”. Neuropharmacology. 2011 May; 60(6):963-74, doi: 10.1016/j.neuropharm.2011.01.043. Epub 2011 Feb. 1.
Cline et al. 2018: Cline E N, Bicca M A, Viola K L, Klein W L “The Amyloid-β Oligomer Hypothesis: Beginning of the Third Decade”. J Alzheimer Dis. 2018; 64(s1): S567-S610.
Collins et al. 2016: Collins R, Reith C, Emberson J, Armitage J, Baigent C, Blackwell L, Blumenthal R, Danesh J, Davey Smith G, DeMets D, Evans S, Law M, MacMahon S, Martin S, Neal B, Poulter N, Preiss D, Ridker P, Roberts I, Rodgers A, Sandercock P, Schulz K, Sever P, Simes J, Smeeth L, Wald N, Yusuf S, Peto R. “Interpretation of the evidence for the efficacy and safety of statin therapy”. Lancet. 2016 Nov. 19; 388(10059):2532-2561.
Connolly and Lang 2014: Connolly B S, Lang A E, “Pharmacological treatment of Parkinson disease: a review”. JAMA. 2014 Apr. 23-30; 311(16):1670-83).
Corrigan et al. 2000: Corrigan M H, Denahan A Q, Wright C E, Ragual R J, Evans D L; Corrigan M H, Denahan A Q, Wright C E, Ragual R J, Evans D; “Comparison of pramipexole, fluoxetine, and placebo in patients with major depression”; Depress Anxiety. 2000; 11(2):58-65.
Costa et al. 2010: Costa C, Tozzi A, Luchetti E, Siliquini S, Belcastro V, Tantucci M, Picconi B, Ientile R, Calabresi P, Pisani F. “Electrophysiological actions of zonisamide on striatal neurons: Selective neuroprotection against complex I mitochondrial dysfunction”; Exp Neurol. 2010 January; 221(1):217-24.
Erro Aguirre et al. 2015: Erro Aguirre M E, Zelaya M V, Sánchez Ruiz de Gordoa J, Tuñón M T, Lanciego J L; “Midbrain catecholaminergic neurons co-express α-synuclein and tau in progressive supranuclear palsy”; Front Neuroanat. 2015 Mar. 11; 9:25. doi: 10.3389/fnana.2015.00025. eCollection 2015 (March 2105).
Fang et al. 2014: Fang Y S, Tsai K J, Chang Y J, Kao P, Woods R, Kuo, P H, Wu C C, Liao J Y, Chou S C, Lin V, Jin L W, Yuan H S, Cheng I H, Tu P H, Chen Y R. “Full-length TDP-43 forms toxic amyloid oligomers that are present in frontotemporal lobar dementia-TDP patients”. Nat Commun 2014, 5, 4824.
Gandy et al. 2010: Gandy S, Simon A J, Steele J W, Lublin, A L, Lah, J J, Walker L C, Levey A I, Krafft G A, Levy E, Checler F, Glabe C, Bilker W., Abel T. Schmeidler J., Ehrlich M E. “Days-to-criterion as an indicator of toxicity associated with human Alzheimer amyloid-β oligomers”. Ann Neurol. 2010, 68, 220-230.
Gerson and Kayed 2013: Gerson J E, Kayed R. “Formation and propagation of tau oligomeric seeds”. Front Neurol 2013, 4, 93.
Glabe 2006: Glabe C G, “Common mechanisms of amyloid oligomer pathogenesis in degenerative disease”. Neurobiol. Aging 2006 27, 570-575.
Hoffner and Djian 2014: Hoffner G., Djian P. “Monomeric, oligomeric and polymeric proteins in huntington disease and other diseases of polyglutamine expansion”. Brain Sci. 2014 Mar. 3; 4(1):91-122.
Inden et al. 2009: Inden M, Kitamura Y, Tamaki A, Yanagida T, Shibaike T, Yamamoto A, Takata K, Yasui H, Taira T, Ariga H, Taniguchi T; “Neuroprotective effect of the antiparkinsonian drug pramipexole against nigrostriatal dopaminergic degeneration in rotenone-treated mice.”; Neurochem Int. 2009 December; 55(8):760-7.
Jellinger 2008a: Jellinger K A, “A critical reappraisal of current staging of Lewy-related pathology in human brain”; Acta Neuropathol. 2008 July; 116(1): 1-16.
Jellinger 2008b: Jellinger K A, “Neuropathological aspects of Alzheimer disease, Parkinson disease and frontotemporal dementia”; Neurodegener. Dis. 2008; 5(3-4): 118-121.
Kakimura et al. 2001: Kakimura J, Kitamura Y, Takata K, Kohno Y, Nomura Y, Taniguchi T; “Release and aggregation of cytochrome c and alpha-synuclein are inhibited by the antiparkinsonian drugs, talipexole and pramipexole”; Eur J Pharmacol. 2001 Apr. 6; 417(1-2):59-67.
Kang al. 2017: Kang S Y, Lee S B, Kim H J, Kim H T, Yang H O, Jang W; “Autophagic modulation by rosuvastatin prevents rotenone-induced neurotoxicity in an in vitro model of Parkinson's disease”; Neurosci Lett. 2017 Mar. 6; 642:20-26).
Kataoka and Ueno 2014: Kataoka H Ueno S. “Auditory Musical Hallucinations Associated With Extended-Release Pramipexole in an Elderly Patient With Parkinson's Disease”. Medicine: December 2014-93, 27 e251.
Keowkase et al. 2010: Keowkase R, Aboukhatwa M, Luo Y. “Fluoxetine protects against amyloid-beta toxicity, in part via daf-16 mediated cell signaling pathway, in Caenorhabditis elegans”. Neuropharmacology. 2010 September-October; 59(4-5):358-65.
Kim et al. 2004: Kim S, Seo J H, Suh Y H, “Alpha-synuclein, Parkinson's disease, and Alzheimer's disease”; Parkinsonism Relat. Disord. 2004 May; 10 Suppl. 1: S9-13.
Kumar et al. 2012: Kumar A, Sharma N, Gupta A, Kalonia H, Mishra J. “Neuroprotective potential of atorvastatin and simvastatin (HMG-CoA reductase inhibitors) against 6-hydroxydopamine (6-OHDA) induced Parkinson-like symptoms”. Brain Res. 2012 Aug. 30; 1471:13-22.
Latha et al. 2012: Latha S, Selvamani P, Lakshmana Prabu S, Santhosh Kumar P, Pal T K. “Formulation and Evaluation of Matrix Diffusion Controlled Transdermal Patches of Domperidone hydrochloride”. Ars Pharm. 2011; 52(1): 31-36.
Lin et al. 2015: Lin C M, Lin Y T, Lin R D, Huang W J, Lee M H “Neurocytoprotective Effects of Aliphatic Hydroxamates from Lovastatin, a Secondary Metabolite from Monascus-Fermented Red Mold Rice, in 6-Hydroxydopamine (6-OHDA)-Treated Nerve Growth Factor (NGF)-Differentiated PC12 Cells”. ACS Chem Neurosci. 2015 May 20; 6(5):716-24).
Luo et al. 2016: Luo H T, Zhang J P, Miao F; “Effects of pramipexole treatment on the α-synuclein content in serum exosomes of Parkinson's disease patients”; ExpTher Med. 2016 September; 12(3):1373-1376.
Madishetti et al 2010: Madishetti S K, Palem C R, Gannu R, Thatipamula R P, Panakanti P K, and Yamsani M R; “Development of domperidone bilayered matrix type transdermal patches: physicochemical, in vitro and ex vivo characterization”; Daru 2010; 18(3): 221-229.
Marques and Outeiro 2012: Marques O, Outeiro T F; “Alpha-synuclein: from secretion to dysfunction and death”; Cell Death Dis. 2012 Jul. 19; 3:e350. doi: 10.1038/cddis.2012.94.
Mitchell Sontag et al. 2012: Mitchel Sontag E, Sontag C, Cumming B J, Muchowski P J (+4); “Detection of Mutant Huntingtin Aggregation Conformers and Modulation of SDS-Soluble Fibrillar Oligomers by Small Molecules”; Journal of Huntington's Disease (June 2012).
Nilson et al. 2017: Nilson A N, English K C, Gerson J E, Barton Whittle T, Nicolas Crain C, Xue J, Sengupta U, Castillo-Carranza D L, Zhang W, Gupta P, Kayed R, Tau Oligomers Associate with Inflammation in the Brain and Retina of Tauopathy Mice and in Neurodegenerative Diseases, J. Alzheimers Dis., 55(3): 1083-1099 (2017).
Ono et al. 2013: Ono K, Takasaki J, Takahashi R, Ikeda T, Yamada M; “Effects of antiparkinsonian agents on β-amyloid and α-synuclein oligomer formation in vitro”; JNeurosci Res; 2013 October; 91(10):1371-81).
O'Regan et al. 2017: O'Regan G, DeSouza R M, Balestrino R, “Glucocerebrosidase Mutations in Parkinson Disease”. J Parkinson's Dis 7 (2017) 411-422—DOI 10.3233/JPD-171092.
Orr J D 2008: Orr J; “Statins in the spectrum of neurologic disease” Curr Atheroscler Rep. 2008 February; 10(1):11-8.
Petersen et al. 2013: Petersen R C, Aisen P, Boeve B F, Geda Y E, Ivnik R J, Knopman D S, Mielke M, Pankratz V S, Roberts R, Rocca W A, Weigand S, Weiner M, Wiste H, Jack C R. “Criteria for mild cognitive impairment due to Alzheimer's disease in the community”. Ann. Neurol. 2013, 74, 199-208.
Poewe et al. 2017: Poewe W, Seppi K, Tanner C M, Halliday G M, Brundin P, Volkmann J, Schrag A E, Lang A E. “Parkinson disease”. Nat Rev Dis Primers. 2017 Mar. 23; 3:17013.
Prabhu et al 2011: Prabhu P, Shah S, Gundad S, “Formulation development and investigation of domperidone transdermal patches”. Int J Pharm Investig. 2011 October-December; 1(4):240-246.
Prusiner et al. 2015: Prusiner S B¹, Woerman A L², Mordes D A³, WaTDDS J C⁴, Rampersaud R², Berry D B², Patel 5², Oehler A⁵, Lowe J K⁶, Kravitz S N⁶, Geschwind D H⁷, Glidden D V⁸, Halliday G M⁹, Middleton L T¹⁰, Gentleman S M¹¹, Grinberg L T¹², Giles K⁴, “Evidence for α-synuclein prions causing multiple system atrophy in humans with parkinsonism” ProcNatlAcadSci USA; 2015, Sep. 22; 112(38):E5308-17.
Reddymasu et al. 2007: Reddymasu S C, Soykan I, Richard W McCallum R W. “Domperidone: Review of Pharmacology and Clinical Applications in Gastroenterology”. Am J Gastroenterology. 2007, 102, 2036-2045.
Rogóz and Skuza 2006: Rogóz Z and Skuza G. “Mechanism of synergistic action following co-treatment with pramipexole and fluoxetine or sertraline in the forced swimming test in rats”, Pharmacol Rep 2006 58(4):493-500.
Reich and Grill 2009: Reich S G, Grill S E. “Corticobasal degeneration” Curr Treat Options Neurol. 2009 May; 11(3):179-85.
Roy and Pahan 2011: Roy A, Pahan K. “Prospects of statins in Parkinson disease”. Neuroscientist. 2011 June; 17(3):244-55.
Samuels et al. 2007: Samuels E R, Hou R H, Langley R W, Szabadi E, Bradshaw C M. “Comparison of pramipexole with and without domperidone co-administration on alertness, autonomic, and endocrine functions in healthy volunteers”. Br J Clin Pharmacol. 2007 November; 64(5):591-602.
Sangwan et al. 2017: Sangwan S, Zhao A, Adams K L, Jayson C K, Sawaya M R, Guenther E L, Pan A C, Ngo J, Moore D M, Soriaga A B, Do T D, Goldschmidt L, Nelson R, Bowers M T, Koehler C M, Shaw D E, Novitch B G, Eisenberg D S. “Atomic structure of a toxic, oligomeric segment of SOD1 linked to amyotrophic lateral sclerosis (ALS)”. Proc Natl Acad Sci USA. 2017 Aug. 15; 114(33):8770-8775).
Sano et al 2015: Sano H, Murata M, Nambu A; “Zonisamide reduces nigrostriatal dopaminergic neurodegeneration in a mouse genetic model of Parkinson's disease”; J Neurochem. 2015 July; 134(2):371-81.
Schapira et al. 2013: Schapira A H, McDermott M P, Barone P, Comella C L, Albrecht S, Hsu H H, Massey D H, Mizuno Y, Poewe W, Rascol O, Marek K. “Pramipexole in patients with early Parkinson's disease (PROUD): a randomised delayed-start trial”; Lancet Neurol. 2013 August; 12(8):747-55).
Schneider and Mierau 1987: Schneider C S, Mierau J “Dopamine autoreceptor agonists: resolution and pharmacological activity of 2,6-diaminotetrahydrobenzothiazole and an aminothiazole analogue of apomorphine”; J. Med Chem. 1987 March; 30(3):494-8.
Sengupta et al. 2016: Sengupta U, Nilson A, Kayed R. “The Role of Amyloid-β Oligomers in Toxicity, Propagation, and Immunotherapy”. EBioMedicine 6, Apr. 2016. DOI: 10.1016/j.ebiom.2016.03.035.
Sengupta et al. 2017: Sengupta U, Portelius E, Hansson O, Farmer K, Castillo-Carranza D, Woltjer R, Zetterberg H, Galasko D, Blennow K, Kayed; “Tau oligomers in cerebrospinal fluid in Alzheimer's disease”. Ann Clin Transl Neurol. 2017 April; 4(4): 226-235.
Shi et al. 2014: Shi M, Liu C, Cook T J, Bullock K M, Zhao Y, Ginghina C, Li Y, Aro P, Dator R, He C, Hipp M J, Zabetian C P, Peskind E R, Hu S C, Quinn J F, Galasko D R, Banks W A, Zhang J; “Plasma exosomal α-synuclein is likely CNS-derived and increased in Parkinson's disease”; ActaNeuropathol. 2014 November; 128(5):639-50. doi: 10.1007/s00401-014-1314-y. Epub 2014 Jul. 6.
Shirisha et al. 2017: Shirisha S, Joshi G, Sahoo G K, Rao Y M; “Preparation and Evaluation of Matrix Type Transdermal Patches of Domperidone Maleate: in vitro and ex vivo Characterization”; Indian J of Pharmaceutical Education and Research. 2017; 51(4):517-24.
Shults et al. 2005: Shults C W, Rockenstein E, Crews L, Adame A, Mante M, Larrea G, Hashimoto M, Song D, Iwatsubo T, Tsuboi K, Masliah E.; “Neurological and neurodegenerative alterations in a transgenic mouse model expressing human alpha-synuclein under oligodendrocyte promoter: implications for multiple system atrophy”; J Neurosci 2005, Nov. 16; 25(46):10689-99.
Soria et al. 2017: Soria F N, Engeln M, Martinez-Vicente M, Glangetas C, Lopez-Gonzales J, Dovero S, Dehay B, Normand E, Vila M, Lopez-Gonzales M J, Favereaux A, Georges F, Lo Bianco C, Bezard E, Femagut; “Glucocerebrosidase deficiency in dopaminergic neurons induces microglial activation without neurodegeneration”; Hum Mol Genet 2017 July; 26(14):2603-2615.
Stuendl et al. 2016: Stuendl A, Kunadt M, Kruse N, Bartels C, Moebius W, Danzer K M, Mollenhauer B, Schneider A; “Induction of alpha-synuclein in aggregate formation by CSF exosomes from patients with Parkinson's disease and dementia with Lewy bodies” Brain 2016, 139; 481-494
Sweeney et al. 2017: Sweeney P, Park H, Baumann M, Dunlop J, Frydman J, Kopito R, McCampbell A, Leblanc G, Venkateswaran A, Nurmi A, Hodgson R. “Protein misfolding in neurodegenerative diseases: implications and strategies”. Transl Neurodegener. 2017 Mar. 13; 6:6. doi: 10.1186/s40035-017-0077-5. eCollection 2017.
Ubhi et al. 2012: Ubhi K, Inglis C, Mante M, Patrick C, Adame A, Spencer B, Rockenstein E, May V, Winkler J, Masliah; “Fluoxetine ameliorates behavioral and neuropathological deficits in a transgenic model mouse of α-synucleinopathy”; E. Exp Neurol. 2012 April; 234(2):405-16.
Visanji et al. 2016: Visanji N P, Brotchie J M, Kalia L V, Koprich J B, Tandon A, WaTDDS J C, Lang A E; “α-Synuclein-Based Animal Models of Parkinson's Disease: Challenges and Opportunities in a New Era”; Trends Neurosci. 2016 November; 39(11):750-762.
Willey and Elkind 2010: Willey J Z, Elkind M S V. “HMG-CoA Reductase Inhibitors in the Treatment of Diseases of the Central Nervous System”. Arch Neurol 2010 September; 67(9):1062-7.
Xu et al. 2013: Xu Y Q, Long L, Yan J Q, Wei L, Pan M Q, Gao H M, Zhou P, Liu M, Zhu C S, Tang B S, Wang Q. “Simvastatin induces neuroprotection in 6-OHDA-lesioned PC12 via the PI3K/AKT/caspase 3 pathway and anti-inflammatory responses”. CNS Neurosci Ther. 2013 March; 19(3):170-7.
Yan et al. 2015: Yan J Q, Sun J C, Zhai M M, Cheng L N, Bai X L, Feng C L “Lovastatin induces neuroprotection by inhibiting inflammatory cytokines in 6-hydroxydopamine treated microglia cells”. Int J Clin Exp Med. 2015 Jun. 15; 8(6):9030-7.
Yokoyama H et al. 2010: Yokoyama H, Yano R, Kuroiwa H, Tsukada T, Uchida H, Kato H, Kasahara J, Araki T; “Therapeutic effect of a novel anti-Parkinsonian agent zonisamide against MPTP (1-methyl-4-phenyl-1,2,3,6-tetrahydropyridine) neurotoxicity in mice”; Metab Brain Dis 2010 June; 25(2):135-43.

1. Domperidone or a pharmaceutically acceptable salt or solvate thereof, for use for the treatment of a protein misfolding neurodegenerative disease (PMND) in a patient or use of domperidone or a pharmaceutically acceptable salt or solvate thereof for the preparation of a medicament, in combination with 6-propylamino-4,5,6,7-tetrahydro-1,3-benzothiazole-2-amine or pharmaceutically acceptable salt or solvate thereof, and at least one synergistic agent selected from the group consisting of fluoxetine and pharmaceutically acceptable salts and solvates thereof, zonisamide, and statins.

2. Domperidone or a pharmaceutically acceptable salt or solvate thereof, for use or use of domperidone or a pharmaceutically acceptable salt or solvate thereof for the preparation of a medicament according to claim 1, wherein, in said combination, said domperidone or pharmaceutically acceptable salts or solvates thereof, said 6-propylamino-4,5,6,7-tetrahydro-1,3-benzothiazole-2-amine or pharmaceutically acceptable salts or solvates thereof, and said at least one synergistic agent are each formulated in a pharmaceutical composition, each in admixture with a pharmaceutical carrier or vehicle.

3. Domperidone or a pharmaceutically acceptable salts or solvate thereof, for use or use of domperidone or a pharmaceutically acceptable salts or solvate thereof for the preparation of a medicament according to claim 1, wherein, in said combination, said domperidone or pharmaceutically acceptable salts or solvates thereof and said at least one synergistic agent are formulated in a pharmaceutical composition comprising a parmaceutical carrier or vehicle and a fixed-dose combination of said domperidone or pharmaceutically acceptable salts or solvates thereof and said at least one synergistic agent.

4. Domperidone or a pharmaceutically acceptable salts or solvate thereof, for use or use of domperidone or a pharmaceutically acceptable salts or solvate thereof for the preparation of a medicament according to claim 3, wherein, in said pharmaceutical composition, said domperidone or pharmaceutically acceptable salt or solvate thereof is present in an amount equivalent to from 2 mg to 120 mg of domperidone base, and said at least one synergistic agent is selected from the group consisting of fluoxetine and pharmaceutically acceptable salts and solvates thereof, in an amount equivalent to from 2 mg to 90 mg of fluoxetine base; zonisamide and pharmaceutically acceptable salts and solvates thereof, in an amount equivalent to from 25 mg to 600 mg of zonisamide free acid; and a statin, in an amount of from 0.5 mg to 80 mg.

5. Domperidone or a pharmaceutically acceptable salts or solvate thereof, for use or use of domperidone or a pharmaceutically acceptable salts or solvate thereof for the preparation of a medicament according to claim 4, wherein said statin in said composition is is selected from the group consisting of lovastatin, in an amount per unit form of from 5 mg to 60 mg; and rosuvastatin calcium, in an amount per unit form of from 2.5 mg to 40 mg.

6. Domperidone or a pharmaceutically acceptable salts or solvate thereof, for use or use of domperidone or a pharmaceutically acceptable salts or solvate thereof for the preparation of a medicament according to claim 4, wherein said composition further comprises 6-propylamino-4,5,6,7-tetrahydro-1,3-benzothiazole-2-amine in an amount equivalent to from 0.125 mg to 3000 mg of pramipexole dihydrochloride monohydrate, said amount including a (S)-enantiomer amount equivalent to from 0.125 mg to 45 mg of pramipexole dihydrochloride monohydrate.

7. Domperidone or a pharmaceutically acceptable salts or solvate thereof, for use or use of domperidone or pharmaceutically acceptable salts or solvate thereof for the preparation of a medicament according to claim 6, wherein, said pharmaceutical composition, said 6-propylamino-4,5,6,7-tetrahydro-1,3-benzothiazole-2-amine is selected from the group consisting of pramipexole and pharmaceutically acceptable salts and solvates thereof, in an amount equivalent to from 0.125 mg to 45 mg of pramipexole dihydrochloride monohydrate.

8. Domperidone or a pharmaceutically acceptable salts or solvate thereof, for use or use of domperidone or a pharmaceutically acceptable salts or solvate thereof for the preparation of a medicament according to claim 7, wherein, in said pharmaceutical composition, said pramipexole or pharmaceutically acceptable salts or solvates thereof is present in an amount-range in pramipexole dihydrochloride monohydrate selected from the group consisting of from 7.5 mg to 25 mg, from from 15 mg to 25 mg, from more than 20 mg to 25 mg and from 20.25 mg to 25 mg.

9. Domperidone or a pharmaceutically acceptable salts or solvate thereof, for use or use of domperidone or a pharmaceutically acceptable salts or solvate thereof for the preparation of a medicament according to claim 1, wherein said PMND is selected from the group consisting of Alzheimer's disease, Parkinson's disease, Lewy body dementia, dementia with Lewy bodies, the Lewy body variant of Alzheimer's disease, multiple system atrophy, neurodegeneration with brain iron accumulation, Parkinsonian disorders associated with glucocerebrosidase mutations, Huntington's Disease, corticobasal degeneration, frontotemporal dementia with parkinsonism-linked to chromosome 17, Pick's Disease, Multiple Tauopathies, Amyotrophic Lateral Sclerosis, Spongiform encephalopathies, and Familial Amyloidotic Polyneuropathy.

10. A pharmaceutical composition comprising:
(a) domperidone or a pharmaceutically acceptable salt or solvate thereof;
(b) a 6-propylamino-4,5,6,7-tetrahydro-1,3-benzothiazole-2-amine or a pharmaceutically acceptable salt or solvate thereof; and
(c) at least one synergistic agent selected from the group consisting of fluoxetine and pharmaceutically acceptable salts and solvates thereof, zonisamide and pharmaceutically acceptable salts and solvates thereof, and statins;
in admixture with a pharmaceutical carrier or vehicle.

11. The pharmaceutical composition of claim 10, wherein said pharmaceutical composition is in a dosage unit form wherein
(a) said domperidone is in an amount per unit form equivalent to from 2 mg to 120 mg of domperidone base;
(b) said 6-propylamino-4,5,6,7-tetrahydro-1,3-benzothiazole-2-amine is selected from the group consisting of
(i) the racemate or a pharmaceutically acceptable salt thereof, in an amount per unit form equivalent to from 0.25 mg to 90 mg of pramipexole dihydrochloride monohydrate,
(ii) pramipexole or a pharmaceutically acceptable salt thereof, in an amount equivalent to from 0.125 mg to 45 mg of pramipexole dihydrochloride monohydrate, and
(iii) a (R)/(S)-mixture, in an amount per unit form of from 50 mg to 3000 mg, inclusive of a (S)-enantiomer amount per unit form equivalent to from 0.125 mg to 45 mg of pramipexole dihydrochloride monohydrate; and
(c) said at least one synergistic agent is selected from the group consisting of fluoxetine, in an amount per unit equivalent to form of from 2 mg to 90 mg of fluoxetine base; zonisamide, in an amount per unit form equivalent to from 25 mg to 600 mg of zonisamide free acid; and a statin, in an amount of from 0.5 mg to 80 mg.

12. The pharmaceutical composition of claim 12, wherein said 6-propylamino-4,5,6,7-tetrahydro-1,3-benzothiazole-2-amine is pramipexole or a pharmaceutically acceptable salt thereof, in an amount per unit form selected from the group consisting of
an amount per unit form equivalent to from more than 4.5 mg to 45 mg of pramipexole dihydrochloride monohydrate,
an amount per unit form equivalent to from more than 6 mg to 45 mg of pramipexole dihydrochloride monohydrate,
an amount per unit form equivalent to from 6.5 mg to 45 mg of pramipexole dihydrochloride monohydrate,
an amount per unit form equivalent to from 7.5 mg to 25 mg of pramipexole dihydrochloride monohydrate,
an amount per unit form equivalent to from 15 mg to 25 mg of pramipexole dihydrochloride monohydrate, and
an amount per unit form equivalent to from 20.25 mg to 25 mg of pramipexole dihydrochloride monohydrate.

13. The pharmaceutical composition of claim 10, wherein said pharmaceutical composition is in a dosage unit form wherein
(a) said domperidone is selected from the group consisting of domperidone base, domperidone hydrochloride, domperidone maleate and domperidone succinate (1:1), in an amount per unit form equivalent to from 2 mg to 120 mg of domperidone base;
(b) said 6-propylamino-4,5,6,7-tetrahydro-1,3-benzothiazole-2-amine is selected from the group consisting of pramipexole base and pramipexole dihydrochloride monohydrate, in an amount per unit form equivalent to from 0.125 mg to 45 mg of pramipexole dihydrochloride monohydrate; and
(c) said at least one synergistic agent is selected from the group consisting of lovastatin, in an amount per unit form of from 5 mg to 60 mg; and rosuvastatin calcium, in an amount per unit form of from 2.5 mg to 40 mg.

14. A kit comprising the pharmaceutical composition according to claim 10, and instructions for use for the treatment of a PMND in a patient.

15. A method for treating a protein misfolding neurodegenerative disease (“PMND”) in a patient, which comprises administering to said patient in need of said treatment domperidone, in combination with 6-propylamino-4,5,6,7-tetrahydro-1,3-benzothiazole-2-amine and with at least one synergistic agent selected from the group consisting of fluoxetine, zonisamide, and statins.
